(19) United States
(12) Patent Application Publication
(10) Pub. No.: US 2026/0055420 A1
(43) Pub. Date: Feb. 26, 2026

(54) CIS-ACTING REGULATORY ELEMENTS

(71) Applicant: PIONEER HI-BRED INTERNATIONAL, INC., JOHNSTON, IA (US)

(72) Inventors: MONIQUE COY, ANKENY, IA (US); SUNIL KUMAR, HYDERABAD (IN)

(73) Assignee: PIONEER HI-BRED INTERNATIONAL, INC., JOHNSTON, IA (US)

(21) Appl. No.: 19/333,912

(22) Filed: Sep. 19, 2025

Related U.S. Application Data
(62) Division of application No. 17/905,293.
(60) Provisional application No. 62/984,831, filed on Mar. 4, 2020.

(51) Int. Cl. C12N 15/82

(52) U.S. Cl. CPC C12N 15/8217

(57) ABSTRACT

Cis-acting regulatory elements were identified and engineered as a modulating element within a regulatory element (for example, a promoter, 5′UTR, intron or 3′UTR). Next, the chimeric cis-acting regulatory element and promoter were assayed to determine if the cis-acting regulatory elements could enhance the expression of a downstream coding sequence operably linked to the chimeric cis-acting regulatory element and promoter. Disclosed are novel cis-acting regulatory elements that were identified to enhance the expression of a downstream coding sequence.

### CROSS-REFERENCE TO RELATED APPLICATION

This application is a divisional application of U.S. patent application Ser. No. 17/905,293 (published as U.S. Patent Publication No. 2025-0179512; ), filed Aug. 30, 2022, which claims the benefit of the filing date of U.S. Provisional Patent Application Ser. No. 62/984,831, filed Mar. 4, 2020, the disclosures of which are hereby incorporated by reference in their entirety.

### REFERENCE TO A SEQUENCE LISTING

The official copy of the sequence listing is submitted electronically via EFS-Web as an XML formatted sequence listing with a file named “8424-WO-PCT-Sequence listing XML File.xml”, created on Nov. 6, 2025, and having a size of 1,950,751 bytes and is filed concurrently with the specification. The sequence listing contained in this XML formatted document is part of the specification and is herein incorporated by reference in its entirety.

### FIELD OF THE INVENTION

This disclosure is generally related to the field of molecular biology, and in particular embodiments to cis-acting regulatory elements that can be engineered in proximity to a coding sequence such that the expression of the coding sequence is increased. In certain aspects, the cis-acting elements are engineered with a regulatory element (for example a promoter, 5′UTR, intron or 3′UTR) to produce a novel chimeric sequence. In other aspects, the novel chimeric sequence comprised of a cis-acting regulatory element and another regulatory element (for example a promoter, 5′UTR, intron or 3′UTR) drive higher levels of expression of a coding sequence. In further aspects, the cis-acting regulatory element functions within a living cell (for example a plant cell) to drive higher levels of expression of a coding sequence. Accordingly, this disclosure provides compositions and methods for the identification, detection and utilization of such a cis-acting regulatory element.

### BACKGROUND

Many plant species are capable of being transformed with transgenes to introduce agronomically desirable traits or characteristics. The resulting plant species are developed and/or modified to have particular desirable traits. Generally, desirable traits include, for example, improving nutritional value quality, increasing yield, conferring pest or disease resistance, increasing drought and stress tolerance, improving horticultural qualities (e.g., pigmentation and growth), imparting herbicide tolerance, enabling the production of industrially useful compounds and/or materials from the plant, and/or enabling the production of pharmaceuticals.

Transgenic plant species comprising multiple transgenes stacked at a single genomic locus are produced via plant transformation technologies. Plant transformation technologies result in the introduction of a transgene into a plant cell, recovery of a fertile transgenic plant that contains the stably integrated copy of the transgene in the plant genome, and subsequent transgene expression via transcription and translation results in transgenic plants that possess desirable traits and phenotypes. However, novel gene regulatory elements that allow the production of transgenic plant species to highly express multiple transgenes engineered as a trait stack are desirable.

Likewise, novel gene regulatory elements that allow the expression of a transgene within particular tissues or organs of a plant are desirable. For example, increased resistance of a plant to infection by soil-borne pathogens might be accomplished by transforming the plant genome with a pathogen-resistance gene such that pathogen-resistance protein is robustly expressed within the roots of the plant. Alternatively, it may be desirable to express a transgene in plant tissues that are in a particular growth or developmental phase such as, for example, cell division or elongation. Furthermore, it may be desirable to express a transgene in leaf and stem tissues of a plant to provide tolerance against herbicides, or resistance against above ground insects and pests.

Therefore, a need exists for new gene regulatory elements that can drive the desired levels of expression of transgenes in specific plant tissues. Accordingly, there remains a need for compositions and methodologies to increase expression of a coding sequence such that the expression of the coding sequence is robustly driven by cis-acting regulating and other regulatory elements (for example a promoter).

### SUMMARY

Disclosed herein are sequences, constructs, and methods for a chimeric regulatory molecule wherein the molecule comprises a nucleic acid sequence comprising at least 90%, 91%, 92%, 93%, 94%, 95%, 96%, 97%, 98%, 99%, or 100% identity to SEQ ID NO:9, SEQ ID NOs:25-583, SEQ ID NOs:585-2268. In some aspects the chimeric regulatory molecule comprises SEQ ID NO:9, SEQ ID NOs:25-583, SEQ ID NOs:585-2268. In other aspects the chimeric regulatory molecule consists of SEQ ID NO:9, SEQ ID NOs:25-583, SEQ ID NOs:585-2268. In further aspects, the nucleic acid sequence comprising at least 90%, 91%, 92%, 93%, 94%, 95%, 96%, 97%, 98%, 99%, or 100% identity to SEQ ID NO:9, SEQ ID NOs:25-583, SEQ ID NOs:585-2268 functions as an enhancer. In additional aspects, the nucleic acid sequence comprising at least 90%, 91%, 92%, 93%, 94%, 95%, 96%, 97%, 98%, 99%, or 100% identity to SEQ ID NO:9, SEQ ID NOs:25-583, SEQ ID NOs:585-2268 functions as a repressor. In some aspects the chimeric regulatory molecule comprises a promoter operably linked to the nucleic acid sequence comprising at least 90%, 91%, 92%, 93%, 94%, 95%, 96%, 97%, 98%, 99%, or 100% identity to SEQ ID NO:9, SEQ ID NOs:25-583, SEQ ID NOs:585-2268. In further aspects the chimeric regulatory molecule comprises an intron operably linked to the nucleic acid sequence comprising at least 90%, 91%, 92%, 93%, 94%, 95%, 96%, 97%, 98%, 99%, or 100% identity to SEQ ID NO:9, SEQ ID NOs:25-583, SEQ ID NOs:585-2268. In other aspects the chimeric regulatory molecule comprises an 5′ UTR operably linked to the nucleic acid sequence comprising at least 90%, 91%, 92%, 93%, 94%, 95%, 96%, 97%, 98%, 99%, or 100% identity to SEQ ID NO:9, SEQ ID NOs:25-583, SEQ ID NOs:585-2268. In additional aspects the nucleic acid sequence comprising at least 90%, 91%, 92%, 93%, 94%, 95%, 96%, 97%, 98%, 99%, or 100% identity to SEQ ID NO:9, SEQ ID NOs:25-583, SEQ ID NOs:585-2268. is provided as two or more copies.

Further disclosed herein is a gene expression cassette comprising the chimeric regulatory molecule, wherein the molecule comprises a nucleic acid sequence comprising at least 90%, 91%, 92%, 93%, 94%, 95%, 96%, 97%, 98%, 99%, or 100% identity to SEQ ID NO:9, SEQ ID NOs:25-583, SEQ ID NOs:585-2268. In some aspects the nucleic acid sequence comprising at least 90%, 91%, 92%, 93%, 94%, 95%, 96%, 97%, 98%, 99%, or 100% identity to SEQ ID NO:9, SEQ ID NOs:25-583, SEQ ID NOs:585-2268 is operably linked to a promoter, intron, of 5′UTR. In other aspects the chimeric regulatory molecule is operably linked to a heterologous coding sequence. In further aspects the heterologous coding sequence is a gene of interest. Examples of the gene of interest includes a selectable marker protein, an insecticidal resistance protein, a herbicide tolerance protein, a nitrogen use efficiency protein, a water use efficiency protein, a small RNA molecule, a nutritional quality protein, or a DNA binding protein. In further aspects the gene expression cassette is engineered within a recombinant vector. Examples of a vector include a plasmid, a cosmid, a bacterial artificial chromosome, a virus, and a bacteriophage. In additional aspects the nucleic acid sequence comprising at least 90%, 91%, 92%, 93%, 94%, 95%, 96%, 97%, 98%, 99%, or 100% identity SEQ ID NO:9, SEQ ID NOs:25-583, SEQ ID NOs:585-2268 is provided as two or more copies.

Disclosed herein is a transgenic plant cell comprising a chimeric regulatory molecule wherein the molecule comprises a nucleic acid sequence comprising at least 90%, 91%, 92%, 93%, 94%, 95%, 96%, 97%, 98%, 99%, or 100% identity SEQ ID NO:9, SEQ ID NOs:25-583, SEQ ID NOs:585-2268. In some aspects the transgenic plant cell comprises a gene expression cassette that comprises a chimeric regulatory molecule wherein the molecule comprises a nucleic acid sequence comprising at least 90%, 91%, 92%, 93%, 94%, 95%, 96%, 97%, 98%, 99%, or 100% identity to SEQ ID NO:9, SEQ ID NOs:25-583, SEQ ID NOs:585-2268. In other aspects the transgenic plant cell is a monocotyledonous plant. In further aspects the transgenic plant cell is a dicotyledonous plant. In additional aspects the gene expression cassette is expressed constitutively. In other aspects the transgenic plant is stably transformed with the gene expression cassette. In some aspects the transgenic plant is transiently transformed with the gene expression cassette. In additional aspects a seed comprising the gene expression cassette is developed from the transgenic plant. In further aspects a progeny plant comprising the gene expression cassette is developed from the transgenic plant.

Disclosed herein is a method for inhibiting weed growth in a field of transgenic herbicide tolerant crop plants comprising planting the transgenic plants transformed with a gene expression cassette comprising a chimeric regulatory molecule wherein the molecule comprises a nucleic acid sequence comprising at least 90%, 91%, 92%, 93%, 94%, 95%, 96%, 97%, 98%, 99%, or 100% identity to SEQ ID NO:9, SEQ ID NOs:25-583, SEQ ID NOs:585-2268, active in a plant cell and operably linked to a polynucleotide molecule encoding an herbicide tolerance gene; and applying herbicide to the field at an application rate that inhibits the growth of weeds, wherein the growth and yield of the transgenic crop plant is not substantially affected by the herbicide application. Disclosed herein is a method for providing pest control in a field of transgenic crop plants, the method comprising the steps of planting the transgenic plants transformed with a gene expression cassette comprising a chimeric regulatory molecule wherein the molecule comprises a nucleic acid sequence comprising at least 90%, 91%, 92%, 93%, 94%, 95%, 96%, 97%, 98%, 99%, or 100% identity to SEQ ID NO:9, SEQ ID NOs:25-583, SEQ ID NOs:585-2268, active in a plant cell and operably linked to a gene conferring pest resistance. Disclosed herein is a method for providing disease control in a field of transgenic crop plants, the method comprising the steps of planting the transgenic plants transformed with a gene expression cassette comprising at least 90%, 91%, 92%, 93%, 94%, 95%, 96%, 97%, 98%, 99%, or 100% identity to SEQ ID NO:9, SEQ ID NOs:25-583, SEQ ID NOs:585-2268, active in a plant cell and operably linked to a gene conferring disease resistance. Disclosed herein, is a method of providing stress tolerance to plants in a field of transgenic crop plants, the method comprising the steps of planting the transgenic plants transformed with a gene expression cassette comprising at least 90%, 91%, 92%, 93%, 94%, 95%, 96%, 97%, 98%, 99%, or 100% identity to SEQ ID NO:9, SEQ ID NOs:25-583, SEQ ID NOs:585-2268, active in a plant cell and operably linked to a gene conferring stress tolerance. Disclosed herein is a method of providing yield enhancement to plants in a field of transgenic crop plants, the method comprising the steps of planting the transgenic plants transformed with a gene expression cassette comprising at least 90%, 91%, 92%, 93%, 94%, 95%, 96%, 97%, 98%, 99%, or 100% identity to SEQ ID NO:9, SEQ ID NOs:25-583, SEQ ID NOs:585-2268, active in a plant cell and operably linked to a gene conferring yield enhancement.

Disclosed herein is a method for transforming a host cell, the method comprising the steps of: providing a chimeric regulatory molecule comprising a polynucleotide that shares at least 90%, 91%, 92%, 93%, 94%, 95%, 96%, 97%, 98%, 99%, or 100% identity to SEQ ID NO:9, SEQ ID NOs:25-583, SEQ ID NOs:585-2268 wherein said molecule is operably linked to a heterologous coding sequence, and transforming said cell with the nucleic acid molecule. In some aspects, the plant transformation method is selected from an Agrobacterium-mediated transformation method, a biolistics transformation method, a silicon carbide transformation method, a protoplast transformation method, and a liposome transformation method. Disclosed herein is a method for producing a transgenic plant cell, the method comprising the steps of: transforming a plant cell with a gene expression cassette comprising a chimeric regulatory molecule comprising a polynucleotide that shares at least 90%, 91%, 92%, 93%, 94%, 95%, 96%, 97%, 98%, 99%, or 100% identity to SEQ ID NO:9, SEQ ID NOs:25-583, SEQ ID NOs:585-2268 operably linked to at least one polynucleotide sequence of interest; isolating the transformed plant cell comprising the gene expression cassette; and, producing a transgenic plant cell comprising the chimeric regulatory molecule a chimeric regulatory molecule comprising a polynucleotide that shares at least 90%, 91%, 92%, 93%, 94%, 95%, 96%, 97%, 98%, 99%, or 100% identity to SEQ ID NO:9, SEQ ID NOs:25-583, SEQ ID NOs:585-2268 operably linked to at least one polynucleotide sequence of interest. In some aspect the plant cell is transformed with a plant transformation method. In other aspects, the plant transformation method is selected from an Agrobacterium-mediated transformation method, a biolistics transformation method, a silicon carbide transformation method, a protoplast transformation method, and a liposome transformation method. In further aspect the polynucleotide sequence of interest is expressed in a plant cell. In additional aspects the polynucleotide sequence of interest is stably integrated into the genome of the transgenic plant cell. In other aspect the polynucleotide sequence of interest is transiently integrated into the genome of the transgenic plant cell. In further aspects the method further comprising the steps of: regenerating the transgenic plant cell into a transgenic plant; and, obtaining the transgenic plant, wherein the transgenic plant comprises the gene expression cassette comprising the chimeric regulatory molecule of claim 1 operably linked to at least one polynucleotide sequence of interest. In additional aspects the transgenic plant cell is a monocotyledonous transgenic plant cell or a dicotyledonous transgenic plant cell. In still other aspects the polynucleotide sequence of interest is a trait selected from the group consisting of an insecticidal resistance trait, herbicide tolerance trait, nitrogen use efficiency trait, water use efficiency trait, nutritional quality trait, DNA binding trait, selectable marker trait, small RNA trait, or any combination thereof. In other aspect the polynucleotide that shares at least 90%, 91%, 92%, 93%, 94%, 95%, 96%, 97%, 98%, 99%, or 100% identity to SEQ ID NO:9, SEQ ID NOs:25-583, SEQ ID NOs:585-2268 is provided in two or more copies.

Disclosed herein is a transgenic plant stably transformed with a polynucleotide molecule comprising: a chimeric regulatory molecule comprising a nucleic acid sequence selected from the group consisting of: a nucleic acid sequence comprising at least 90%, 91%, 92%, 93%, 94%, 95%, 96%, 97%, 98%, 99%, or 100% identity to SEQ ID NO:9, SEQ ID NOs:25-583, SEQ ID NOs:585-2268 and has promoter activity; a fragment comprising at least 4-33 contiguous nucleotides of SEQ ID NO:9, SEQ ID NOs:25-583, SEQ ID NOs:585-2268 and has promoter activity; and the nucleic acid sequence of SEQ ID NO:9, SEQ ID NOs:25-583, SEQ ID NOs:585-2268; a heterologous coding sequence, wherein the chimeric regulatory molecule is operably linked to the heterologous coding sequence. In some aspects the transgenic plant is transformed with a plant transformation method. Exemplary plant transformation methods include Agrobacterium-mediated transformation method, a biolistics transformation method, a silicon carbide transformation method, a protoplast transformation method, and a liposome transformation method. In other aspects the heterologous coding sequence is expressed in a cell of the transgenic plant. In further aspects the transgenic plant is a monocotyledonous transgenic plant cell or a dicotyledonous transgenic plant cell. Exemplary heterologous coding sequences include a trait selected from the group consisting of an insecticidal resistance trait, herbicide tolerance trait, nitrogen use efficiency trait, water use efficiency trait, nutritional quality trait, DNA binding trait, selectable marker trait, small RNA trait, or any combination thereof. In some aspects the transgenic plant produces a commodity product. Exemplary commodity products include protein concentrate, protein isolate, grain, meal, flour, oil, or fiber.

Disclosed herein is a method for enhancing the expression of a regulatory molecule, the method comprising the steps of: obtaining a cis-acting regulatory element comprising at least 90%, 91%, 92%, 93%, 94%, 95%, 96%, 97%, 98%, 99%, or 100% identity to SEQ ID NO:9, SEQ ID NOs:25-583, SEQ ID NOs:585-2268; engineering the cis-acting regulatory element comprising at least 90%, 91%, 92%, 93%, 94%, 95%, 96%, 97%, 98%, 99%, or 100% identity to SEQ ID NO:9, SEQ ID NOs:25-583, SEQ ID NOs:585-2268 within a regulatory molecule to produce a chimeric regulatory molecule; linking the chimeric regulatory molecule to a heterologous coding sequence to produce a gene expression cassette; transforming the gene expression cassette within a plant to obtain a transgenic plant, wherein the transgenic plant expresses the heterologous coding sequence. In some aspects the regulatory molecule is a promoter. In other aspects transformation method is selected from the group consisting of an Agrobacterium-mediated transformation method, a biolistics transformation method, a silicon carbide transformation method, a protoplast transformation method, and a liposome transformation method. In further aspects the regulatory molecule is expressed within a cell of the transgenic plant. In additional aspects the polynucleotide sequence of interest is stably integrated into the genome of the plant cell. In subsequent aspects the transgenic plant is a monocotyledonous plant or a dicotyledonous plant. In additional aspects the cis-acting regulatory element is provided as two or more copies.

Disclosed herein is an isolated nucleic acid molecule capable of modulating transcription of a gene of interest, wherein the nucleic acid molecule comprises at least 90%, 91%, 92%, 93%, 94%, 95%, 96%, 97%, 98%, 99%, or 100% identity to SEQ ID NO:9, SEQ ID NOs:25-583, SEQ ID NOs:585-2268. In some aspects the modulation results in enhanced expression of the gene of interest. In other aspects the modulation results in reduced expression of the gene of interest. In additional aspects the modulation of expression of the gene of interest occurs in a eukaryotic cell. In other aspects the gene of interest is a trait selected from the group consisting of an insecticidal resistance trait, herbicide tolerance trait, nitrogen use efficiency trait, water use efficiency trait, nutritional quality trait, DNA binding trait, selectable marker trait, small RNA trait, or any combination thereof. Disclosed herein is a chimeric regulatory molecule produced by a method comprising the steps of: introducing at least one cis-acting regulatory element within a promoter regulatory element to produce a chimeric regulatory molecule, wherein the cis-acting regulatory element comprises a nucleic acid molecule that shares at least 90%, 91%, 92%, 93%, 94%, 95%, 96%, 97%, 98%, 99%, or 100% identity to SEQ ID NO:9, SEQ ID NOs:25-583, SEQ ID NOs:585-2268. In other aspects the chimeric regulatory molecule comprises a heterologous coding sequence operably linked to the chimeric regulatory molecule. In other aspects the heterologous coding sequence is a trait selected from the group consisting of an insecticidal resistance trait, herbicide tolerance trait, nitrogen use efficiency trait, water use efficiency trait, nutritional quality trait, DNA binding trait, selectable marker trait, small RNA trait, or any combination thereof. In further aspects the chimeric regulatory molecule is introduced into a gene expression cassette. In other aspects the method includes the steps of: transforming a plant cell with the gene expression cassette; and, obtaining a transgenic plant cell comprising the gene expression cassette. In additional aspect the method includes the steps of: screening the transgenic plant for expression of the heterologous coding sequence; detecting the levels of the expressed heterologous coding sequence to determine an expression profile of the chimeric regulatory molecule; and, comparing the expression profile of the chimeric regulatory molecule to an expression profile of a transgenic plant that expresses a heterologous coding sequence driven by the promoter regulatory element, wherein the promoter regulatory element does not contain the cis-acting element. In additional aspects the nucleic acid sequence comprising at least 90%, 91%, 92%, 93%, 94%, 95%, 96%, 97%, 98%, 99%, or 100% identity to SEQ ID NO:9, SEQ ID NOs:25-583, SEQ ID NOs:585-2268 is provided as two or more copies.

The foregoing and other features will become more apparent from the following embodiments as provided in the Claims and Detailed Description, which proceeds with reference to the accompanying Figures and Sequence Listing.

### FIGURES

FIG. 1 provides a graph showing the effect of a select set of cis-acting regulatory elements obtained from a database on expression levels driven by the CaMV 35S minimum promoter. EE1906 is background control, i.e., CaMV 35S minimum promoter with no test cassette. Error bars are shown as SEM.

FIG. 2 provides a table showing a sequence alignment of SEQ ID NO:23-32.

FIG. 3 provides a table showing a sequence alignment of SEQ ID NO:33-36.

FIG. 4 provides a graph showing the effect of MMV as-1 elements on expression levels driven by the CaMV 35S minimum promoter. EE1906 is background control, i.e., CaMV 35S minimum promoter with no test cassette. Error bars are shown as SEM.

FIG. 5 provides a graph showing the effect of FMV as-1 elements on expression levels driven by the CaMV 35S minimum promoter. EE1906 is background control, i.e., CaMV 35S minimum promoter with no test cassette. Error bars are shown as SEM.

FIG. 6 provides a graph showing the effect of MMV as-1 elements on expression levels driven by the CaMV 35S minimum promoter. EE1906 is background control, i.e., CaMV 35S minimum promoter with no test cassette. Error bars are shown as SEM.

FIG. 7 and FIG. 7.1 provides a table showing a sequence alignment of SEQ ID NO:508-515.

FIG. 8 provides a graph showing the effect of MMV as-1 elements on expression levels driven by the CaMV 35S minimum promoter. EE1906 is background control, i.e., CaMV 35S minimum promoter with no test cassette. Error bars are shown as SEM.

FIG. 9 provides a table showing a sequence alignment of SEQ ID NO:516-525.

FIG. 10 provides a graph showing the effect of PSGS3AF1 elements on expression levels driven by the CaMV 35S minimum promoter. EE1906 is background control, i.e., CaMV 35S minimum promoter with no test cassette. Error bars are shown as SEM.

FIG. 11 provides a table showing a sequence alignment of SEQ ID NO:526-531.

FIG. 12 provides a graph showing the effect of cis-genic regulatory elements on expression levels driven by the Zea mays GOS2 minimum promoter. EE1619 is background control, i.e., CaMV 35S minimum promoter with no test cassette. Error bars are shown as SEM.

FIG. 13 and FIG. 13.1 provides a table showing a sequence alignment of SEQ ID NO:532-550 and SEQ ID NO:25, 28, 30 and 33.

FIG. 14 provides a graph showing the effect of cis-genic regulatory elements on expression levels driven by the Zea mays GOS2 promoter. EE1619 is background control, i.e., CaMV 35S minimum promoter with no test cassette. Error bars are shown as SEM.

FIG. 15, FIG. 15.1, and FIG. 15.2 provides a table showing a sequence alignment of SEQ ID NO:551-583 and SEQ ID NO:2, 3, 4, 5, 8, 9.

FIG. 16 provides a graph showing the effect of MMV-EME 1 and GM-PSGS3AF1-V3 elements on expression levels driven by the Zea mays GOS2 promoter. EE1619 is the background control, i.e., CaMV 35S minimum promoter with no test cassette. Error bars are shown as SEM.

FIG. 17 provides a table showing a sequence alignment of SEQ ID NO:516 and 529.

FIG. 18 provides a graph showing the effect of MMV as-1 elements obtained from maize on expression levels driven by the CaMV 35S minimum promoter. EE3549 is the background control, i.e., CaMV 35S minimum promoter (Yang et al., 2000) with no test cassette. Error bars are shown as SEM.

FIG. 19 and FIG. 19.1 provides a table showing a sequence alignment of SEQ ID NO:585-601.

FIG. 20 provides a graph showing the effect of GVBAVAS1 elements on expression levels driven by the CaMV 35S minimum promoter. EE3549 is a background control, i.e., CaMV 35S minimum promoter (Yang et al., 2000) with no test cassette. Error bars are shown as SEM.

FIG. 21, FIG. 21.1 and FIG. 21.2 provides a table showing a sequence alignment of SEQ ID NO:602-634.

### SEQUENCE LISTING

The nucleic acid sequences listed in the accompanying sequence listing are shown using standard letter abbreviations for nucleotide bases, as defined in 37 C.F.R. § 1.822. Only one strand of each nucleic acid sequence is shown, but the complementary strand and reverse complementary strand are understood as included by any reference to the displayed strand. As the complement and reverse complement of a primary nucleic acid sequence are necessarily disclosed by the primary sequence, the complementary sequence and reverse complementary sequence of a nucleic acid sequence are included by any reference to the nucleic acid sequence, unless it is explicitly stated to be otherwise (or it is clear to be otherwise from the context in which the sequence appears).

### DETAILED DESCRIPTION

Development of transgenic plant products is becoming increasingly complex. Commercially viable transgenic plants now require the stacking of multiple transgenes into a single locus. Obtaining optimal levels of transgene/heterologous coding sequence expression is necessary for the production of a single polygenic trait. Unfortunately, gene expression constructs driven by a weakly expressing promoter may not be expressed at an optimal expression level, thereby resulting in less efficacious transgenic products in the field. Accordingly there remains a need to increase expression of a transgene/heterologous coding sequence within a plant to develop transgenic crops that robustly express the transgene/heterologous coding sequence.

Provided are methods and compositions for overcoming such problems through the use of cis-acting regulatory elements to express transgenes in planta.

### Definitions

Unless defined otherwise, all technical and scientific terms used herein have the same meaning as commonly understood by one of ordinary skill in the art to which this disclosure relates. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference, unless only specific sections of patents or patent publications are indicated to be incorporated by reference.

In order to further clarify this disclosure, the following terms, abbreviations and definitions are provided.

As used herein, the terms “comprises”, “comprising”, “includes”, “including”, “has”, “having”, “contains”,” or “containing”, or any other variation thereof, are intended to be non-exclusive or open-ended. For example, a composition, a mixture, a process, a method, an article, or an apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of an embodiment of the disclosure are intended to be nonrestrictive regarding the number of instances, i.e., occurrences of the element or component. Therefore “a” or “an” should be read to include one or at least one, and the singular word form of the element or component also includes the plural unless the number is obviously meant to be singular.

The term “invention” or “present invention” as used herein is a non-limiting term and is not intended to refer to any single embodiment of the particular invention but encompasses all possible embodiments as disclosed in the application.

“The term “isolated”, as used herein means having been removed from its natural environment, or removed from other compounds present when the compound is first formed. The term “isolated” embraces materials isolated from natural sources as well as materials (e.g., nucleic acids and proteins) recovered after preparation by recombinant expression in a host cell, or chemically-synthesized compounds such as nucleic acid molecules, proteins, and peptides.

The term “purified”, as used herein relates to the isolation of a molecule or compound in a form that is substantially free of contaminants normally associated with the molecule or compound in a native or natural environment, or substantially enriched in concentration relative to other compounds present when the compound is first formed, and means having been increased in purity as a result of being separated from other components of the original composition. The term “purified nucleic acid” is used herein to describe a nucleic acid sequence which has been separated, produced apart from, or purified away from other biological compounds including, but not limited to polypeptides, lipids and carbohydrates, while effecting a chemical or functional change in the component (e.g., a nucleic acid may be purified from a chromosome by removing protein contaminants and breaking chemical bonds connecting the nucleic acid to the remaining DNA in the chromosome).

The term “synthetic”, as used herein refers to a polynucleotide (i.e., a DNA or RNA) molecule that was created via chemical synthesis as an in vitro process. For example, a synthetic DNA may be created during a reaction within an Eppendorf™ tube, such that the synthetic DNA is enzymatically produced from a native strand of DNA or RNA. Other laboratory methods may be utilized to synthesize a polynucleotide sequence. Oligonucleotides may be chemically synthesized on an oligo synthesizer via solid-phase synthesis using phosphoramidites. The synthesized oligonucleotides may be annealed to one another as a complex, thereby producing a “synthetic” polynucleotide. Other methods for chemically synthesizing a polynucleotide are known in the art, and can be readily implemented for use in the present disclosure.

The term “about” as used herein means greater or lesser than the value or range of values stated by 10 percent, but is not intended to designate any value or range of values to only this broader definition. Each value or range of values preceded by the term “about” is also intended to encompass the embodiment of the stated absolute value or range of values.

For the purposes of the present disclosure, a “gene,” includes a DNA region encoding a gene product (see infra), as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, introns and locus control regions.

As used herein the terms “native” or “natural” define a condition found in nature. A “native DNA sequence” is a DNA sequence present in nature that was produced by natural means or traditional breeding techniques but not generated by genetic engineering (e.g., using molecular biology/transformation techniques).

As used herein a “transgene” is defined to be a nucleic acid sequence that encodes a gene product, including for example, but not limited to, an mRNA. In one embodiment the transgene/heterologous coding sequence is an exogenous nucleic acid, where the transgene/heterologous coding sequence has been introduced into a host cell by genetic engineering (or the progeny thereof) where the transgene/heterologous coding sequence is not normally found. In one example, a transgene/heterologous coding sequence encodes an industrially or pharmaceutically useful compound, or a gene encoding a desirable agricultural trait (e.g., an herbicide-resistance gene). In yet another example, a transgene/heterologous coding sequence is an antisense nucleic acid sequence, wherein expression of the antisense nucleic acid sequence inhibits expression of a target nucleic acid sequence. In one embodiment the transgene/heterologous coding sequence is an endogenous nucleic acid, wherein additional genomic copies of the endogenous nucleic acid are desired, or a nucleic acid that is in the antisense orientation with respect to the sequence of a target nucleic acid in a host organism.

As used herein the term “non-GmPSID2 transgene” or “non-GmPSID2 gene” is any transgene/heterologous coding sequence that has less than 80% sequence identity with the GmPSID2 gene coding sequence.

As used herein, “heterologous DNA coding sequence” means any coding sequence other than the one that naturally encodes the GmPSID2 gene, or any homolog of the expressed GmPSID2 protein. The term “heterologous” is used in the context of this invention for any combination of nucleic acid sequences that is not normally found intimately associated in nature.

A “gene product” as defined herein is any product produced by the gene. For example the gene product can be the direct transcriptional product of a gene (e.g., mRNA, tRNA, IRNA, antisense RNA, interfering RNA, ribozyme, structural RNA or any other type of RNA) or a protein produced by translation of a mRNA. Gene products also include RNAs which are modified, by processes such as capping, polyadenylation, methylation, and editing, and proteins modified by, for example, methylation, acetylation, phosphorylation, ubiquitination, ADP-ribosylation, myristoylation, and glycosylation. Gene expression can be influenced by external signals, for example, exposure of a cell, tissue, or organism to an agent that increases or decreases gene expression. Expression of a gene can also be regulated anywhere in the pathway from DNA to RNA to protein. Regulation of gene expression occurs, for example, through controls acting on transcription, translation, RNA transport and processing, degradation of intermediary molecules such as mRNA, or through activation, inactivation, compartmentalization, or degradation of specific protein molecules after they have been made, or by combinations thereof. Gene expression can be measured at the RNA level or the protein level by any method known in the art, including, without limitation, Northern blot, RT-PCR, Western blot, or in vitro, in situ, or in vivo protein activity assay(s).

As used herein the term “gene expression” relates to the process by which the coded information of a nucleic acid transcriptional unit (including, e.g., genomic DNA) is converted into an operational, non-operational, or structural part of a cell, often including the synthesis of a protein. Gene expression can be influenced by external signals; for example, exposure of a cell, tissue, or organism to an agent that increases or decreases gene expression. Expression of a gene can also be regulated anywhere in the pathway from DNA to RNA to protein. Regulation of gene expression occurs, for example, through controls acting on transcription, translation, RNA transport and processing, degradation of intermediary molecules such as mRNA, or through activation, inactivation, compartmentalization, or degradation of specific protein molecules after they have been made, or by combinations thereof. Gene expression can be measured at the RNA level or the protein level by any method known in the art, including, without limitation, Northern blot, RT-PCR, Western blot, or in vitro, in situ, or in vivo protein activity assay(s).

As used herein, “homology-based gene silencing” (HBGS) is a generic term that includes both transcriptional gene silencing and post-transcriptional gene silencing. Silencing of a target locus by an unlinked silencing locus can result from transcription inhibition (transcriptional gene silencing; TGS) or mRNA degradation (post-transcriptional gene silencing; PTGS), owing to the production of double-stranded RNA (dsRNA) corresponding to promoter or transcribed sequences, respectively. The involvement of distinct cellular components in each process suggests that dsRNA-induced TGS and PTGS likely result from the diversification of an ancient common mechanism. However, a strict comparison of TGS and PTGS has been difficult to achieve because it generally relies on the analysis of distinct silencing loci. In some instances, a single transgene locus can triggers both TGS and PTGS, owing to the production of dsRNA corresponding to promoter and transcribed sequences of different target genes. Mourrain et al. (2007) Planta 225:365-79. It is likely that siRNAs are the actual molecules that trigger TGS and PTGS on homologous sequences: the siRNAs would in this model trigger silencing and methylation of homologous sequences in cis and in trans through the spreading of methylation of transgene sequences into the endogenous promoter.

As used herein, the term “nucleic acid molecule” (or “nucleic acid” or “polynucleotide”) may refer to a polymeric form of nucleotides, which may include both sense and anti-sense strands of RNA, cDNA, genomic DNA, and synthetic forms and mixed polymers of the above. A nucleotide may refer to a ribonucleotide, deoxyribonucleotide, or a modified form of either type of nucleotide. A “nucleic acid molecule” as used herein is synonymous with “nucleic acid” and “polynucleotide”. A nucleic acid molecule is usually at least 10 bases in length, unless otherwise specified. The term may refer to a molecule of RNA or DNA of indeterminate length. The term includes single- and double-stranded forms of DNA. A nucleic acid molecule may include either or both naturally-occurring and modified nucleotides linked together by naturally occurring and/or non-naturally occurring nucleotide linkages.

Nucleic acid molecules may be modified chemically or biochemically, or may contain non-natural or derivatized nucleotide bases, as will be readily appreciated by those of skill in the art. Such modifications include, for example, labels, methylation, substitution of one or more of the naturally occurring nucleotides with an analog, internucleotide modifications (e.g., uncharged linkages: for example, methyl phosphonates, phosphotriesters, phosphoramidites, carbamates, etc.; charged linkages: for example, phosphorothioates, phosphorodithioates, etc.; pendent moieties: for example, peptides; intercalators: for example, acridine, psoralen, etc.; chelators; alkylators; and modified linkages: for example, alpha anomeric nucleic acids, etc.). The term “nucleic acid molecule” also includes any topological conformation, including single-stranded, double-stranded, partially duplexed, triplexed, hairpinned, circular, and padlocked conformations.

Transcription proceeds in a 5′ to 3′ manner along a DNA strand. This means that RNA is made by the sequential addition of ribonucleotide-5′-triphosphates to the 3′ terminus of the growing chain (with a requisite elimination of the pyrophosphate). In either a linear or circular nucleic acid molecule, discrete elements (e.g., particular nucleotide sequences) may be referred to as being “upstream” or “5′” relative to a further element if they are bonded or would be bonded to the same nucleic acid in the 5′ direction from that element. Similarly, discrete elements may be “downstream” or “3” relative to a further element if they are or would be bonded to the same nucleic acid in the 3′ direction from that element.

A base “position”, as used herein, refers to the location of a given base or nucleotide residue within a designated nucleic acid. The designated nucleic acid may be defined by alignment (see below) with a reference nucleic acid.

Hybridization relates to the binding of two polynucleotide strands via Hydrogen bonds. Oligonucleotides and their analogs hybridize by hydrogen bonding, which includes Watson-Crick, Hoogsteen or reversed Hoogsteen hydrogen bonding, between complementary bases. Generally, nucleic acid molecules consist of nitrogenous bases that are either pyrimidines (cytosine (C), uracil (U), and thymine (T)) or purines (adenine (A) and guanine (G)). These nitrogenous bases form hydrogen bonds between a pyrimidine and a purine, and the bonding of the pyrimidine to the purine is referred to as “base pairing.” More specifically, A will hydrogen bond to Tor U, and G will bond to C. “Complementary” refers to the base pairing that occurs between two distinct nucleic acid sequences or two distinct regions of the same nucleic acid sequence.

“Specifically hybridizable” and “specifically complementary” are terms that indicate a sufficient degree of complementarity such that stable and specific binding occurs between the oligonucleotide and the DNA or RNA target. The oligonucleotide need not be 100% complementary to its target sequence to be specifically hybridizable. An oligonucleotide is specifically hybridizable when binding of the oligonucleotide to the target DNA or RNA molecule interferes with the normal function of the target DNA or RNA, and there is sufficient degree of complementarity to avoid non-specific binding of the oligonucleotide to non-target sequences under conditions where specific binding is desired, for example under physiological conditions in the case of in vivo assays or systems. Such binding is referred to as specific hybridization.

Hybridization conditions resulting in particular degrees of stringency will vary depending upon the nature of the chosen hybridization method and the composition and length of the hybridizing nucleic acid sequences. Generally, the temperature of hybridization and the ionic strength (especially the Na+ and/or Mg2+ concentration) of the hybridization buffer will contribute to the stringency of hybridization, though wash times also influence stringency. Calculations regarding hybridization conditions required for attaining particular degrees of stringency are discussed in Sambrook et al. (ed.), μMolecular Cloning: A Laboratory Manual, 2nd ed., vol. 1-3, Cold Spring Harbor Laboratory Press, Cold Spring Harbor, New York, 1989, chs. 9 and 11.

As used herein, “stringent conditions” encompass conditions under which hybridization will only occur if there is less than 50% mismatch between the hybridization molecule and the DNA target. “Stringent conditions” include further particular levels of stringency. Thus, as used herein, “moderate stringency” conditions are those under which molecules with more than 50% sequence mismatch will not hybridize; conditions of “high stringency” are those under which sequences with more than 20% mismatch will not hybridize; and conditions of “very high stringency” are those under which sequences with more than 10% mismatch will not hybridize.

In particular embodiments, stringent conditions can include hybridization at 65° C., followed by washes at 65° C. with 0.1×SSC/0.1% SDS for 40 minutes.

The following are representative, non-limiting hybridization conditions:
Very High Stringency: Hybridization in 5×SSC buffer at 65° C. for 16 hours; wash twice in 2×SSC buffer at room temperature for 15 minutes each; and wash twice in 0.5×SSC buffer at 65° C. for 20 minutes each.
High Stringency: Hybridization in 5×-6×SSC buffer at 65-70° C. for 16-20 hours; wash twice in 2×SSC buffer at room temperature for 5-20 minutes each; and wash twice in 1×SSC buffer at 55-70° C. for 30 minutes each.
Moderate Stringency: Hybridization in 6×SSC buffer at room temperature to 55° C. for 16-20 hours; wash at least twice in 2×-3×SSC buffer at room temperature to 55° C. for 20-30 minutes each.

In particular embodiments, specifically hybridizable nucleic acid molecules can remain bound under very high stringency hybridization conditions. In these and further embodiments, specifically hybridizable nucleic acid molecules can remain bound under high stringency hybridization conditions. In these and further embodiments, specifically hybridizable nucleic acid molecules can remain bound under moderate stringency hybridization conditions.

As used herein, the term “oligonucleotide” refers to a short nucleic acid polymer. Oligonucleotides may be formed by cleavage of longer nucleic acid segments, or by polymerizing individual nucleotide precursors. Automated synthesizers allow the synthesis of oligonucleotides up to several hundred base pairs in length. Because oligonucleotides may bind to a complementary nucleotide sequence, they may be used as probes for detecting DNA or RNA. Oligonucleotides composed of DNA (oligodeoxyribonucleotides) may be used in PCR, a technique for the amplification of small DNA sequences. In PCR, the oligonucleotide is typically referred to as a “primer”, which allows a DNA polymerase to extend the oligonucleotide and replicate the complementary strand.

The terms “percent sequence identity” or “percent identity” or “identity” are used interchangeably to refer to a sequence comparison based on identical matches between correspondingly identical positions in the sequences being compared between two or more amino acid or nucleotide sequences. The percent identity refers to the extent to which two optimally aligned polynucleotide or peptide sequences are invariant throughout a window of alignment of components, e.g., nucleotides or amino acids. Hybridization experiments and mathematical algorithms known in the art may be used to determine percent identity. Many mathematical algorithms exist as sequence alignment computer programs known in the art that calculate percent identity. These programs may be categorized as either global sequence alignment programs or local sequence alignment programs.

Global sequence alignment programs calculate the percent identity of two sequences by comparing alignments end-to-end in order to find exact matches, dividing the number of exact matches by the length of the shorter sequences, and then multiplying by 100. Basically, the percentage of identical nucleotides in a linear polynucleotide sequence of a reference (“query) polynucleotide molecule as compared to a test (“subject”) polynucleotide molecule when the two sequences are optimally aligned (with appropriate nucleotide insertions, deletions, or gaps).

Local sequence alignment programs are similar in their calculation, but only compare aligned fragments of the sequences rather than utilizing an end-to-end analysis. Local sequence alignment programs such as BLAST can be used to compare specific regions of two sequences. A BLAST comparison of two sequences results in an E-value, or expectation value, that represents the number of different alignments with scores equivalent to or better than the raw alignment score, S, that are expected to occur in a database search by chance. The lower the E value, the more significant the match. Because database size is an element in E-value calculations, E-values obtained by BLASTing against public databases, such as GENBANK, have generally increased over time for any given query/entry match. In setting criteria for confidence of polypeptide function prediction, a “high” BLAST match is considered herein as having an E-value for the top BLAST hit of less than 1E-30; a medium BLASTX E-value is 1E-30 to 1E-8; and a low BLASTX E-value is greater than 1E-8. The protein function assignment in the present invention is determined using combinations of E-values, percent identity, query coverage and hit coverage. Query coverage refers to the percent of the query sequence that is represented in the BLAST alignment. Hit coverage refers to the percent of the database entry that is represented in the BLAST alignment. In one embodiment of the invention, function of a query polypeptide is inferred from function of a protein homolog where either (1) hit_p<1e-30 or % identity>35% AND query_coverage>50% AND hit_coverage>50%, or (2) hit_p<1e-8 AND query_coverage>70% AND hit_coverage>70%. The following abbreviations are produced during a BLAST analysis of a sequence.

| | |
|---|---|
| SEQ_NUM | provides the SEQ ID NO for the listed recombinant |
| | polynucleotide sequences. |
| CONTIG_ID | provides an arbitrary sequence name taken from the name of |
| | the clone from which the cDNA sequence was obtained. |
| PROTEIN_NUM | provides the SEQ ID NO for the recombinant polypeptide |
| | sequence |
| NCBI_GI | provides the GenBank ID number for the top BLAST hit for |
| | the sequence. The top BLAST hit is indicated by the |
| | National Center for Biotechnology Information GenBank |
| | Identifier number. |
| NCBI_GI_DESCRIPTION | refers to the description of the GenBank top BLAST hit for |
| | the sequence. |
| E_VALUE | provides the expectation value for the top BLAST match. |
| MATCH_LENGTH | provides the length of the sequence which is aligned in the |
| | top BLAST match |
| TOP_HIT_PCT_IDENT | refers to the percentage of identically matched nucleotides |
| | (or residues) that exist along the length of that portion of |
| | the sequences which is aligned in the top BLAST match. |
| CAT_TYPE | indicates the classification scheme used to classify the |
| | sequence. GO_BP = Gene Ontology Consortium -biological |
| | process; GO_CC = Gene Ontology Consortium - cellular |
| | component; GO_MF = Gene Ontology Consortium - molecular |
| | function; KEGG = KEGG functional hierarchy (KEGG = Kyoto |
| | Encyclopedia of Genes and Genomes); EC = Enzyme |
| | Classification from ENZYME data bank release 25.0; POI = |
| | Pathways of Interest. |
| CAT_DESC | provides the classification scheme subcategory to which the |
| | query sequence was assigned. |
| PRODUCT_CAT_DESC | provides the FunCAT annotation category to which the |
| | query sequence was assigned. |
| PRODUCT_HIT_DESC | provides the description of the BLAST hit which resulted in |
| | assignment of the sequence to the function category |
| | provided in the cat desc column. |
| HIT_E | provides the E value for the BLAST bit in the hit desc |
| | column. |
| PCT_IDENT | refers to the percentage of identically matched nucleotides |
| | (or residues) that exist along the length of that portion |
| | of the sequences which is aligned in the BLAST match |
| | provided in hit_desc. |
| QRY_RANGE | lists the range of the query sequence aligned with the hit. |
| HIT_RANGE | lists the range of the hit sequence aligned with the query. |
| QRY_CVRG | provides the percent of query sequence length that matches |
| | to the bit (NCBI) sequence in the BLAST match (% qry cvrg = |
| | (match length/query total length) × 100). |
| HIT_CVRG | provides the percent of hit sequence length that matches to |
| | the query sequence in the match generated using BLAST (% |
| | hit cvrg = (match length/hit total length) × 100) |

Methods for aligning sequences for comparison are well-known in the art. Various programs and alignment algorithms are described. In an embodiment, the subject disclosure relates to calculating percent identity between two polynucleotides or amino acid sequences using an AlignX alignment program of the Vector NTI suite (Invitrogen, Carlsbad, CA). The AlignX alignment program is a global sequence alignment program for polynucleotides or proteins. In an embodiment, the subject disclosure relates to calculating percent identity between two polynucleotides or amino acid sequences using the MegAlign program of the LASERGENE bioinformatics computing suite (MegAlign™ (©1993-2016). DNASTAR. Madison, WI). The MegAlign program is global sequence alignment program for polynucleotides or proteins. In an embodiment, the subject disclosure relates to calculating percent identity between two polynucleotides or amino acid sequences using the Clustal suite of alignment programs, including, but not limited to, ClustalW and ClustalV (Higgins and Sharp (1988) Gene. December 15; 73(1):237-44; Higgins and Sharp (1989) CABIOS 5:151-3; Higgins et al. (1992) Comput. Appl. Biosci. 8:189-91). In an embodiment, the subject disclosure relates to calculating percent identity between two polynucleotides or amino acid sequences using the GCG suite of programs (Wisconsin Package Version 9.0, Genetics Computer Group (GCG), Madison, WI). In an embodiment, the subject disclosure relates to calculating percent identity between two polynucleotides or amino acid sequences using the BLAST suite of alignment programs, for example, but not limited to, BLASTP, BLASTN, BLASTX, etc. (Altschul et al. (1990) J. Mol. Biol. 215:403-10). In an embodiment, the subject disclosure relates to calculating percent identity between two polynucleotides or amino acid sequences using the FASTA suite of alignment programs, including, but not limited to, FASTA, TFASTX, TFASTY, SSEARCH, LALIGN etc. (Pearson (1994) Comput. Methods Genome Res. [Proc. Int. Symp.], Meeting Date 1992 (Suhai and Sandor, Eds.), Plenum: New York, NY, pp. 111-20). In an embodiment, the subject disclosure relates to calculating percent identity between two polynucleotides or amino acid sequences using the T-Coffee alignment program (Notredame, et. al. (2000) J. Mol. Biol. 302, 205-17). In an embodiment, the subject disclosure relates to calculating percent identity between two polynucleotides or amino acid sequences using the DIALIGN suite of alignment programs, including, but not limited to DIALIGN, CHAOS, DIALIGN-TX, DIALIGN-T etc. (A1Ait, et. al. (2013) DIALIGN at GOBICS Nuc. Acids Research 41, W3-W7). In an embodiment, the subject disclosure relates to calculating percent identity between two polynucleotides or amino acid sequences using the MUSCLE suite of alignment programs (Edgar (2004) Nucleic Acids Res. 32(5):1792-1797). In an embodiment, the subject disclosure relates to calculating percent identity between two polynucleotides or amino acid sequences using the MAFFT alignment program (Katoh, et. al. (2002) Nucleic Acids Research 30(14):3059-3066). In an embodiment, the subject disclosure relates to calculating percent identity between two polynucleotides or amino acid sequences using the Genoogle program (Albrecht, Felipe. arXiv130702987v1 [cs.DC] 10 Jul. 2015). In an embodiment, the subject disclosure relates to calculating percent identity between two polynucleotides or amino acid sequences using the HMMER suite of programs (Eddy. (1998) Bioinformatics, 14:755-63). In an embodiment, the subject disclosure relates to calculating percent identity between two polynucleotides or amino acid sequences using the PLAST suite of alignment programs, including, but not limited to, TPLASTN, PLASTP, KLAST, and PLASTX (Nguyen & Lavenier. (2009) BMC Bioinformatics, 10:329). In an embodiment, the subject disclosure relates to calculating percent identity between two polynucleotides or amino acid sequences using the USEARCH alignment program (Edgar (2010) Bioinformatics 26(19), 2460-61). In an embodiment, the subject disclosure relates to calculating percent identity between two polynucleotides or amino acid sequences using the SAM suite of alignment programs (Hughey & Krogh (January 1995) Technical Report UCSC0CRL-95-7, University of California, Santa Cruz). In an embodiment, the subject disclosure relates to calculating percent identity between two polynucleotides or amino acid sequences using the IDF Searcher (O'Kane, K. C., The Effect of Inverse Document Frequency Weights on Indexed Sequence Retrieval, Online Journal of Bioinformatics, Volume 6 (2) 162-173, 2005). In an embodiment, the subject disclosure relates to calculating percent identity between two polynucleotides or amino acid sequences using the Parasail alignment program. (Daily, Jeff. Parasail: SIMD C library for global, semi-global, and local pairwise sequence alignments. BMC Bioinformatics. 17:18. Feb. 10, 2016). In an embodiment, the subject disclosure relates to calculating percent identity between two polynucleotides or amino acid sequences using the ScalaBLAST alignment program (Oehmen C, Nieplocha J. “ScalaBLAST: A scalable implementation of BLAST for high-performance data-intensive bioinformatics analysis.” IEEE Transactions on Parallel & Distributed Systems 17 (8): 740-749 August 2006). In an embodiment, the subject disclosure relates to calculating percent identity between two polynucleotides or amino acid sequences using the SWIPE alignment program (Rognes, T. Faster Smilth-Waterman database searches with inter-sequence SIMD parallelization. BMC Bioinformatics. 12, 221 (2011)). In an embodiment, the subject disclosure relates to calculating percent identity between two polynucleotides or amino acid sequences using the ACANA alignment program (Weichun Huang, David M. Umbach, and Leping Li, Accurate anchoring alignment of divergent sequences. Bioinformatics 22:29-34, Jan. 1 2006). In an embodiment, the subject disclosure relates to calculating percent identity between two polynucleotides or amino acid sequences using the DOTLET alignment program (Junier, T. & Pagni, M. DOTLET: diagonal plots in a web browser. Bioinformatics 16 (2): 178-9 February 2000). In an embodiment, the subject disclosure relates to calculating percent identity between two polynucleotides or amino acid sequences using the G-PAS alignment program (Frohmberg, W., et al. G-PAS 2.0—an improved version of protein alignment tool with an efficient backtracking routine on multiple GPUs. Bulletin of the Polish Academy of Sciences Technical Sciences, Vol. 60, 491 November 2012). In an embodiment, the subject disclosure relates to calculating percent identity between two polynucleotides or amino acid sequences using the GapMis alignment program (Flouri, T. et. al., Gap Mis: A tool for pairwise sequence alignment with a single gap. Recent Pat DNA Gene Seq. 7(2):84-95 August 2013). In an embodiment, the subject disclosure relates to calculating percent identity between two polynucleotides or amino acid sequences using the EMBOSS suite of alignment programs, including, but not limited to: Matcher, Needle, Stretcher, Water, Wordmatch, etc. (Rice, P., Longden, I. & Bleasby, A. EMBOSS: The European Molecular Biology Open Software Suite. Trends in Genetics 16(6) 276-77 (2000)). In an embodiment, the subject disclosure relates to calculating percent identity between two polynucleotides or amino acid sequences using the Ngila alignment program (Cartwright, R. Ngila: global pairwise alignments with logarithmic and affine gap costs. Bioinformatics. 23(11): 1427-28. Jun. 1, 2007). In an embodiment, the subject disclosure relates to calculating percent identity between two polynucleotides or amino acid sequences using the probA, also known as propA, alignment program (Mückstein, U., Hofacker, IL, & Stadler, PF. Stochastic pairwise alignments. Bioinformatics 18 Suppl. 2:S153-60. 2002). In an embodiment, the subject disclosure relates to calculating percent identity between two polynucleotides or amino acid sequences using the SEQALN suite of alignment programs (Hardy, P. & Waterman, M. The Sequence Alignment Software Library at USC. 1997). In an embodiment, the subject disclosure relates to calculating percent identity between two polynucleotides or amino acid sequences using the SIM suite of alignment programs, including, but not limited to, GAP, NAP, LAP, etc. (Huang, X & Miller, W. A Time-Efficient, Linear-Space Local Similarity Algorithm. Advances in Applied Mathematics, vol. 12 (1991) 337-57). In an embodiment, the subject disclosure relates to calculating percent identity between two polynucleotides or amino acid sequences using the UGENE alignment program (Okonechnikov, K., Golosova, O. & Fursov, M. Unipro UGENE: a unified bioinformatics toolkit. Bioinformatics. 2012 28:1166-67). In an embodiment, the subject disclosure relates to calculating percent identity between two polynucleotides or amino acid sequences using the BAli-Phy alignment program (Suchard, M A & Redelings, B D. BAli-Phy: simultaneous Bayesian inference of alignment and phylogeny. Bioinformatics. 22:2047-48. 2006). In an embodiment, the subject disclosure relates to calculating percent identity between two polynucleotides or amino acid sequences using the Base-By-Base alignment program (Brodie, R., et. al. Base-By-Base: Single nucleotide-level analysis of whole viral genome alignments, BMC Bioinformatics, 5, 96, 2004). In an embodiment, the subject disclosure relates to calculating percent identity between two polynucleotides or amino acid sequences using the DECIPHER alignment program (E S Wright (2015) “DECIPHER: harnessing local sequence context to improve protein multiple sequence alignment.” BMC Bioinformatics, doi:10.1186/s12859-015-0749-z.). In an embodiment, the subject disclosure relates to calculating percent identity between two polynucleotides or amino acid sequences using the FSA alignment program (Bradley, R K, et. al. (2009) Fast Statistical Alignment. PLOS Computational Biology. 5:e1000392). In an embodiment, the subject disclosure relates to calculating percent identity between two polynucleotides or amino acid sequences using the Geneious alignment program (Kearse, M., et. al. (2012). Geneious Basic: an integrated and extendable desktop software platform for the organization and analysis of sequence data. Bioinformatics, 28 (12), 1647-49). In an embodiment, the subject disclosure relates to calculating percent identity between two polynucleotides or amino acid sequences using the Kalign alignment program (Lassmann, T. & Sonnhammer, E. Kalign—an accurate and fast multiple sequence alignment algorithm. BMC Bioinformatics 2005 6:298). In an embodiment, the subject disclosure relates to calculating percent identity between two polynucleotides or amino acid sequences using the MAVID alignment program (Bray, N. & Pachter, L. MAVID: Constrained Ancestral Alignment of Multiple Sequences. Genome Res. 2004 April; 14(4): 693-99). In an embodiment, the subject disclosure relates to calculating percent identity between two polynucleotides or amino acid sequences using the MSA alignment program (Lipman, D J, et.al. A tool for multiple sequence alignment. Proc. Nat'l Acad. Sci. USA. 1989; 86:4412-15). In an embodiment, the subject disclosure relates to calculating percent identity between two polynucleotides or amino acid sequences using the MultAlin alignment program (Corpet, F., Multiple sequence alignment with hierarchial clustering. Nucl. Acids Res., 1988, 16(22), 10881-90). In an embodiment, the subject disclosure relates to calculating percent identity between two polynucleotides or amino acid sequences using the LAGAN or MLAGAN alignment programs (Brudno, et. al. LAGAN and Multi-LAGAN: efficient tools for large-scale multiple alignment of genomic DNA. Genome Research 2003 April; 13(4): 721-31). In an embodiment, the subject disclosure relates to calculating percent identity between two polynucleotides or amino acid sequences using the Opal alignment program (Wheeler, T. J., & Kececiouglu, J. D. Multiple alignment by aligning alignments. Proceedings of the 15^{th} ISCB conference on Intelligent Systems for Molecular Biology. Bioinformatics. 23, i559-68, 2007). In an embodiment, the subject disclosure relates to calculating percent identity between two polynucleotides or amino acid sequences using the PicXAA suite of programs, including, but not limited to, PicXAA, PicXAA-R, PicXAA-Web, etc. (Mohammad, S., Sahraeian, E. & Yoon, B. PicXAA: greedy probabilistic construction of maximum expected accuracy alignment of multiple sequences. Nucleic Acids Research. 38(15):4917-28. 2010). In an embodiment, the subject disclosure relates to calculating percent identity between two polynucleotides or amino acid sequences using the PSAlign alignment program (SZE, S.-H., Lu, Y., & Yang, Q. (2006) A polynomial time solvable formulation of multiple sequence alignment Journal of Computational Biology, 13, 309-19). In an embodiment, the subject disclosure relates to calculating percent identity between two polynucleotides or amino acid sequences using the StatAlign alignment program (Novák, Á., et.al. (2008) StatAlign: an extendable software package for joint Bayesian estimation of alignments and evolutionary trees. Bioinformatics, 24(20):2403-04). In an embodiment, the subject disclosure relates to calculating percent identity between two polynucleotides or amino acid sequences using the Gap alignment program of Needleman and Wunsch (Needleman and Wunsch, Journal of Molecular Biology 48:443-453, 1970). In an embodiment, the subject disclosure relates to calculating percent identity between two polynucleotides or amino acid sequences using the BestFit alignment program of Smith and Waterman (Smith and Waterman, Advances in Applied Mathematics, 2:482-489, 1981, Smith et al., Nucleic Acids Research 11:2205-2220, 1983). These programs produces biologically meaningful multiple sequence alignments of divergent sequences. The calculated best match alignments for the selected sequences are lined up so that identities, similarities, and differences can be seen.

The term “similarity” refers to a comparison between amino acid sequences, and takes into account not only identical amino acids in corresponding positions, but also functionally similar amino acids in corresponding positions. Thus similarity between polypeptide sequences indicates functional similarity, in addition to sequence similarity

The term “homology” is sometimes used to refer to the level of similarity between two or more nucleic acid or amino acid sequences in terms of percent of positional identity (i.e., sequence similarity or identity). Homology also refers to the concept of evolutionary relatedness, often evidenced by similar functional properties among different nucleic acids or proteins that share similar sequences.

As used herein, the term “variants” means substantially similar sequences. For nucleotide sequences, naturally occurring variants can be identified with the use of well-known molecular biology techniques, such as, for example, with polymerase chain reaction (PCR) and hybridization techniques as outlined herein.

For nucleotide sequences, a variant comprises a deletion and/or addition of one or more nucleotides at one or more internal sites within the native polynucleotide and/or a substitution of one or more nucleotides at one or more sites in the native polynucleotide. As used herein, a “native” nucleotide sequence comprises a naturally occurring nucleotide sequence. For nucleotide sequences, naturally occurring variants can be identified with the use of well-known molecular biology techniques, as, for example, with polymerase chain reaction (PCR) and hybridization techniques as outlined below. Variant nucleotide sequences also include synthetically derived nucleotide sequences, such as those generated, for example, by using site-directed mutagenesis. Generally, variants of a particular nucleotide sequence of the invention will have at least about 40%, 45%, 50% >, 55%, 60%, 65%, 70%, 75%, 80%, 85%, 90%, 91%, 92%, 93%, 94%, 95%, 96%, 97%, 98%, 99% or more sequence identity to that particular nucleotide sequence as determined by sequence alignment programs and parameters described elsewhere herein. A biologically active variant of a nucleotide sequence of the invention may differ from that sequence by as few as 1-15 nucleic acid residues, as few as 1-10, such as 6-10, as few as 5, as few as 4, 3, 2, or even 1 nucleic acid residue.

As used herein the term “operably linked” relates to a first nucleic acid sequence is operably linked with a second nucleic acid sequence when the first nucleic acid sequence is in a functional relationship with the second nucleic acid sequence. For instance, a promoter is operably linked with a coding sequence when the promoter affects the transcription or expression of the coding sequence. When recombinantly produced, operably linked nucleic acid sequences are generally contiguous and, where necessary to join two protein-coding regions, in the same reading frame. However, elements need not be contiguous to be operably linked.

As used herein, the term “promoter” refers to a region of DNA that generally is located upstream (towards the 5′ region of a gene) of a gene and is needed to initiate and drive transcription of the gene. A promoter may permit proper activation or repression of a gene that it controls. A promoter may contain specific sequences that are recognized by transcription factors. These factors may bind to a promoter DNA sequence, which results in the recruitment of RNA polymerase, an enzyme that synthesizes RNA from the coding region of the gene. The promoter generally refers to all gene regulatory elements located upstream of the gene, including, upstream promoters, 5′ UTR, introns, and leader sequences.

As used herein, the term “upstream-promoter” refers to a contiguous polynucleotide sequence that is sufficient to direct initiation of transcription. As used herein, an upstream-promoter encompasses the site of initiation of transcription with several sequence motifs, which include TATA Box, initiator sequence, TFIIB recognition elements and other promoter motifs (Jennifer, E. F. et al., (2002) Genes & Dev., 16:2583-2592). The upstream promoter provides the site of action to RNA polymerase II which is a multi-subunit enzyme with the basal or general transcription factors like, TFIIA, B, D, E, F and H. These factors assemble into a transcription pre initiation complex that catalyzes the synthesis of RNA from DNA template.

The activation of the upstream-promoter is done by the additional sequence of regulatory DNA sequence elements to which various proteins bind and subsequently interact with the transcription initiation complex to activate gene expression. These gene regulatory elements sequences interact with specific DNA-binding factors. These sequence motifs may sometimes be referred to as cis-elements. Such cis-elements, to which tissue-specific or development-specific transcription factors bind, individually or in combination, may determine the spatiotemporal expression pattern of a promoter at the transcriptional level. These cis-elements vary widely in the type of control they exert on operably linked genes. Some elements act to increase the transcription of operably-linked genes in response to environmental responses (e.g., temperature, moisture, and wounding). Other cis-elements may respond to developmental cues (e.g., germination, seed maturation, and flowering) or to spatial information (e.g., tissue specificity). See, for example, Langridge et al., (1989) Proc. Natl. Acad. Sci. USA 86:3219-23. These cis-elements are located at a varying distance from transcription start point, some cis-elements (called proximal elements) are adjacent to a minimal core promoter region while other elements can be positioned several kilobases upstream or downstream of the promoter (enhancers).

As used herein, the terms “5′ untranslated region” or “5′ UTR” is defined as the untranslated segment in the 5′ terminus of pre-mRNAs or mature mRNAs. For example, on mature mRNAs, a 5′ UTR typically harbors on its 5′ end a 7-methylguanosine cap and is involved in many processes such as splicing, polyadenylation, mRNA export towards the cytoplasm, identification of the 5′ end of the mRNA by the translational machinery, and protection of the mRNAs against degradation.

As used herein, the term “intron” refers to any nucleic acid sequence comprised in a gene (or expressed polynucleotide sequence of interest) that is transcribed but not translated. Introns include untranslated nucleic acid sequence within an expressed sequence of DNA, as well as the corresponding sequence in RNA molecules transcribed therefrom. A construct described herein can also contain sequences that enhance translation and/or mRNA stability such as introns. An example of one such intron is the first intron of gene II of the histone H3 variant of Arabidopsis thaliana or any other commonly known intron sequence. Introns can be used in combination with a promoter sequence to enhance translation and/or mRNA stability.

As used herein, the terms “transcription terminator” or “terminator” is defined as the transcribed segment in the 3′ terminus of pre-mRNAs or mature mRNAs. For example, longer stretches of DNA beyond “polyadenylation signal” site is transcribed as a pre-mRNA. This DNA sequence usually contains transcription termination signal for the proper processing of the pre-mRNA into mature mRNA.

As used herein, the term “3′ untranslated region” or “3′ UTR” is defined as the untranslated segment in a 3′ terminus of the pre-mRNAs or mature mRNAs. For example, on mature mRNAs this region harbors the poly-(A) tail and is known to have many roles in mRNA stability, translation initiation, and mRNA export. In addition, the 3′ UTR is considered to include the polyadenylation signal and transcription terminator.

As used herein, the term “polyadenylation signal” designates a nucleic acid sequence present in mRNA transcripts that allows for transcripts, when in the presence of a poly-(A) polymerase, to be polyadenylated on the polyadenylation site, for example, located 10 to 30 bases downstream of the poly-(A) signal. Many polyadenylation signals are known in the art and are useful for the present invention. An exemplary sequence includes AAUAAA and variants thereof, as described in Loke J., et al., (2005) Plant Physiology 138(3); 1457-1468.

A “DNA binding transgene” is a polynucleotide coding sequence that encodes a DNA binding protein. The DNA binding protein is subsequently able to bind to another molecule. A binding protein can bind to, for example, a DNA molecule (a DNA-binding protein), a RNA molecule (an RNA-binding protein), and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity. For example, zinc finger proteins have DNA-binding, RNA-binding, and protein-binding activity.

Examples of DNA binding proteins include; meganucleases, zinc fingers, CRISPRs, and TALEN binding domains that can be “engineered” to bind to a predetermined nucleotide sequence. Typically, the engineered DNA binding proteins (e.g., zinc fingers, CRISPRs, or TALENs) are proteins that are non-naturally occurring. Non-limiting examples of methods for engineering DNA-binding proteins are design and selection. A designed DNA binding protein is a protein not occurring in nature whose design/composition results principally from rational criteria. Rational criteria for design include application of substitution rules and computerized algorithms for processing information in a database storing information of existing ZFP, CRISPR, and/or TALEN designs and binding data. See, for example, U.S. Pat. Nos. 6,140,081; 6,453,242; and 6,534,261; see also WO 98/53058; WO 98/53059; WO 98/53060; WO 02/016536 and WO 03/016496 and U.S. Publication Nos. 20110301073, 20110239315 and 20119145940.

A “zinc finger DNA binding protein” (or binding domain) is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein or ZFP. Zinc finger binding domains can be “engineered” to bind to a predetermined nucleotide sequence. Non-limiting examples of methods for engineering zinc finger proteins are design and selection. A designed zinc finger protein is a protein not occurring in nature whose design/composition results principally from rational criteria. Rational criteria for design include application of substitution rules and computerized algorithms for processing information in a database storing information of existing ZFP designs and binding data. See, for example, U.S. Pat. Nos. 6,140,081; 6,453,242; 6,534,261 and 6,794,136; see also WO 98/53058; WO 98/53059; WO 98/53060; WO 02/016536 and WO 03/016496.

In other examples, the DNA-binding domain of one or more of the nucleases comprises a naturally occurring or engineered (non-naturally occurring) TAL effector DNA binding domain. See, e.g., U.S. Patent Publication No. 20110301073, incorporated by reference in its entirety herein. The plant pathogenic bacteria of the genus Xanthomonas are known to cause many diseases in important crop plants. Pathogenicity of Xanthomonas depends on a conserved type III secretion (T3S) system which injects more than different effector proteins into the plant cell. Among these injected proteins are transcription activator-like (TALEN) effectors which mimic plant transcriptional activators and manipulate the plant transcriptome (see Kay et al., (2007) Science 318:648-651). These proteins contain a DNA binding domain and a transcriptional activation domain. One of the most well characterized TAL-effectors is AvrBs3 from Xanthomonas campestgris pv. Vesicatoria (see Bonas et al., (1989) μMol Gen Genet 218: 127-136 and WO2010079430). TAL-effectors contain a centralized domain of tandem repeats, each repeat containing approximately 34 amino acids, which are key to the DNA binding specificity of these proteins. In addition, they contain a nuclear localization sequence and an acidic transcriptional activation domain (for a review see Schornack S, et al., (2006) J Plant Physiol 163(3): 256-272). In addition, in the phytopathogenic bacteria Ralstonia solanacearum two genes, designated brg11 and hpx17 have been found that are homologous to the AvrBs3 family of Xanthomonas in the R. solanacearum biovar strain GMI1000 and in the biovar 4 strain RS1000 (See Heuer et al., (2007) Appl and Enviro Micro 73(13): 4379-4384). These genes are 98.9% identical in nucleotide sequence to each other but differ by a deletion of 1,575 bp in the repeat domain of hpx17. However, both gene products have less than 40% sequence identity with AvrBs3 family proteins of Xanthomonas. See, e.g., U.S. Patent Publication No. 20110301073, incorporated by reference in its entirety.

Specificity of these TAL effectors depends on the sequences found in the tandem repeats. The repeated sequence comprises approximately 102 bp and the repeats are typically 91-100% homologous with each other (Bonas et al., ibid). Polymorphism of the repeats is usually located at positions 12 and 13 and there appears to be a one-to-one correspondence between the identity of the hypervariable diresidues at positions 12 and 13 with the identity of the contiguous nucleotides in the TAL-effector's target sequence (see Moscou and Bogdanove, (2009) Science 326:1501 and Boch et al., (2009) Science 326:1509-1512). Experimentally, the natural code for DNA recognition of these TAL-effectors has been determined such that an HD sequence at positions 12 and 13 leads to a binding to cytosine (C), NG binds to T, NI to A, C, G or T, NN binds to A or G, and ING binds to T. These DNA binding repeats have been assembled into proteins with new combinations and numbers of repeats, to make artificial transcription factors that are able to interact with new sequences and activate the expression of a non-endogenous reporter gene in plant cells (Boch et al., ibid). Engineered TAL proteins have been linked to a FokI cleavage half domain to yield a TAL effector domain nuclease fusion (TALEN) exhibiting activity in a yeast reporter assay (plasmid based target).

The CRISPR (Clustered Regularly Interspaced Short Palindromic Repeats)/Cas (CRISPR Associated) nuclease system is a recently engineered nuclease system based on a bacterial system that can be used for genome engineering. It is based on part of the adaptive immune response of many bacteria and Archaea. When a virus or plasmid invades a bacterium, segments of the invader's DNA are converted into CRISPR RNAs (crRNA) by the ‘immune’ response. This crRNA then associates, through a region of partial complementarity, with another type of RNA called tracrRNA to guide the Cas9 nuclease to a region homologous to the crRNA in the target DNA called a “protospacer.” Cas9 cleaves the DNA to generate blunt ends at the double-stranded break (DSB) at sites specified by a 20-nucleotide guide sequence contained within the crRNA transcript. Cas9 requires both the crRNA and the tracrRNA for site specific DNA recognition and cleavage. This system has now been engineered such that the crRNA and tracrRNA can be combined into one molecule (the “single guide RNA”), and the crRNA equivalent portion of the single guide RNA can be engineered to guide the Cas9 nuclease to target any desired sequence (see Jinek et al., (2012) Science 337, pp. 816-821, Jinek et al., (2013), eLife 2:e00471, and David Segal, (2013) eLife 2:e00563). In other examples, the crRNA associates with the tracrRNA to guide the Cpf1 nuclease to a region homologous to the crRNA to cleave DNA with staggered ends (see Zetsche, Bernd, et al. Cell 163.3 (2015): 759-771.). Thus, the CRISPR/Cas system can be engineered to create a DSB at a desired target in a genome, and repair of the DSB can be influenced by the use of repair inhibitors to cause an increase in error prone repair.

In other examples, the DNA binding transgene/heterologous coding sequence is a site specific nuclease that comprises an engineered (non-naturally occurring) Meganuclease (also described as a homing endonuclease). The recognition sequences of homing endonucleases or meganucleases such as I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII are known. See also U.S. Pat. Nos. 5,420,032; 6,833,252; Belfort et al., (1997) Nucleic Acids Res. 25:3379-30 3388; Dujon et al., (1989) Gene 82:115-118; Perler et al., (1994) Nucleic Acids Res. 22, 11127; Jasin (1996) Trends Genet. 12:224-228; Gimble et al., (1996) J. Mol. Biol. 263:163-180; Argast et al., (1998) J. Mol. Biol. 280:345-353 and the New England Biolabs catalogue. In addition, the

DNA-binding specificity of homing endonucleases and meganucleases can be engineered to bind non-natural target sites. See, for example, Chevalier et al., (2002) Molec. Cell 10:895-905; Epinat et al., (2003) Nucleic Acids Res. 5 31:2952-2962; Ashworth et al., (2006) Nature 441:656-659; Paques et al., (2007) Current Gene Therapy 7:49-66; U.S. Patent Publication No. 20070117128. The DNA-binding domains of the homing endonucleases and meganucleases may be altered in the context of the nuclease as a whole (i.e., such that the nuclease includes the cognate cleavage domain) or may be fused to a heterologous cleavage domain.

As used herein, the term “transformation” encompasses all techniques that a nucleic acid molecule can be introduced into such a cell. Examples include, but are not limited to: transfection with viral vectors; transformation with plasmid vectors; electroporation; lipofection; microinjection (Mueller et al., (1978) Cell 15:579-85); Agrobacterium-mediated transfer; direct DNA uptake; WHISKERS™-mediated transformation; and microprojectile bombardment. These techniques may be used for both stable transformation and transient transformation of a plant cell. “Stable transformation” refers to the introduction of a nucleic acid fragment into a genome of a host organism resulting in genetically stable inheritance. Once stably transformed, the nucleic acid fragment is stably integrated in the genome of the host organism and any subsequent generation. Host organisms containing the transformed nucleic acid fragments are referred to as “transgenic” organisms. “Transient transformation” refers to the introduction of a nucleic acid fragment into the nucleus, or DNA-containing organelle, of a host organism resulting in gene expression without genetically stable inheritance.

An exogenous nucleic acid sequence. In one example, a transgene/heterologous coding sequence is a gene sequence (e.g., an herbicide-resistance gene), a gene encoding an industrially or pharmaceutically useful compound, or a gene encoding a desirable agricultural trait. In yet another example, the transgene/heterologous coding sequence is an antisense nucleic acid sequence, wherein expression of the antisense nucleic acid sequence inhibits expression of a target nucleic acid sequence. A transgene/heterologous coding sequence may contain regulatory sequences operably linked to the transgene/heterologous coding sequence (e.g., a promoter). In some embodiments, a polynucleotide sequence of interest is a transgene. However, in other embodiments, a polynucleotide sequence of interest is an endogenous nucleic acid sequence, wherein additional genomic copies of the endogenous nucleic acid sequence are desired, or a nucleic acid sequence that is in the antisense orientation with respect to the sequence of a target nucleic acid molecule in the host organism.

As used herein, the term a transgenic “event” is produced by transformation of plant cells with heterologous DNA, i.e., a nucleic acid construct that includes a transgene/heterologous coding sequence of interest, regeneration of a population of plants resulting from the insertion of the transgene/heterologous coding sequence into the genome of the plant, and selection of a particular plant characterized by insertion into a particular genome location. The term “event” refers to the original transformant and progeny of the transformant that include the heterologous DNA. The term “event” also refers to progeny produced by a sexual outcross between the transformant and another variety that includes the genomic/transgene DNA. Even after repeated back-crossing to a recurrent parent, the inserted transgene/heterologous coding sequence DNA and flanking genomic DNA (genomic/transgene DNA) from the transformed parent is present in the progeny of the cross at the same chromosomal location. The term “event” also refers to DNA from the original transformant and progeny thereof comprising the inserted DNA and flanking genomic sequence immediately adjacent to the inserted DNA that would be expected to be transferred to a progeny that receives inserted DNA including the transgene/heterologous coding sequence of interest as the result of a sexual cross of one parental line that includes the inserted DNA (e.g., the original transformant and progeny resulting from selfing) and a parental line that does not contain the inserted DNA.

As used herein, the terms “Polymerase Chain Reaction” or “PCR” define a procedure or technique in which minute amounts of nucleic acid, RNA and/or DNA, are amplified as described in U.S. Pat. No. 4,683,195 issued Jul. 28, 1987. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage or plasmid sequences, etc. See generally Mullis et al., Cold Spring Harbor Symp. Quant. Biol., 51:263 (1987); Erlich, ed., PCR Technology, (Stockton Press, NY, 1989).

As used herein, the term “primer” refers to an oligonucleotide capable of acting as a point of initiation of synthesis along a complementary strand when conditions are suitable for synthesis of a primer extension product. The synthesizing conditions include the presence of four different deoxyribonucleotide triphosphates and at least one polymerization-inducing agent such as reverse transcriptase or DNA polymerase. These are present in a suitable buffer, which may include constituents which are co-factors or which affect conditions such as pH and the like at various suitable temperatures. A primer is preferably a single strand sequence, such that amplification efficiency is optimized, but double stranded sequences can be utilized.

As used herein, the term “probe” refers to an oligonucleotide that hybridizes to a target sequence. In the TaqMan® or TaqMan®-style assay procedure, the probe hybridizes to a portion of the target situated between the annealing site of the two primers. A probe includes about eight nucleotides, about ten nucleotides, about fifteen nucleotides, about twenty nucleotides, about thirty nucleotides, about forty nucleotides, or about fifty nucleotides. In some embodiments, a probe includes from about eight nucleotides to about fifteen nucleotides. A probe can further include a detectable label, e.g., a fluorophore (Texas-Red®, Fluorescein isothiocyanate, etc.,). The detectable label can be covalently attached directly to the probe oligonucleotide, e.g., located at the probe's 5′ end or at the probe's 3′ end. A probe including a fluorophore may also further include a quencher, e.g., Black Hole Quencher™, Iowa Black™, etc.

As used herein, the terms “restriction endonucleases” and “restriction enzymes” refer to bacterial enzymes, each of which cut double-stranded DNA at or near a specific nucleotide sequence. Type-2 restriction enzymes recognize and cleave DNA at the same site, and include but are not limited to XbaI, BamHI, HindIII, EcoRI, XhoI, SalI, KpnI, AvaI, PstI and SmaI.

As used herein, the term “vector” is used interchangeably with the terms “construct”, “cloning vector” and “expression vector” and means the vehicle by which a DNA or RNA sequence (e.g. a foreign gene) can be introduced into a host cell, so as to transform the host and promote expression (e.g. transcription and translation) of the introduced sequence. A “non-viral vector” is intended to mean any vector that does not comprise a virus or retrovirus. In some embodiments a “vector” is a sequence of DNA comprising at least one origin of DNA replication and at least one selectable marker gene. Examples include, but are not limited to, a plasmid, cosmid, bacteriophage, bacterial artificial chromosome (BAC), or virus that carries exogenous DNA into a cell. A vector can also include one or more genes, antisense molecules, and/or selectable marker genes and other genetic elements known in the art. A vector may transduce, transform, or infect a cell, thereby causing the cell to express the nucleic acid molecules and/or proteins encoded by the vector.

The term “plasmid” defines a circular strand of nucleic acid capable of autosomal replication in either a prokaryotic or a eukaryotic host cell. The term includes nucleic acid which may be either DNA or RNA and may be single- or double-stranded. The plasmid of the definition may also include the sequences which correspond to a bacterial origin of replication.

As used herein, the term “selectable marker gene” as used herein defines a gene or other expression cassette which encodes a protein which facilitates identification of cells into which the selectable marker gene is inserted. For example a “selectable marker gene” encompasses reporter genes as well as genes used in plant transformation to, for example, protect plant cells from a selective agent or provide resistance/tolerance to a selective agent. In one embodiment only those cells or plants that receive a functional selectable marker are capable of dividing or growing under conditions having a selective agent. The phrase “marker-positive” refers to plants that have been transformed to include a selectable marker gene.

As used herein, the term “detectable marker” refers to a label capable of detection, such as, for example, a radioisotope, fluorescent compound, bioluminescent compound, a chemiluminescent compound, metal chelator, or enzyme. Examples of detectable markers include, but are not limited to, the following: fluorescent labels (e.g., FITC, rhodamine, lanthanide phosphors), enzymatic labels (e.g., horseradish peroxidase, β-galactosidase, luciferase, alkaline phosphatase), chemiluminescent, biotinyl groups, predetermined polypeptide epitopes recognized by a secondary reporter (e.g., leucine zipper pair sequences, binding sites for secondary antibodies, metal binding domains, epitope tags). In an embodiment, a detectable marker can be attached by spacer arms of various lengths to reduce potential steric hindrance.

As used herein, the terms “cassette”, “expression cassette” and “gene expression cassette” refer to a segment of DNA that can be inserted into a nucleic acid or polynucleotide at specific restriction sites or by homologous recombination. As used herein the segment of DNA comprises a polynucleotide that encodes a polypeptide of interest, and the cassette and restriction sites are designed to ensure insertion of the cassette in the proper reading frame for transcription and translation. In an embodiment, an expression cassette can include a polynucleotide that encodes a polypeptide of interest and having elements in addition to the polynucleotide that facilitate transformation of a particular host cell. In an embodiment, a gene expression cassette may also include elements that allow for enhanced expression of a polynucleotide encoding a polypeptide of interest in a host cell. These elements may include, but are not limited to: a promoter, a minimal promoter, an enhancer, a response element, a terminator sequence, a polyadenylation sequence, and the like.

As used herein a “linker” or “spacer” is a bond, molecule or group of molecules that binds two separate entities to one another. Linkers and spacers may provide for optimal spacing of the two entities or may further supply a labile linkage that allows the two entities to be separated from each other. Labile linkages include photocleavable groups, acid-labile moieties, base-labile moieties and enzyme-cleavable groups. The terms “polylinker” or “multiple cloning site” as used herein defines a cluster of three or more Type-2 restriction enzyme sites located within 10 nucleotides of one another on a nucleic acid sequence. In other instances the term “polylinker” as used herein refers to a stretch of nucleotides that are targeted for joining two sequences via any known seamless cloning method (i.e., Gibson Assembly®, NEBuilder HiFID NA Assembly®, Golden Gate Assembly, BioBrick® Assembly, etc.). Constructs comprising a polylinker are utilized for the insertion and/or excision of nucleic acid sequences such as the coding region of a gene.

As used herein, the term “control” refers to a sample used in an analytical procedure for comparison purposes. A control can be “positive” or “negative”. For example, where the purpose of an analytical procedure is to detect a differentially expressed transcript or polypeptide in cells or tissue, it is generally preferable to include a positive control, such as a sample from a known plant exhibiting the desired expression, and a negative control, such as a sample from a known plant lacking the desired expression.

As used herein, the term “plant” includes a whole plant and any descendant, cell, tissue, or part of a plant. A class of plant that can be used in the present invention is generally as broad as the class of higher and lower plants amenable to mutagenesis including angiosperms (monocotyledonous and dicotyledonous plants), gymnosperms, ferns and multicellular algae. Thus, “plant” includes dicot and monocot plants. The term “plant parts” include any part(s) of a plant, including, for example and without limitation: seed (including mature seed and immature seed); a plant cutting; a plant cell; a plant cell culture; a plant organ (e.g., pollen, embryos, flowers, fruits, shoots, leaves, roots, stems, and explants). A plant tissue or plant organ may be a seed, protoplast, callus, or any other group of plant cells that is organized into a structural or functional unit. A plant cell or tissue culture may be capable of regenerating a plant having the physiological and morphological characteristics of the plant from which the cell or tissue was obtained, and of regenerating a plant having substantially the same genotype as the plant. In contrast, some plant cells are not capable of being regenerated to produce plants. Regenerable cells in a plant cell or tissue culture may be embryos, protoplasts, meristematic cells, callus, pollen, leaves, anthers, roots, root tips, silk, flowers, kernels, ears, cobs, husks, or stalks.

Plant parts include harvestable parts and parts useful for propagation of progeny plants. Plant parts useful for propagation include, for example and without limitation: seed; fruit; a cutting; a seedling; a tuber; and a rootstock. A harvestable part of a plant may be any useful part of a plant, including, for example and without limitation: flower; pollen; seedling; tuber; leaf; stem; fruit; seed; and root.

A plant cell is the structural and physiological unit of the plant, comprising a protoplast and a cell wall. A plant cell may be in the form of an isolated single cell, or an aggregate of cells (e.g., a friable callus and a cultured cell), and may be part of a higher organized unit (e.g., a plant tissue, plant organ, and plant). Thus, a plant cell may be a protoplast, a gamete producing cell, or a cell or collection of cells that can regenerate into a whole plant. As such, a seed, which comprises multiple plant cells and is capable of regenerating into a whole plant, is considered a “plant cell” in embodiments herein.

As used herein, the term “small RNA” refers to several classes of non-coding ribonucleic acid (ncRNA). The term small RNA describes the short chains of ncRNA produced in bacterial cells, animals, plants, and fungi. These short chains of ncRNA may be produced naturally within the cell or may be produced by the introduction of an exogenous sequence that expresses the short chain or ncRNA. The small RNA sequences do not directly code for a protein, and differ in function from other RNA in that small RNA sequences are only transcribed and not translated. The small RNA sequences are involved in other cellular functions, including gene expression and modification. Small RNA molecules are usually made up of about 20 to 30 nucleotides. The small RNA sequences may be derived from longer precursors. The precursors form structures that fold back on each other in self-complementary regions; they are then processed by the nuclease Dicer in animals or DCL1 in plants.

Many types of small RNA exist either naturally or produced artificially, including microRNAs (miRNAs), short interfering RNAs (siRNAs), antisense RNA, short hairpin RNA (shRNA), and small nucleolar RNAs (snoRNAs). Certain types of small RNA, such as microRNA and siRNA, are important in gene silencing and RNA interference (RNAi). Gene silencing is a process of genetic regulation in which a gene that would normally be expressed is “turned off” by an intracellular element, in this case, the small RNA. The protein that would normally be formed by this genetic information is not formed due to interference, and the information coded in the gene is blocked from expression.

As used herein, the term “small RNA” encompasses RNA molecules described in the literature as “tiny RNA” (Storz, (2002) Science 296:1260-3; Illangasekare et al., (1999) RNA 5:1482-1489); prokaryotic “small RNA” (sRNA) (Wassarman et al., (1999) Trends Microbiol. 7:37-45); eukaryotic “noncoding RNA (ncRNA)”; “micro-RNA (miRNA)”; “small non-mRNA (snmRNA)”; “functional RNA (fRNA)”; “transfer RNA (tRNA)”; “catalytic RNA” [e.g., ribozymes, including self-acylating ribozymes (Illangaskare et al., (1999) RNA 5:1482-1489); “small nucleolar RNAs (snoRNAs),” “tmRNA” (a.k.a. “10S RNA,” Muto et al., (1998) Trends Biochem Sci. 23:25-29; and Gillet et al., (2001) Mol Microbiol. 42:879-885); RNAi molecules including without limitation “small interfering RNA (siRNA),” “endoribonuclease-prepared siRNA (e-siRNA),” “short hairpin RNA (shRNA),” and “small temporally regulated RNA (stRNA),” “diced siRNA (d-siRNA),” and aptamers, oligonucleotides and other synthetic nucleic acids that comprise at least one uracil base.

Unless otherwise specifically explained, all technical and scientific terms used herein have the same meaning as commonly understood by those of ordinary skill in the art to which this disclosure belongs. Definitions of common terms in molecular biology can be found in, for example: Lewin, Genes IV, Oxford University Press, 1994 (ISBN 0-19-854287-9); Kendrew et al. (eds.), The Encyclopedia of Molecular Biology, Blackwell Science Ltd., 1994 (ISBN 0-632-02182-9); and Meyers (ed.), Molecular Biology and Biotechnology: A Comprehensive Desk Reference, VCH Publishers, Inc., 1995 (ISBN 1-56081-569-8).

### Embodiments

Provided are methods and compositions for using a cis-acting regulatory element within a chimeric regulatory molecule to express a heterologous coding sequence within a plant. In an embodiment, a cis-acting regulatory element can comprise SEQ ID NO:9, SEQ ID NOs:25-583, SEQ ID NOs:585-2268. In another embodiment, a cis-acting regulatory element shares 80%, 85%, 90%, 91%, 92%, 93%, 94%, 95%, 96%, 97%, 98%, 99%, 99.5%, 99.8%, or 100% identity to SEQ ID NO:9, SEQ ID NOs:25-583, SEQ ID NOs:585-2268. In other embodiments, the chimeric regulatory molecule comprises a promoter, a 5′ UTR or an intron operably linked to the cis-acting regulatory element. In some embodiments the cis-acting regulatory element is provided as a multiple copy within the chimeric regulatory molecule. In further embodiments, the chimeric regulatory molecule is operably linked to a heterologous coding sequence/transgene to produce a gene expression cassette. In additional embodiments, the cis-acting regulatory element modulates expression of the heterologous coding sequence to either enhance or reduce expression of the heterologous coding sequence/transgene.

In an embodiment, an isolated polynucleotide is provided comprising a cis-acting regulatory element, wherein the cis-acting regulatory element is at least 80%, 85%, 90%, 91%, 92%, 93%, 94%, 95%, 96%, 97%, 98%, 99%, 99.5%, 99.8%, or 100% identical to SEQ ID NO:9, SEQ ID NOs:25-583, SEQ ID NOs:585-2268. In an embodiment, a chimeric regulatory molecule comprises a cis-acting regulatory element comprising a polynucleotide of at least 80%, 85%, 90%, 91%, 92%, 93%, 94%, 95%, 96%, 97%, 98%, 99%, 99.5%, 99.8%, or 100% identity to the polynucleotide SEQ ID NO:9, SEQ ID NOs:25-583, SEQ ID NOs:585-2268. In an embodiment, an isolated polynucleotide is provided comprising at least 80%, 85%, 90%, 91%, 92%, 93%, 94%, 95%, 96%, 97%, 98%, 99%, 99.5%, 99.8%, or 100% identity to the polynucleotide of SEQ ID NO:9, SEQ ID NOs:25-583, SEQ ID NOs:585-2268. In an embodiment, a nucleic acid vector is provided comprising a cis-acting regulatory element that is at least 80%, 85%, 90%, 91%, 92%, 93%, 94%, 95%, 96%, 97%, 98%, 99%, 99.5%, 99.8%, or 100% SEQ ID NO:9, SEQ ID NOs:25-583,SEQ ID NOs:585-2268. In an embodiment, a chimeric regulatory molecule is provided comprising a cis-acting regulatory element that is at least 80%, 85%, 90%, 91%, 92%, 93%, 94%, 95%, 96%, 97%, 98%, 99%, 99.5%, 99.8%, or 100% identical SEQ ID NO:9, SEQ ID NOs: 25-583, SEQ ID NOs:585-2268 wherein the polynucleotide is operably linked to a chimeric regulatory molecule. In an embodiment, a chimeric regulatory molecule is provided comprising a cis-acting regulatory element is at least 80%, 85%, 90%, 91%, 92%, 93%, 94%, 95%, 96%, 97%, 98%, 99%, 99.5%, 99.8%, or 100% identical to SEQ ID NO:9, SEQ ID NOs:25-583, SEQ ID NOs:585-2268 that is operably linked to an intron. In an embodiment, a chimeric regulatory molecule is provided comprising a cis-acting regulatory element is at least 80%, 85%, 90%, 91%, 92%, 93%, 94%, 95%, 96%, 97%, 98%, 99%, 99.5%, 99.8%, or 100% identical to SEQ ID NO:9, SEQ ID NOs:25-583, SEQ ID NOs:585-2268 that is operably linked to a 5′UTR. In an embodiment, a chimeric regulatory molecule is provided comprising a cis-acting regulatory element is at least 80%, 85%, 90%, 91%, 92%, 93%, 94%, 95%, 96%, 97%, 98%, 99%, 99.5%, 99.8%, or 100% identical to SEQ ID NO:9, SEQ ID NOs:25-583, SEQ ID NOs:585-2268 that is operably linked to a polylinker. In an embodiment, a gene expression cassette is provided comprising a cis-acting regulatory element is at least 80%, 85%, 90%, 91%, 92%, 93%, 94%, 95%, 96%, 97%, 98%, 99%, 99.5%, 99.8%, or 100% identical to SEQ ID NO:9, SEQ ID NOs:25-583, SEQ ID NOs:585-2268. In an embodiment, a gene expression cassette is provided comprising a chimeric regulatory molecule element that is at least 80%, 85%, 90%, 91%, 92%, 93%, 94%, 95%, 96%, 97%, 98%, 99%, 99.5%, 99.8%, or 100% identical to SEQ ID NO:9, SEQ ID NOs:25-583, SEQ ID NOs:585-2268. In some instances the gene expression cassette comprising a cis-acting regulatory element is at least 80%, 85%, 90%, 91%, 92%, 93%, 94%, 95%, 96%, 97%, 98%, 99%, 99.5%, 99.8%, or 100% identical to SEQ ID NO:9, SEQ ID NOs:25-583, SEQ ID NOs:585-2268 further comprises a promoter, a 5′UTR, an intron, a polylinker, a heterologous coding sequence or a transgene. In an embodiment, a vector is provided comprising a cis-acting regulatory element is at least 80%, 85%, 90%, 91%, 92%, 93%, 94%, 95%, 96%, 97%, 98%, 99%, 99.5%, 99.8%, or 100% identical to SEQ ID NO:9, SEQ ID NOs:25-583, SEQ ID NOs:585-2268. In an embodiment, a vector is provided comprising a chimeric regulatory molecule element that is at least 80%, 85%, 90%, 91%, 92%, 93%, 94%, 95%, 96%, 97%, 98%, 99%, 99.5%, 99.8%, or 100% identical to SEQ ID NO:9, SEQ ID NOs:25-583, SEQ ID NOs:585-2268. In some instances the vector comprising a cis-acting regulatory element is at least 80%, 85%, 90%, 91%, 92%, 93%, 94%, 95%, 96%, 97%, 98%, 99%, 99.5%, 99.8%, or 100% identical to SEQ ID NO:9, SEQ ID NOs:25-583, SEQ ID NOs:585-2268 further comprises a promoter, a 5′UTR, an intron, a polylinker, a heterologous coding sequence or a transgene.

In an embodiment, the chimeric regulatory molecule comprises at least one copy of a cis-acting regulatory element that shares at least 80%, 85%, 90%, 91%, 92%, 93%, 94%, 95%, 96%, 97%, 98%, 99%, 99.5%, 99.8%, or 100% identical to SEQ ID NO:9, SEQ ID NOs:25-583, SEQ ID NOs:585-2268. In other embodiments, the cis-acting regulatory element is provided in the chimeric regulatory molecule as single, double, triple, quadruple copy. In additional embodiments, the cis-acting regulatory element is provided in the chimeric regulatory molecule in multiple copies; for example 1-100 copies. In further embodiments the multiple copies of the cis-acting regulatory element may be sequentially linked to one another. In other embodiments the multiple copies of the cis-acting regulatory element may be separated from one another by an intervening sequence. Such an intervening sequence may be any length, for example from 1 bp to 10,000 bp in length.

In an embodiment, chimeric regulatory molecule comprising a cis-acting regulatory element is at least 80%, 85%, 90%, 91%, 92%, 93%, 94%, 95%, 96%, 97%, 98%, 99%, 99.5%, 99.8%, or 100% identical to SEQ ID NO:9, SEQ ID NOs:25-583, SEQ ID NOs:585-2268 is operably linked to a transgene, wherein the transgene/heterologous coding sequence can be an insecticidal resistance transgene, an herbicide tolerance transgene, a nitrogen use efficiency transgene, a water use efficiency transgene, a nutritional quality transgene, a DNA binding transgene, a small RNA transgene, selectable marker transgene, or combinations thereof.

In an embodiment, a nucleic acid vector comprises a gene expression cassette as disclosed herein. In an embodiment, a vector can be a plasmid, a cosmid, a bacterial artificial chromosome (BAC), a bacteriophage, a virus, or an excised polynucleotide fragment for use in direct transformation or gene targeting such as a donor DNA.

Another aspect of the subject disclosure comprises a functional variant which differs in one or more nucleotides from those of the nucleotide sequences comprising the regulatory element, provided herein. Such a variant is produced as the result of one or more modifications (e.g., deletion, rearrangement, or insertion) of the nucleotide sequences comprising the sequence described herein. For example, fragments and variants of SEQ ID NO:9, SEQ ID NOs:25-583, SEQ ID NOs:585-2268 may be fused with a regulatory element to produce a chimeric regulatory element. This chimeric regulatory element can be used in a DNA construct or in a gene expression cassette to drive expression of a heterologous coding sequence. As used herein, the term “fragment” refers to a portion of the nucleic acid sequence. Fragments of cis-acting regulatory elements of SEQ ID NO:9, SEQ ID NOs:25-583, SEQ ID NOs:585-2268 may retain the biological activity of modulating expression by initiating transcription to drive enhanced expression. Fragments of a nucleotide sequence for the cis-acting regulatory elements of SEQ ID NO:9, SEQ ID NOs:25-583, SEQ ID NOs:585-2268 may range from at least about 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 nucleotides, 31 nucleotides, 32 nucleotides, 33 nucleotides, or up to the full-length nucleotide sequence of the cis-acting regulatory element.

A biologically active portion of a cis-acting regulatory element of SEQ ID NO:9, SEQ ID NOs:25-583, SEQ ID NOs:585-2268 can be prepared by isolating a portion of the cis-acting regulatory elements of SEQ ID NO:9, SEQ ID NOs:25-583, SEQ ID NOs:585-2268, and assessing the biological activity of the portion of the cis-acting regulatory element to modulate transcription of a heterologous coding sequence or a transgene. Nucleic acid molecules that are fragments of a cis-acting regulatory element of SEQ ID NO:9, SEQ ID NOs:25-583, SEQ ID NOs:585-2268 comprise at least about 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 nucleotides, 31 nucleotides, 32 nucleotides, 33 nucleotides, or up to the full-length nucleotide sequence of the cis-acting regulatory element of SEQ ID NO:9, SEQ ID NOs:25-583, SEQ ID NOs:585-2268 as disclosed herein.

A biologically active portion of a cis-acting regulatory element of SEQ ID NO:9, SEQ ID NOs:25-583, SEQ ID NOs:585-2268 can be prepared by isolating a portion of the cis-acting regulatory elements of SEQ ID NO:9, SEQ ID NOs:25-583, SEQ ID NOs:585-2268, and assessing the biological activity of the portion of the cis-acting regulatory element to enhance expression of a heterologous coding sequence or a transgene. Nucleic acid molecules that are fragments of a cis-acting regulatory element of SEQ ID NO:9, SEQ ID NOs:25-583, SEQ ID NOs:585-2268 comprise at least about 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 nucleotides, 31 nucleotides, 32 nucleotides, 33 nucleotides, or up to the full-length nucleotide sequence of the cis-acting regulatory element of SEQ ID NO:9, SEQ ID NOs:25-583, SEQ ID NOs:585-2268 as disclosed herein.

Variant nucleotide sequences also encompass sequences derived from a mutagenic and recombinogenic procedure such as DNA shuffling. With such a procedure, the cis-acting regulatory elements of SEQ ID NO:9, SEQ ID NOs:25-583, SEQ ID NOs:585-2268 can be manipulated to create a new cis-acting regulatory element. In this manner, libraries of recombinant polynucleotides are generated from a population of related sequence polynucleotides comprising sequence regions that have substantial sequence identity and can be homologously recombined in vitro or in vivo. Strategies for such DNA shuffling are known in the art. See, for example, Stemmer (1994) Proc. Natl. Acad. Sci. USA i: 10747-10751; Stemmer (1994) Nature 570:389-391; Crameri et al. (1997) Nature Biotech. 75:436-438; Moore et al. (1997) J. Mol. Biol. 272:336-347; Zhang et al. (1997) Proc. Natl. Acad. Sci. USA £4:4504-4509; Crameri et al. (1998) Nature 527:288-291; and U.S. Pat. Nos. 5,605,793 and 5,837,458.

The nucleotide sequences of the subject disclosure can be used to isolate corresponding sequences from other organisms, particularly other plants, more particularly other monocots. In this manner, methods such as PCR, hybridization, and the like can be used to identify such sequences based on their sequence homology to the sequences set forth herein. Sequences isolated based on their sequence identity to the entire cis-acting regulatory element of SEQ ID NO:9, SEQ ID NOs:25-583, SEQ ID NOs:585-2268 set forth herein or to fragments thereof are encompassed by the present invention.

In a PCR approach, oligonucleotide primers can be designed for use in PCR reactions to amplify corresponding DNA sequences from genomic DNA extracted from any plant of interest. Methods for designing PCR primers and PCR cloning are generally known in the art and are disclosed in Sambrook et al. (1989) Molecular Cloning: A Laboratory Manual (2d ed., Cold Spring Harbor Laboratory Press, Plainview, New York), hereinafter Sambrook. See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, and the like.

In hybridization techniques, all or part of a known nucleotide sequence is used as a probe that selectively hybridizes to other corresponding nucleotide sequences present in a population of cloned genomic DNA fragments from a chosen organism. The hybridization probes may be labeled with a detectable group such as P³² or any other detectable marker. Thus, for example, probes for hybridization can be made by labeling synthetic oligonucleotides based on the cis-acting regulatory element of SEQ ID NO:9, SEQ ID NOs:25-583, SEQ ID NOs:585-2268 of the subject disclosure. Methods for preparation of probes for hybridization and for construction of genomic libraries are generally known in the art and are disclosed in Sambrook. For example, the entire the cis-acting regulatory element of SEQ ID NO:9, SEQ ID NOs:25-583, SEQ ID NOs:585-2268 disclosed herein, or one or more portions thereof, may be used as a probe capable of specifically hybridizing to corresponding cis-acting regulatory elements. To achieve specific hybridization under a variety of conditions, such probes include sequences that are unique among the cis-acting regulatory element of SEQ ID NO:9, SEQ ID NOs:25-583, SEQ ID NOs:585-2268 and are at least about 10 nucleotides in length or at least about 20 nucleotides in length. Such probes may be used to amplify corresponding the cis-acting regulatory element from a chosen plant by PCR. This technique may be used to isolate additional coding sequences from a desired organism, or as a diagnostic assay to determine the presence of coding sequences in an organism. Hybridization techniques include hybridization screening of plated DNA libraries (either plaques or colonies; see, for example, Sambrook).

In accordance with one embodiment the nucleic acid vector further comprises a sequence encoding a selectable maker. In accordance with one embodiment the recombinant gene cassette is operably linked to an Agrobacterium T-DNA border. In accordance with one embodiment the recombinant gene cassette further comprises a first and second T-DNA border, wherein the first T-DNA border is operably linked to one end of a gene construct, and the second T-DNA border is operably linked to the other end of a gene construct. The first and second Agrobacterium T-DNA borders can be independently selected from T-DNA border sequences originating from bacterial strains selected from the group consisting of a nopaline synthesizing Agrobacterium T-DNA border, an ocotopine synthesizing Agrobacterium T-DNA border, a mannopine synthesizing Agrobacterium T-DNA border, a succinamopine synthesizing Agrobacterium T-DNA border, or any combination thereof. In one embodiment an Agrobacterium strain selected from the group consisting of a nopaline synthesizing strain, a mannopine synthesizing strain, a succinamopine synthesizing strain, or an octopine synthesizing strain is provided, wherein said strain comprises a plasmid wherein the plasmid comprises a transgene/heterologous coding sequence operably linked to a sequence selected from a cis-acting regulatory element that is at least 80%, 85%, 90%, 91%, 92%, 93%, 94%, 95%, 96%, 97%, 98%, 99%, 99.5%, 99.8%, or 100% identical to SEQ ID NO:9, SEQ ID NOs:25-583, SEQ ID NOs:585-2268. In another embodiment, the first and second Agrobacterium T-DNA borders can be independently selected from T-DNA border sequences originating from bacterial strains selected from the group consisting of a nopaline synthesizing Agrobacterium T-DNA border, an ocotopine synthesizing Agrobacterium T-DNA border, a mannopine synthesizing Agrobacterium T-DNA border, a succinamopine synthesizing Agrobacterium T-DNA border, or any combination thereof. In an embodiment an Agrobacterium strain selected from the group consisting of a nopaline synthesizing strain, a mannopine synthesizing strain, a succinamopine synthesizing strain, or an octopine synthesizing strain is provided, wherein said strain comprises a plasmid wherein the plasmid comprises a transgene/heterologous coding sequence operably linked to a sequence selected from a cis-acting regulatory element or a chimeric regulatory molecule that is at least 80%, 85%, 90%, 91%, 92%, 93%, 94%, 95%, 96%, 97%, 98%, 99%, 99.5%, 99.8%, or 100% identical to SEQ ID NO:9, SEQ ID NOs:25-583, SEQ ID NOs:585-2268.

### Traits For Introgression

In some embodiments a chimeric regulatory element comprising SEQ ID NO:9, SEQ ID NOs:25-583, SEQ ID NOs:585-2268 can be used to drive the expression of a heterologous coding sequence (for example, a transgene of interest) in a plant.

Transgenes of interest may be expressed by a chimeric regulatory element comprising SEQ ID NO:9, SEQ ID NOs:25-583, SEQ ID NOs:585-2268 of the subject disclosure. Exemplary transgenes of interest that are suitable for use in the present disclosed constructs include, but are not limited to, coding sequences that confer (1) resistance to pests or disease, (2) tolerance to herbicides, (3) value added agronomic traits, such as; yield improvement, nitrogen use efficiency, water use efficiency, and nutritional quality, (4) binding of a protein to DNA in a site specific manner, (5) expression of small RNA, and (6) selectable markers. In accordance with one embodiment, the chimeric regulatory element comprising SEQ ID NO:9, SEQ ID NOs:25-583, SEQ ID NOs:585-2268 is used to drive expression of a transgene/heterologous coding sequence encoding a selectable marker or a gene product conferring insecticidal resistance, herbicide tolerance, small RNA expression, nitrogen use efficiency, water use efficiency, or nutritional quality.

### 1. Insect Resistance

Various insect resistance genes can be expressed by a chimeric regulatory element comprising SEQ ID NO:9, SEQ ID NOs:25-583, SEQ ID NOs:585-2268. The chimeric regulatory element comprising SEQ ID NO:9, SEQ ID NOs:25-583, SEQ ID NOs:585-2268 can be operably linked with at least one other gene expression cassette containing an insect resistance gene. The operably linked sequences can then be incorporated into a chosen vector to allow for identification and selection of transformed plants (“transformants”). Exemplary insect resistance coding sequences are known in the art. As embodiments of insect resistance coding sequences that can be operably linked to the regulatory elements of the subject disclosure, the following traits are provided. Coding sequences that provide exemplary Lepidopteran insect resistance include: cry1A; cry1A.105; cry1Ab; cry1Ab (truncated); cry1Ab-Ac (fusion protein); cry1Ac (marketed as Widestrike®); cry1C; cry1F (marketed as Widestrike®); cry1Fa2; cry2Ab2; cry2Ae; cry9C; mocry1F; pinII (protease inhibitor protein); vip3A(a); and vip3Aa20. Coding sequences that provide exemplary Coleopteran insect resistance include: cry34Ab1 (marketed as Herculex®); cry35Ab1 (marketed as Herculex®); cry3A; cry3Bb1; dvsnf7; and mcry3A. Coding sequences that provide exemplary multi-insect resistance include ecry31.Ab. The above list of insect resistance genes is not meant to be limiting. Any insect resistance genes are encompassed by the present disclosure.

### 2. Herbicide Tolerance

Various herbicide tolerance genes be can be expressed by a chimeric regulatory element comprising SEQ ID NO:9, SEQ ID NOs:25-583, SEQ ID NOs:585-2268. The chimeric regulatory element comprising SEQ ID NO:9, SEQ ID NOs:25-583, SEQ ID NOs:585-2268 can be operably linked with at least one other gene expression cassette containing a herbicide tolerance gene. The operably linked sequences can then be incorporated into a chosen vector to allow for identification and selection of transformed plants (“transformants”). Exemplary herbicide tolerance coding sequences are known in the art. As embodiments of herbicide tolerance coding sequences that can be operably linked to the regulatory elements of the subject disclosure, the following traits are provided. The glyphosate herbicide contains a mode of action by inhibiting the EPSPS enzyme (5-enolpyruvylshikimate-3-phosphate synthase). This enzyme is involved in the biosynthesis of aromatic amino acids that are essential for growth and development of plants. Various enzymatic mechanisms are known in the art that can be utilized to inhibit this enzyme. The genes that encode such enzymes can be operably linked to the gene regulatory elements of the subject disclosure. In an embodiment, selectable marker genes include, but are not limited to genes encoding glyphosate resistance genes include: mutant EPSPS genes such as 2mEPSPS genes, cp4 EPSPS genes, mEPSPS genes, dgt-28 genes; aroA genes; and glyphosate degradation genes such as glyphosate acetyl transferase genes (gat) and glyphosate oxidase genes (gox). These traits are currently marketed as Gly-Tol™, Optimum® GAT®, Agrisure® GT and Roundup Ready®. Resistance genes for glufosinate and/or bialaphos compounds include dsm-2, bar and pat genes. The bar and pat traits are currently marketed as LibertyLink®. Also included are tolerance genes that provide resistance to 2,4-D such as aad-1 genes (it should be noted that aad-1 genes have further activity on arloxyphenoxypropionate herbicides) and aad-12 genes (it should be noted that aad-12 genes have further activity on pyidyloxyacetate synthetic auxins). These traits are marketed as Enlist® crop protection technology. Resistance genes for ALS inhibitors (sulfonylureas, imidazolinones, triazolopyrimidines, pyrimidinylthiobenzoates, and sulfonylamino-carbonyl-triazolinones) are known in the art. These resistance genes most commonly result from point mutations to the ALS encoding gene sequence. Other ALS inhibitor resistance genes include hra genes, the csr1-2 genes, Sr-HrA genes, and surB genes. Some of the traits are marketed under the tradename Clearfield®. Herbicides that inhibit HPPD include the pyrazolones such as pyrazoxyfen, benzofenap, and topramezone; triketones such as mesotrione, sulcotrione, tembotrione, benzobicyclon; and diketonitriles such as isoxaflutole. These exemplary HPPD herbicides can be tolerated by known traits. Examples of HPPD inhibitors include hppdPF_W336 genes (for resistance to isoxaflutole) and avhppd-03 genes (for resistance to meostrione). An example of oxynil herbicide tolerant traits include the bxn gene, which has been showed to impart resistance to the herbicide/antibiotic bromoxynil. Resistance genes for dicamba include the dicamba monooxygenase gene (dmo) as disclosed in International PCT Publication No. WO 2008/105890. Resistance genes for PPO or PROTOX inhibitor type herbicides (e.g., acifluorfen, butafenacil, flupropazil, pentoxazone, carfentrazone, fluazolate, pyraflufen, aclonifen, azafenidin, flumioxazin, flumiclorac, bifenox, oxyfluorfen, lactofen, fomesafen, fluoroglycofen, and sulfentrazone) are known in the art. Exemplary genes conferring resistance to PPO include over expression of a wild-type Arabidopsis thaliana PPO enzyme (Lermontova I and Grimm B, (2000) Overexpression of plastidic protoporphyrinogen IX oxidase leads to resistance to the diphenyl-ether herbicide acifluorfen. Plant Physiol 122:75-83.), the B. subtilis PPO gene (Li, X. and Nicholl D. 2005. Development of PPO inhibitor-resistant cultures and crops. Pest Manag. Sci. 61:277-285 and Choi K W, Han O, Lee H J, Yun Y C, Moon Y H, Kim M K, Kuk Y I, Han S U and Guh J O, (1998) Generation of resistance to the diphenyl ether herbicide, oxyfluorfen, via expression of the Bacillus subtilis protoporphyrinogen oxidase gene in transgenic tobacco plants. Biosci Biotechnol Biochem 62:558-560.) Resistance genes for pyridinoxy or phenoxy proprionic acids and cyclohexones include the ACCase inhibitor-encoding genes (e.g., Acc1-S1, Acc1-S2 and Acc1-S3). Exemplary genes conferring resistance to cyclohexanediones and/or aryloxyphenoxypropanoic acid include haloxyfop, diclofop, fenoxyprop, fluazifop, and quizalofop. Finally, herbicides can inhibit photosynthesis, including triazine or benzonitrile are provided tolerance by psbA genes (tolerance to triazine), 1+ genes (tolerance to triazine), and nitrilase genes (tolerance to benzonitrile). The above list of herbicide tolerance genes is not meant to be limiting. Any herbicide tolerance genes are encompassed by the present disclosure.

### 3. Agronomic Traits

Various agronomic trait genes can be can be expressed by a chimeric regulatory element comprising SEQ ID NO:9, SEQ ID NOs:25-583, SEQ ID NOs:585-2268. The chimeric regulatory element comprising SEQ ID NO:9, SEQ ID NOs:25-583, SEQ ID NOs:585-2268 can be operably linked with at least one other gene expression cassette containing an agronomic trait gene. The operably linked sequences can then be incorporated into a chosen vector to allow for identification and selection of transformed plants (“transformants”). Exemplary agronomic trait coding sequences are known in the art. As embodiments of agronomic trait coding sequences that can be operably linked to the regulatory elements of the subject disclosure, the following traits are provided. Delayed fruit softening as provided by the pg genes inhibit the production of polygalacturonase enzyme responsible for the breakdown of pectin molecules in the cell wall, and thus causes delayed softening of the fruit. Further, delayed fruit ripening/senescence of acc genes act to suppress the normal expression of the native acc synthase gene, resulting in reduced ethylene production and delayed fruit ripening. Whereas, the accd genes metabolize the precursor of the fruit ripening hormone ethylene, resulting in delayed fruit ripening. Alternatively, the sam-k genes cause delayed ripening by reducing S-adenosylmethionine (SAM), a substrate for ethylene production. Drought stress tolerance phenotypes as provided by cspB genes maintain normal cellular functions under water stress conditions by preserving RNA stability and translation. Another example includes the EcBetA genes that catalyze the production of the osmoprotectant compound glycine betaine conferring tolerance to water stress. In addition, the RmBetA genes catalyze the production of the osmoprotectant compound glycine betaine conferring tolerance to water stress. Photosynthesis and yield enhancement is provided with the bbx32 gene that expresses a protein that interacts with one or more endogenous transcription factors to regulate the plant's day/night physiological processes. Ethanol production can be increase by expression of the amy797E genes that encode a thermostable alpha-amylase enzyme that enhances bioethanol production by increasing the thermostability of amylase used in degrading starch. Finally, modified amino acid compositions can result by the expression of the cordapA genes that encode a dihydrodipicolinate synthase enzyme that increases the production of amino acid lysine. The above list of agronomic trait coding sequences is not meant to be limiting. Any agronomic trait coding sequence is encompassed by the present disclosure.

### 4. DNA Binding Proteins

Various DNA binding transgene/heterologous coding sequences can be can be expressed by a chimeric regulatory element comprising SEQ ID NO:9, SEQ ID NOs:25-583, SEQ ID NOs:585-2268. The chimeric regulatory element comprising SEQ ID NO:9, SEQ ID NOs:25-583, SEQ ID NOs:585-2268 can be operably linked with at least one other gene expression cassette containing a DNA binding gene. The operably linked sequences can then be incorporated into a chosen vector to allow for identification and selectable of transformed plants (“transformants”). Exemplary DNA binding protein coding sequences are known in the art. As embodiments of DNA binding protein coding sequences that can be operably linked to the regulatory elements of the subject disclosure, the following types of DNA binding proteins can include; Zinc Fingers, TALENS, CRISPRS, and meganucleases. The above list of DNA binding protein coding sequences is not meant to be limiting. Any DNA binding protein coding sequences is encompassed by the present disclosure.

### 5. Small RNA

Various small RNA sequences can be can be expressed by a chimeric regulatory element comprising SEQ ID NO:9, SEQ ID NOs:25-583, SEQ ID NOs:585-2268. The chimeric regulatory element comprising SEQ ID NO:9, SEQ ID NOs:25-583, SEQ ID NOs:585-2268 can be operably linked with at least one other gene expression cassette containing a small RNA sequence. The operably linked sequences can then be incorporated into a chosen vector to allow for identification and selection of transformed plants (“transformants”). Exemplary small RNA traits are known in the art. As embodiments of small RNA coding sequences that can be operably linked to the regulatory elements of the subject disclosure, the following traits are provided. For example, delayed fruit ripening/senescence of the anti-efe small RNA delays ripening by suppressing the production of ethylene via silencing of the ACO gene that encodes an ethylene-forming enzyme. The altered lignin production of ccomt small RNA reduces content of guanacyl (G) lignin by inhibition of the endogenous S-adenosyl-L-methionine: trans-caffeoyl CoA 3-O-methyltransferase (CCOMT gene). Further, the Black Spot Bruise Tolerance in Solanum verrucosum can be reduced by the Ppo5 small RNA which triggers the degradation of Ppo5 transcripts to block black spot bruise development. Also included is the dvsnf7 small RNA that inhibits Western Corn Rootworm with dsRNA containing a 240 bp fragment of the Western Corn Rootworm Snf7 gene. Modified starch/carbohydrates can result from small RNA such as the pPhL small RNA (degrades PhL transcripts to limit the formation of reducing sugars through starch degradation) and pR1 small RNA (degrades R1 transcripts to limit the formation of reducing sugars through starch degradation). Additional, benefits such as reduced acrylamide resulting from the asn1 small RNA that triggers degradation of Asn1 to impair asparagine formation and reduce polyacrylamide. Finally, the non-browning phenotype of pgas ppo suppression small RNA results in suppressing PPO to produce apples with a non-browning phenotype. The above list of small RNAs is not meant to be limiting. Any small RNA encoding sequences are encompassed by the present disclosure.

### 6. Selectable Markers

Various selectable markers also described as reporter genes can be can be expressed by a chimeric regulatory element comprising SEQ ID NO:9, SEQ ID NOs:25-583, SEQ ID NOs:585-2268. The chimeric regulatory element comprising SEQ ID NO:9, SEQ ID NOs:25-583, SEQ ID NOs:585-2268 can be operably linked with at least one other gene expression cassette containing a reporter gene. The operably linked sequences can then be incorporated into a chosen vector to allow for identification and selectable of transformed plants (“transformants”). Many methods are available to confirm expression of selectable markers in transformed plants, including for example DNA sequencing and PCR (polymerase chain reaction), Southern blotting, RNA blotting, immunological methods for detection of a protein expressed from the vector. But, usually the reporter genes are observed through visual observation of proteins that when expressed produce a colored product. Exemplary reporter genes are known in the art and encode β-glucuronidase (GUS), luciferase, green fluorescent protein (GFP), yellow fluorescent protein (YFP, Phi-YFP), red fluorescent protein (DsRFP, RFP, etc), β-galactosidase, and the like (See Sambrook, et al., Molecular Cloning: A Laboratory Manual, Third Edition, Cold Spring Harbor Press, N.Y., 2001, the content of which is incorporated herein by reference in its entirety).

Selectable marker genes are utilized for selection of transformed cells or tissues. Selectable marker genes include genes encoding antibiotic resistance, such as those encoding neomycin phosphotransferase II (NEO), spectinomycin/streptinomycin resistance (AAD), and hygromycin phosphotransferase (HPT or HGR) as well as genes conferring resistance to herbicidal compounds. Herbicide resistance genes generally code for a modified target protein insensitive to the herbicide or for an enzyme that degrades or detoxifies the herbicide in the plant before it can act. For example, resistance to glyphosate has been obtained by using genes coding for mutant target enzymes, 5-enolpyruvylshikimate-3-phosphate synthase (EPSPS). Genes and mutants for EPSPS are well known, and further described below. Resistance to glufosinate ammonium, bromoxynil, and 2,4-dichlorophenoxyacetate (2,4-D) have been obtained by using bacterial genes encoding PAT or DSM-2, a nitrilase, an AAD-1, or an AAD-12, each of which are examples of proteins that detoxify their respective herbicides.

In an embodiment, herbicides can inhibit the growing point or meristem, including imidazolinone or sulfonylurea, and genes for resistance/tolerance of acetohydroxyacid synthase (AHAS) and acetolactate synthase (ALS) for these herbicides are well known. Glyphosate resistance genes include mutant 5-enolpyruvylshikimate-3-phosphate synthase (EPSPs) and dgt-28 genes (via the introduction of recombinant nucleic acids and/or various forms of in vivo mutagenesis of native EPSPs genes), aroA genes and glyphosate acetyl transferase (GAT) genes, respectively). Resistance genes for other phosphono compounds include bar and pat genes from Streptomyces species, including Streptomyces hygroscopicus and Streptomyces viridichromogenes, and pyridinoxy or phenoxy proprionic acids and cyclohexones (ACCase inhibitor-encoding genes). Exemplary genes conferring resistance to cyclohexanediones and/or aryloxyphenoxypropanoic acid (including haloxyfop, diclofop, fenoxyprop, fluazifop, quizalofop) include genes of acetyl coenzyme A carboxylase (ACCase); Acc1-S1, Acc1-S2 and Acc1-S3. In an embodiment, herbicides can inhibit photosynthesis, including triazine (psbA and Is+genes) or benzonitrile (nitrilase gene). Futhermore, such selectable markers can include positive selection markers such as phosphomannose isomerase (PMI) enzyme.

In an embodiment, selectable marker genes include, but are not limited to genes encoding: 2,4-D; neomycin phosphotransferase II; cyanamide hydratase; aspartate kinase; dihydrodipicolinate synthase; tryptophan decarboxylase; dihydrodipicolinate synthase and desensitized aspartate kinase; bar gene; tryptophan decarboxylase; neomycin phosphotransferase (NEO); hygromycin phosphotransferase (HPT or HYG); dihydrofolate reductase (DHFR); phosphinothricin acetyltransferase; 2,2-dichloropropionic acid dehalogenase; acetohydroxyacid synthase; 5-enolpyruvyl-shikimate-phosphate synthase (aroA); haloarylnitrilase; acetyl-coenzyme A carboxylase; dihydropteroate synthase (sul I); and 32 kD photosystem II polypeptide (psbA). An embodiment also includes selectable marker genes encoding resistance to: chloramphenicol; methotrexate; hygromycin; spectinomycin; bromoxynil; glyphosate; and phosphinothricin. The above list of selectable marker genes is not meant to be limiting. Any reporter or selectable marker gene are encompassed by the present disclosure.

In some embodiments the coding sequences are synthesized for optimal expression in a plant. For example, in an embodiment, a coding sequence of a gene has been modified by codon optimization to enhance expression in plants. An insecticidal resistance transgene, an herbicide tolerance transgene, a nitrogen use efficiency transgene, a water use efficiency transgene, a nutritional quality transgene, a DNA binding transgene, or a selectable marker transgene/heterologous coding sequence can be optimized for expression in a particular plant species or alternatively can be modified for optimal expression in dicotyledonous or monocotyledonous plants. Plant preferred codons may be determined from the codons of highest frequency in the proteins expressed in the largest amount in the particular plant species of interest. In an embodiment, a coding sequence, gene, heterologous coding sequence or transgene/heterologous coding sequence is designed to be expressed in plants at a higher level resulting in higher transformation efficiency. Methods for plant optimization of genes are well known. Guidance regarding the optimization and production of synthetic DNA sequences can be found in, for example, WO2013016546, WO2011146524, WO1997013402, U.S. Pat. Nos. 6,166,302, and 5,380,831, herein incorporated by reference.

### Molecular Confirmation

Methods of confirming the presence of SEQ ID NO:9, SEQ ID NOs:25-583, SEQ ID NOs:585-2268 within the genome of a plant are known in the art. For example the detection of SEQ ID NO:9, SEQ ID NOs:25-583, SEQ ID NOs:585-2268 within the genome of a plant can be achieved, for example, by the polymerase chain reaction (PCR). The PCR detection is done by the use of two oligonucleotide primers flanking the polymorphic segment of the polymorphism followed by DNA amplification. This step involves repeated cycles of heat denaturation of the DNA followed by annealing of the primers to their complementary sequences at low temperatures, and extension of the annealed primers with DNA polymerase. Size separation of DNA fragments on agarose or polyacrylamide gels following amplification, comprises the major part of the methodology. Such selection and screening methodologies are well known to those skilled in the art. Molecular confirmation methods that can be used to identify transgenic plants are known to those with skill in the art. Several exemplary methods are further described below.

Molecular Beacons have been described for use in sequence detection. Briefly, a FRET oligonucleotide probe is designed that overlaps the flanking genomic and insert DNA junction. The unique structure of the FRET probe results in it containing a secondary structure that keeps the fluorescent and quenching moieties in close proximity. The FRET probe and PCR primers (one primer in the insert DNA sequence and one in the flanking genomic sequence) are cycled in the presence of a thermostable polymerase and dNTPs. Following successful PCR amplification, hybridization of the FRET probe(s) to the target sequence results in the removal of the probe secondary structure and spatial separation of the fluorescent and quenching moieties. A fluorescent signal indicates the presence of the flanking genomic/transgene insert sequence due to successful amplification and hybridization. Such a molecular beacon assay for detection of as an amplification reaction is an embodiment of the subject disclosure.

Hydrolysis probe assay, otherwise known as TAQMAN® (Life Technologies, Foster City, Calif.), is a method of detecting and quantifying the presence of a DNA sequence. Briefly, a FRET oligonucleotide probe is designed with one oligo within the transgene and one in the flanking genomic sequence for event-specific detection. The FRET probe and PCR primers (one primer in the insert DNA sequence and one in the flanking genomic sequence) are cycled in the presence of a thermostable polymerase and dNTPs. Hybridization of the FRET probe results in cleavage and release of the fluorescent moiety away from the quenching moiety on the FRET probe. A fluorescent signal indicates the presence of the flanking/transgene insert sequence due to successful amplification and hybridization. Such a hydrolysis probe assay for detection of as an amplification reaction is an embodiment of the subject disclosure.

KASPar® assays are a method of detecting and quantifying the presence of a DNA sequence. Briefly, the genomic DNA sample comprising the integrated gene expression cassette polynucleotide is screened using a polymerase chain reaction (PCR) based assay known as a KASPar® assay system. The KASPar® assay used in the practice of the subject disclosure can utilize a KASPar® PCR assay mixture which contains multiple primers. The primers used in the PCR assay mixture can comprise at least one forward primers and at least one reverse primer. The forward primer contains a sequence corresponding to a specific region of the DNA polynucleotide, and the reverse primer contains a sequence corresponding to a specific region of the genomic sequence. In addition, the primers used in the PCR assay mixture can comprise at least one forward primers and at least one reverse primer. For example, the KASPar® PCR assay mixture can use two forward primers corresponding to two different alleles and one reverse primer. One of the forward primers contains a sequence corresponding to specific region of the endogenous genomic sequence. The second forward primer contains a sequence corresponding to a specific region of the DNA polynucleotide. The reverse primer contains a sequence corresponding to a specific region of the genomic sequence. Such a KASPar® assay for detection of an amplification reaction is an embodiment of the subject disclosure.

In some embodiments the fluorescent signal or fluorescent dye is selected from the group consisting of a HEX fluorescent dye, a FAM fluorescent dye, a JOE fluorescent dye, a TET fluorescent dye, a Cy 3 fluorescent dye, a Cy 3.5 fluorescent dye, a Cy 5 fluorescent dye, a Cy 5.5 fluorescent dye, a Cy 7 fluorescent dye, and a ROX fluorescent dye.

In other embodiments the amplification reaction is run using suitable second fluorescent DNA dyes that are capable of staining cellular DNA at a concentration range detectable by flow cytometry, and have a fluorescent emission spectrum which is detectable by a real time thermocycler. It should be appreciated by those of ordinary skill in the art that other nucleic acid dyes are known and are continually being identified. Any suitable nucleic acid dye with appropriate excitation and emission spectra can be employed, such as YO-PRO-1®, SYTOX Green®, SYBR Green I®, SYTO11®, SYTO12®, SYTO13®, BOBO®, YOYO®, and TOTO®.

In further embodiments, Next Generation Sequencing (NGS) can be used for detection. As described by Brautigma et al., 2010, DNA sequence analysis can be used to determine the nucleotide sequence of the isolated and amplified fragment. The amplified fragments can be isolated and sub-cloned into a vector and sequenced using chain-terminator method (also referred to as Sanger sequencing) or Dye-terminator sequencing. In addition, the amplicon can be sequenced with Next Generation Sequencing. NGS technologies do not require the sub-cloning step, and multiple sequencing reads can be completed in a single reaction. Three NGS platforms are commercially available, the Genome Sequencer FLX™ from 454 Life Sciences/Roche, the Illumina Genome Analyser™ from Solexa and Applied Biosystems' SOLiD™ (acronym for: ‘Sequencing by Oligo Ligation and Detection’). In addition, there are two single molecule sequencing methods that are currently being developed. These include the true Single Molecule Sequencing (tSMS) from Helicos Bioscience™ and the Single Molecule Real Time™ sequencing (SMRT) from Pacific Biosciences.

The Genome Sequencher FLX™ which is marketed by 454 Life Sciences/Roche is a long read NGS, which uses emulsion PCR and pyrosequencing to generate sequencing reads. DNA fragments of 300-800 bp or libraries containing fragments of 3-20 kb can be used. The reactions can produce over a million reads of about 250 to 400 bases per run for a total yield of 250 to 400 megabases. This technology produces the longest reads but the total sequence output per run is low compared to other NGS technologies.

The Illumina Genome Analyser™ which is marketed by Solexa™ is a short read NGS which uses sequencing by synthesis approach with fluorescent dye-labeled reversible terminator nucleotides and is based on solid-phase bridge PCR. Construction of paired end sequencing libraries containing DNA fragments of up to 10 kb can be used. The reactions produce over 100 million short reads that are 35-76 bases in length. This data can produce from 3-6 gigabases per run.

The Sequencing by Oligo Ligation and Detection (SOLiD) system marketed by Applied Biosystems™ is a short read technology. This NGS technology uses fragmented double stranded DNA that are up to 10 kb in length. The system uses sequencing by ligation of dye-labelled oligonucleotide primers and emulsion PCR to generate one billion short reads that result in a total sequence output of up to 30 gigabases per run.

The tSMS of Helicos Bioscience™ and SMRT of Pacific Biosciences™ apply a different approach which uses single DNA molecules for the sequence reactions. The tSMS Helicos™ system produces up to 800 million short reads that result in 21 gigabases per run. These reactions are completed using fluorescent dye-labelled virtual terminator nucleotides that is described as a ‘sequencing by synthesis’ approach.

The SMRT Next Generation Sequencing system marketed by Pacific Biosciences™ uses a real time sequencing by synthesis. This technology can produce reads of up to 1,000 bp in length as a result of not being limited by reversible terminators. Raw read throughput that is equivalent to one-fold coverage of a diploid human genome can be produced per day using this technology.

### Transgenic Plants

In an embodiment, a plant, plant tissue, or plant cell comprises a chimeric regulatory element comprising SEQ ID NO:9, SEQ ID NOs:25-583, SEQ ID NOs:585-2268. In one embodiment a plant, plant tissue, or plant cell comprises the cis-acting regulatory element of a sequence selected from SEQ ID NO:9, SEQ ID NOs:25-583, SEQ ID NOs:585-2268 or a sequence that has 80%, 85%, 90%, 95% or 99.5% sequence identity with a sequence selected from SEQ ID NO:9, SEQ ID NOs:25-583, SEQ ID NOs:585-2268. In an embodiment, a plant, plant tissue, or plant cell comprises a gene expression cassette comprising a sequence selected from SEQ ID NO:9, SEQ ID NOs:25-583, SEQ ID NOs:585-2268, or a sequence that has 80%, 85%, 90%, 95% or 99.5% sequence identity with a sequence selected from SEQ ID NO:9, SEQ ID NOs:25-583, SEQ ID NOs:585-2268 that is operably linked to a heterologous coding sequence. In an illustrative embodiment, a plant, plant tissue, or plant cell comprises a gene expression cassette comprising a chimeric regulatory element comprising SEQ ID NO:9, SEQ ID NOs:25-583, SEQ ID NOs:585-2268, or a sequence that has 80%, 85%, 90%, 95% or 99.5% sequence identity with a sequence selected from SEQ ID NO:9, SEQ ID NOs:25-583, SEQ ID NOs:585-2268, wherein the chimeric regulatory element is operably linked to a transgene or heterologous coding sequence is an insecticidal resistance transgene, an herbicide tolerance transgene, a nitrogen use efficiency transgene, a water use efficiency transgene, a nutritional quality transgene, a DNA binding transgene, a selectable marker transgene, or combinations thereof.

In accordance with one embodiment a plant, plant tissue, or plant cell is provided wherein the plant, plant tissue, or plant cell comprises a cis-acting regulatory element comprising SEQ ID NO:9, SEQ ID NOs:25-583, SEQ ID NOs:585-2268 operably linked to a transgene, wherein cis-acting regulatory element comprising SEQ ID NO:9, SEQ ID NOs:25-583, SEQ ID NOs:585-2268 derived sequence comprises a sequence having 80%, 85%, 90%, 95% or 99.5% sequence identity with SEQ ID NO:9, SEQ ID NOs:25-583, SEQ ID NOs:585-2268. In one embodiment a plant, plant tissue, or plant cell is provided wherein the plant, plant tissue, or plant cell comprises a cis-acting regulatory element comprising SEQ ID NO:9, SEQ ID NOs:25-583, SEQ ID NOs:585-2268, or a sequence that has 80%, 85%, 90%, 95% or 99.5% sequence identity with SEQ ID NO:9, SEQ ID NOs:25-583, SEQ ID NOs:585-2268 operably linked to a heterologous coding sequence. In accordance with one embodiment the plant, plant tissue, or plant cell comprises a chimeric regulatory molecule comprising SEQ ID NO:9, SEQ ID NOs:25-583, SEQ ID NOs:585-2268 or a sequence having 80%, 85%, 90%, 95% or 99.5% sequence identity with SEQ ID NO:9, SEQ ID NOs:25-583, SEQ ID NOs:585-2268 operably linked to a heterologous coding sequence. In one embodiment the plant, plant tissue, or plant cell comprises a chimeric regulatory molecule operably linked to a transgene/heterologous coding sequence wherein the chimeric regulatory molecule consists of SEQ ID NO:2 or a sequence having 80%, 85%, 90%, 95% or 99.5% sequence identity with SEQ ID NO:9, SEQ ID NOs:25-583, SEQ ID NOs:585-2268. In accordance with one embodiment the gene construct comprising a chimeric regulatory molecule comprising SEQ ID NO:9, SEQ ID NOs:25-583, SEQ ID NOs:585-2268 operably linked to a transgene/heterologous coding sequence is incorporated into the genome of the plant, plant tissue, or plant cell.

In one embodiment the plant, plant tissue, or plant cell is a dicotyledonous or monocotyledonous plant or a cell or tissue derived from a dicotyledonous or monocotyledonous plant. In one embodiment the plant is selected from the group consisting of wheat, rice, sorghum, oats, rye, bananas, sugar cane, soybean, cotton, sunflower, Zea mays, alfalfa, rapeseed, canola, Indian mustard, Ethiopian mustard, beans, broccoli, cabbage, cauliflower, celery, cucumber, eggplant, lettuce; melon, pea, pepper, peanut, potato, pumpkin, radish, spinach, sugarbeet, tobacco, tomato, and watermelon.

One of skill in the art will recognize that after the exogenous sequence is stably incorporated in transgenic plants and confirmed to be operable, it can be introduced into other plants by sexual crossing. Any of a number of standard breeding techniques can be used, depending upon the species to be crossed.

The present disclosure also encompasses seeds of the transgenic plants described above, wherein the seed has the transgene/heterologous coding sequence or gene construct containing the gene regulatory elements of the subject disclosure. The present disclosure further encompasses the progeny, clones, cell lines or cells of the transgenic plants described above wherein said progeny, clone, cell line or cell has the transgene/heterologous coding sequence or gene construct containing the gene regulatory elements of the subject disclosure.

The present disclosure also encompasses the cultivation of transgenic plants described above, wherein the transgenic plant has the transgene/heterologous coding sequence or gene construct containing the gene regulatory elements of the subject disclosure. Accordingly, such transgenic plants may be engineered to, inter alia, have one or more desired traits or transgenic events containing the gene regulatory elements of the subject disclosure, by being transformed with nucleic acid molecules according to the invention, and may be cropped or cultivated by any method known to those of skill in the art.

### Method of Expressing a Transgene

In an embodiment, a method of expressing at least one transgene/heterologous coding sequence in a plant comprises growing a plant comprising a chimeric regulatory molecule comprising SEQ ID NO:9, SEQ ID NOs:25-583, SEQ ID NOs:585-2268 operably linked to at least one transgene/heterologous coding sequence or a polylinker sequence. In an embodiment the cis-acting regulatory element or a chimeric regulatory element consists of a sequence selected from SEQ ID NO:9, SEQ ID NOs:25-583, SEQ ID NOs:585-2268 or a sequence that has 80%, 85%, 90%, 95% or 99.5% sequence identity with a sequence selected from SEQ ID NO:9, SEQ ID NOs:25-583, SEQ ID NOs:585-2268. In an embodiment, a method of expressing at least one transgene/heterologous coding sequence in a plant comprising growing a plant comprising a chimeric regulatory molecule comprising SEQ ID NO:9, SEQ ID NOs:25-583, SEQ ID NOs:585-2268 operably linked to at least one transgene. In an embodiment, a method of expressing at least one transgene/heterologous coding sequence in a plant tissue or plant cell comprising culturing a plant tissue or plant cell comprising a chimeric regulatory molecule comprising SEQ ID NO:9, SEQ ID NOs:25-583, SEQ ID NOs:585-2268 operably linked to at least one transgene.

In an embodiment, a method of expressing at least one transgene/heterologous coding sequence in a plant comprises growing a plant comprising a gene expression cassette comprising a chimeric regulatory molecule comprising SEQ ID NO:9, SEQ ID NOs:25-583, SEQ ID NOs:585-2268 operably linked to at least one transgene. In one embodiment the cis acting regulatory element or a chimeric regulatory element consists of a sequence selected from SEQ ID NO:9, SEQ ID NOs:25-583, SEQ ID NOs:585-2268 or a sequence that has 80%, 85%, 90%, 95% or 99.5% sequence identity with a sequence selected from SEQ ID NO:9, SEQ ID NOs:25-583, SEQ ID NOs:585-2268. In an embodiment, a method of expressing at least one transgene/heterologous coding sequence in a plant comprises growing a plant comprising a gene expression cassette comprising a chimeric regulatory molecule comprising SEQ ID NO:9, SEQ ID NOs:25-583, SEQ ID NOs:585-2268 operably linked to at least one transgene. In an embodiment, a method of expressing at least one transgene/heterologous coding sequence in a plant comprises growing a plant or a gene expression cassette comprising a chimeric regulatory molecule comprising SEQ ID NO:9, SEQ ID NOs:25-583, SEQ ID NOs:585-2268 operably linked to at least one transgene. In an embodiment, a method of expressing at least one transgene/heterologous coding sequence in a plant tissue or plant cell comprises culturing a plant tissue or plant cell comprising a gene expression cassette containing a chimeric regulatory molecule comprising SEQ ID NO:9, SEQ ID NOs:25-583, SEQ ID NOs:585-2268 operably linked to at least one transgene. In an embodiment, a method of expressing at least one transgene/heterologous coding sequence in a plant tissue or plant cell comprises culturing a plant tissue or plant cell comprising a gene expression cassette, a chimeric regulatory molecule comprising SEQ ID NO:9, SEQ ID NOs:25-583, SEQ ID NOs:585-2268 operably linked to at least one transgene.

All references, including publications, patents, and patent applications, cited herein are hereby incorporated by reference to the extent they are not inconsistent with the explicit details of this disclosure, and are so incorporated to the same extent as if each reference were individually and specifically indicated to be incorporated by reference and were set forth in its entirety herein. The references discussed herein are provided solely for their disclosure prior to the filing date of the present application. Nothing herein is to be construed as an admission that the inventors are not entitled to antedate such disclosure by virtue of prior invention.

Embodiments of the subject disclosure are further exemplified in the following Examples. It should be understood that these Examples are given by way of illustration only. From the above embodiments and the following Examples, one skilled in the art can ascertain the essential characteristics of this disclosure, and without departing from the spirit and scope thereof, can make various changes and modifications of the embodiments of the disclosure to adapt it to various usages and conditions. Thus, various modifications of the embodiments of the disclosure, in addition to those shown and described herein, will be apparent to those skilled in the art from the foregoing description. Such modifications are also intended to fall within the scope of the appended claims. The following is provided by way of illustration and not intended to limit the scope of the invention.

### EXAMPLES

### Example 1: Identification of Cis-Acting Regulatory Elements

Cis-acting regulatory elements were obtained from the PLACE database (Higo, K., Ugawa, Y., Iwamoto, M. & Korenaga, T. 1999. “Plant cis-acting regulatory DNA elements (PLACE) database: 1999” NAR. 27:297-300.). The elements were sorted by size and a subset of ten elements were selected for testing based on the following criteria: 1) be between 18 and 24 nucleotides in length (inclusive), 2) not be overly simple in sequence, e.g., ‘TTTTTAAAAA’ and 3) not contain ambiguous nucleotides. The cis-acting regulatory elements that were obtained are listed as SEQ ID NO:1-10. The selected elements and their details are shown in Table 1.

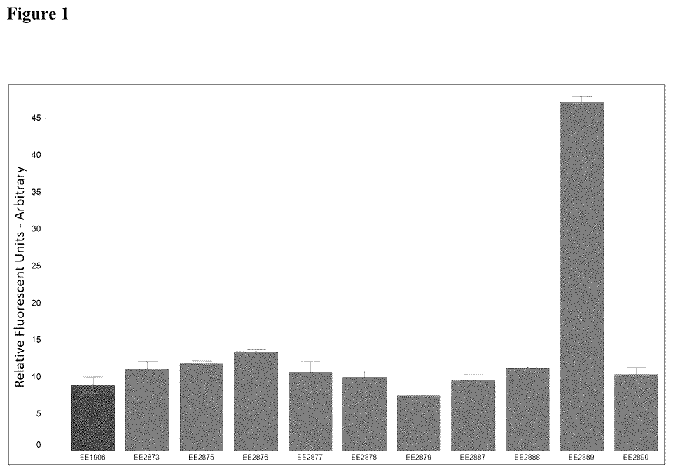

TABLE 1
Elements selected for testing from the PLACE database.

| | | | | Cis-acting | | | |
|---|---|---|---|---|---|---|---|
| | | | | element | | | |
| | | | | cloned as a | | | |
| | Database | | SEQ | modulating | | | |
| | Entry | | ID | element of | FIG. 1 | RFU | RFU |
| Element Name | Number | Sequence | NO: | a promoter | Label: | Mean | SEM |
| CARGIATAP3 | S000347 | GTTTACATAAATGGAAAA | SEQ ID | SEQ ID | EE2873 | 11.15 | 1.00 |
| | | | NO: 1 | NO: 13 | | | |
| GLUTEBOX1OSGT3 | S000128 | TATCTAGTGAGTCACTTCA | SEQ ID | SEQ ID | EE2875 | 11.88 | 0.33 |
| | | | NO: 2 | NO: 14 | | | |
| ANAEROBICCISZMGAPC4 | S000350 | CGAAACCAGCAACGGTCCAG | SEQ ID | SEQ ID | EE2876 | 13.42 | 0.4 |
| | | | NO: 3 | NO: 15 | | | |
| GAREHVAMY1 | S000038 | GGCCGATAACAAACTCCGGCC | SEQ ID | SEQ ID | EE2877 | 10.65 | 1.52 |
| | | | NO: 4 | NO: 16 | | | |
| PE1ASPHYA3 | S000196 | GAAATAGCAAATGTTAAAAATA | SEQ ID | SEQ ID | EE2878 | 10.01 | 0.84 |
| | | | NO: 5 | NO: 17 | | | |
| 23BPUASNSCYCB1 | S000283 | TTTATTTACCAAACGGTAACATC | SEQ ID | SEQ ID | EE2879 | 7.53 | 0.45 |
| | | | NO: 6 | NO: 18 | | | |
| ELEMENT1GMLBC3 | S000319 | GATATATTAATATTTTATTTTATA | SEQ ID | SEQ ID | EE2887 | 9.63 | 0.7 |
| | | | NO: 7 | NO: 19 | | | |
| GREGIONNTPRB1B | S000364 | TGGCGGCTCTTATCTCACGTGATG | SEQ ID | SEQ ID | EE2888 | 11.26 | 0.23 |
| | | | NO: 8 | NO: 20 | | | |
| JERECRSTR | S000384 | CTCTTAGACCGCCTTCTTTGAAAG | SEQ ID | SEQ ID | EE2889 | 47.14 | 0.86 |
| | | | NO: 9 | NO: 21 | | | |
| SE1PVGRP18 | S000288 | ATAATGGGCCACACTGTGGGGCAT | SEQ ID | SEQ ID | EE2890 | 10.36 | 0.96 |
| | | | NO: 10 | NO: 22 | | | |

### Example 2: Synthesis of the Cis-Acting Regulatory Element as a Modulating Element Within a Regulatory Element (for Example a Promoter Regulatory Element)

The selected elements were engineered as a modulating element within a regulatory element (for example a promoter regulatory element). The regulatory elements were fused to a promoter as a multimer of cassettes with the intervening sequences, ‘SEQ ID NO:11; CATAAACACC’ and ‘SEQ ID NO:12; GGGCACGCGTC’ as spacers in the 5′ and 3′ positions, respectively. In this example three cassettes were synthesized into the promoter. These three cassettes were synthesized along with and upstream of the CaMV 35S minimal promoter (SEQ ID NO:13) and the TMV omega 5′ UTR (SEQ ID NO:14) by GenScript Biotech (Piscataway, NJ). The restriction endonuclease sites EcoRI and NcoI were included on the 5′ and 3′ ends of the constructs, respectively, for cloning into a standardized dicot protoplast testing vector. The final chimeric promoter that contained the cis-acting regulatory elements are provided as SEQ ID NOs:15-24.

### Example 3: Construct Design

The chimeric cis-acting regulatory element and promoter were tested as a chimeric regulatory element to determine whether the cis-acting regulatory elements could enhance the expression of a downstream coding sequence operably linked to the chimeric cis-acting regulatory element and promoter. As such, the chimeric cis-acting regulatory element and the regulatory element (for example a promoter regulatory element) were cloned upstream of a coding sequence that encoded a fluorescent protein. The standardized dicot protoplast testing vector contains two reporter protein fluorophores, ZsGreen (Matz, M. V., Fradkov, A. F., Labas, Y. A., Zaraisky, A. G., Markelov, M. L., & Lukyanov, S. A. 1999. Fluorescent proteins from the nonbioluminescent Anthozoa species. Nat Biotechnol. 17:969-973.) for measuring the effect of regulatory elements on transcription, and TagRFP (Merzlyak, E. M., Goedhart, J., Shcherbo, D., Bulina, M. E., Shcheglov, A. S. Fradkov, A. F., Gaintzeva, A., Lukyanov, K. A., Lukyanov, S., Gadella, T. W. J. and Chudakov, D. M. 2007. Bright monomeric red fluorescent protein with an extended fluorescence lifetime. Nat Methods. 4:555-557.) for normalization. The ZsGreen coding sequence was driven by the test cassettes as described above. The TagRFP coding sequence was driven by the Arabidopsis thaliana ubiquitin promoter, AT-UBIQ10. The two expression constructs were separated by four terminators, PINII (An, G., Mitra, M., Choi, H. K., Costa, M. A., An, K., Thornburg, R. W. 1989. Functional analysis of the 3′ control region of the potato wound-inducible proteinase inhibitor II gene. Plant Cell. 1:115-122.), W64A (Das et al., 1991), UBQ14 (Mayer et al., 1999) and IN2-1 (Hershey and Stoner, 1991), to prevent read-through transcription. A gene for spectinomycin resistance was included on the vector as a selectable marker for cloning purposes. The bacterial origin of replication of the vector is PUC ORI.

### Example 4: In Planta Testing of the Cis-Acting Regulatory Element

A soybean hypocotyl protoplast assay was used to test the vectors to determine whether the cis-acting regulatory elements could enhance the expression of a downstream coding sequence operably linked to the chimeric cis-acting regulatory element and a regulatory element (for example a promoter regulatory element) as a chimeric regulatory element. Protoplasts were isolated from sliced (0.5-1 mm) hypocotyl tissue from 4-5 day old soy (genotype 93Y21) seedlings and isolated using the solutions and approach as described in Wu and Hanzawa (2018) with the following modifications: 1) 0.75 M mannitol was used in all relevant solutions, 2) vacuum infiltration was 0.5 H, 3) incubation in digestion solution was 2.5 H without agitation, 4) after centrifugation, protoplasts were resuspended in MMg and allowed to rest on ice for 1 H, and 5) protoplasts were not centrifuged after resting, but rather supernatant was removed without disturbing the cell pellet and replaced with fresh MMg. Polyethylene glycol (PEG) transfection of protoplasts was carried out following Wu, F. & Hanzawa, Y. A simple method for isolating of soybean protoplasts and application to transient gene expression analyses. J Vis Exp. 131: e57258. A few modifications were made the to the Wu and Hanzawa protocol: 1) a concentration of 0.75 M mannitol and 2) a concentration of 40% PEG was used in the transfection solution, and 3) a total of 2.5 nM of plasmid DNA was used in the transfection reactions. All transfections were carried out in duplicate. After transfection, protoplasts were plated into 24-well, black glass-bottom plates, and stored at 27° C. in darkness for 16 H. Fluorescent signals emanating from protoplasts were measured using a Cytation5 (BioTek, Winooski, VT). Controls in the assay included an empty vector, i.e., no test cassette, as a background control (EE1906), and mock-treated protoplasts (no DNA). Data collected were at the cellular (object) level, with no fewer than 300 protoplasts counted per vector. In addition to fluorescent signals, the size and circularity of each protoplast was also determined.

### Example 5: Identification of a Cis-Acting Regulatory Element That Increases Expression of a Coding Sequence

To be counted as a ‘protoplast’, the object detected and measured by the Cytation5™ had to 1) to fluoresce red, 2) be between 20-150 μm and 3) have a circularity value equal to or greater than 0.49 (with a rolling ball diameter of 300 μm). For each protoplast, the green fluorescent signal datapoint (emanating from ZsGreen) was divided by its corresponding red fluorescent datapoint (emanating from TagRFP) to normalize for transfection efficiency, and the geometric mean (mean) and standard error of the mean (SEM) was calculated for each vector. The data are reported in “Relative Fluorescent Units” (RFU). An active element is defined as any RFU 4-fold or greater over the background control.

One construct, EE2889, corresponding to the JERECRESTR cis-acting regulatory element (SEQ ID NO:9), exhibited enhanced activity as a modulating element as a chimeric regulatory element (FIG. 1). This cis-acting regulatory element drove robust expression of the fluorescent coding sequence. The remainder of the elements did not show activity, even marginally, demonstrating that not all elements listed in the PLACE database can be used as modulating elements by merely placing them in the regulatory element (for example a promoter regulatory element) of a gene. As such, the JERECRESTR cis-acting regulatory element (SEQ ID NO:9) was exemplified for the first time as a cis-acting regulatory element capable of functioning as a modulating element within a chimeric regulatory element to drive expression of a coding sequence.

### Example 6: Identification of Additional Cis-Acting Regulatory Elements That Increase Expression of a Coding Sequence

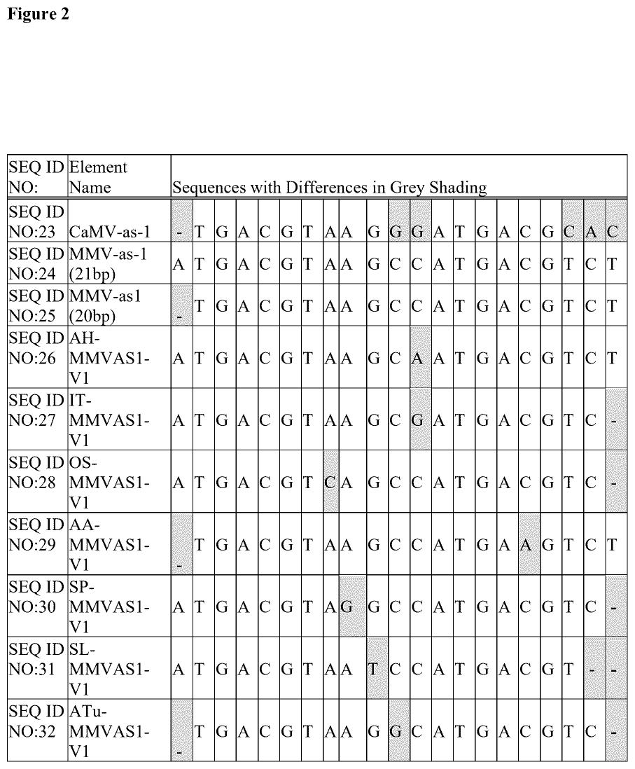

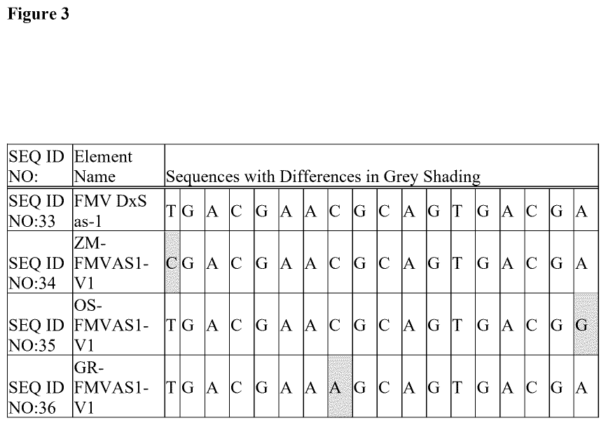

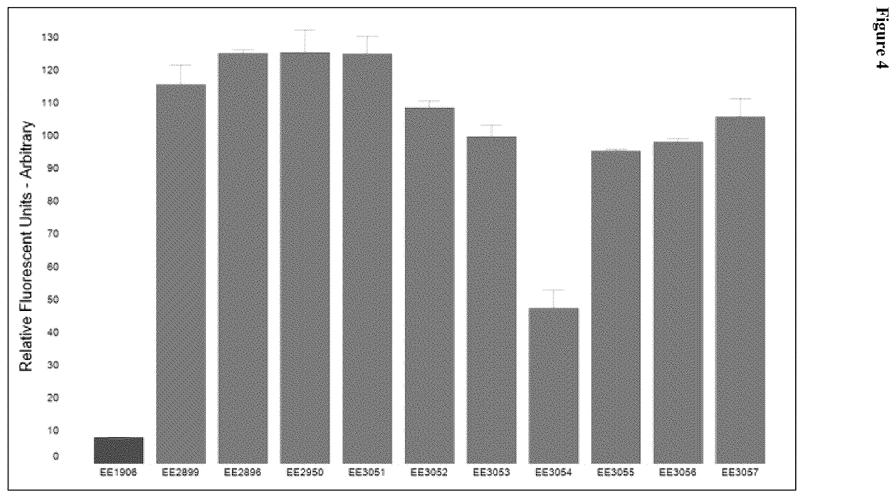

Cis-acting regulatory elements were obtained from the annotated, full-length transcriptional promoters of the figwort mosaic virus (FMV; Sanger, M., Daubert, S. and Goodman, R. M. 1990. Characteristics of a strong promoter from figwort mosaic virus: comparison with the analogous 35S promoter from cauliflower mosaic virus and the regulated mannopine synthase promoter. Plant Mol Biol. 14:433-443.) and the mirabilis mosaic virus (MMV; Dey, N. and Maiti, I. B. 1999. Structure and promoter/leader deletion analysis of mirabilis virus (MMV) full-length transcription promoter in transgenic plants. Plant Mol Biol. 40:771-782.). The cis-acting regulatory elements that were obtained are listed as SEQ ID NO:25 and SEQ ID NO:35 and provided in Tables 2 and 3. Variants of SEQ ID NO:25 were developed and are further provided in Table 2 as SEQ ID NO:26-34. A sequence alignment of SEQ ID NO:25-34 is provided as FIG. 2. Likewise, variants of SEQ ID NO:35 were developed and are further provided in Table 3 as SEQ ID NO:36-38. A sequence alignment of SEQ ID NO:36-38 is provided as FIG. 3. These regulatory elements were engineered upstream of a regulatory element (for example a plant promoter) and used to produce standardized dicot protoplast testing vectors as described in Example 2 and 3. Finally, the cis-acting regulatory elements were tested in a soy hypocotyl protoplast assay as described in Example 4 to test the vectors to determine whether the cis-acting regulatory elements could enhance the expression of a downstream coding sequence operably linked to the chimeric cis-acting regulatory element and another regulatory element (for example a promoter regulatory element) to function as a chimeric regulatory element.

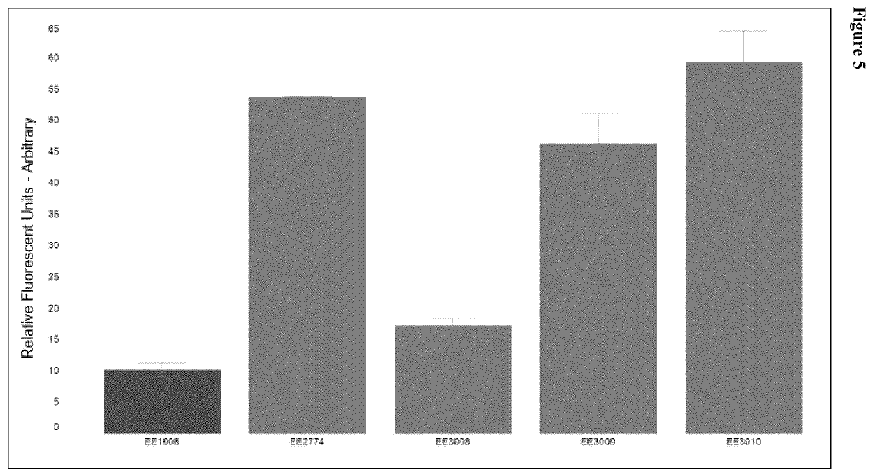

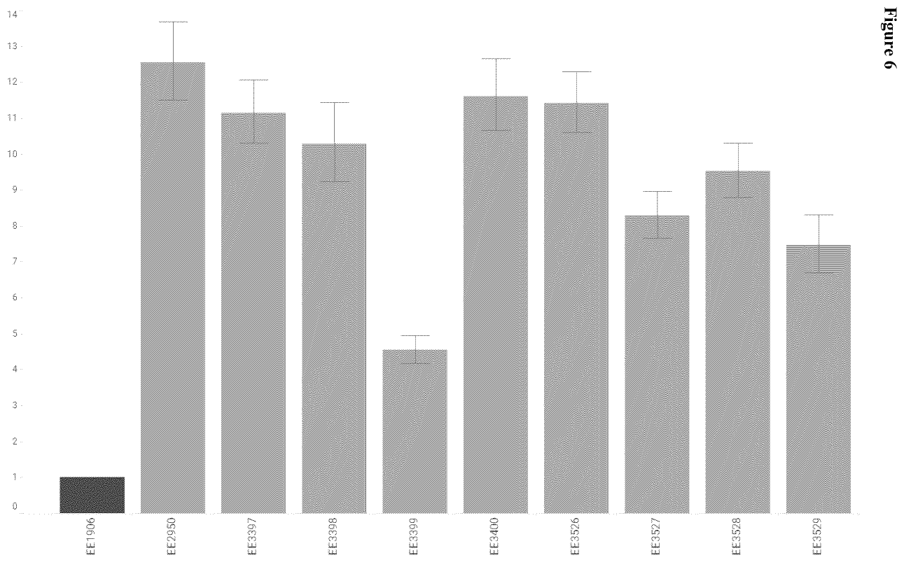

TABLE 2
Elements selected for testing from MMV as-1 and plant-derived variants.

| | | | SEQ | FIG. 4 | RFU | RFU |
|---|---|---|---|---|---|---|
| Element Name | Element Origin | Sequence | ID NO: | Label: | Mean | SEM |
| No element | N/A | N/A | N/A | EE1906 | 8.0 | 0.23 |
| as-1 (20 bp) | CaMV FLt PRO | TGACGTAAGGGATGACGCAC | SEQ ID NO: 25 | EE2899 | 115.8 | 6.05 |
| MMV as-1 | MMV Flt PRO | ATGACGTAAGCCATGACGTCT | SEQ ID NO: 26 | EE2896 | 125.3 | 1.10 |
| MMV as-1 | MMV Flt PRO | TGACGTAAGCCATGACGTCT | SEQ ID NO: 27 | EE2950 | 125.5 | 6.94 |
| (20 bp) | | | | | | |
| AH-MMVAS1-V1 | Arachis hypogaea | ATGACGTAAGCaATGACGTCT | SEQ ID NO: 28 | EE3051 | 125.1 | 5.42 |
| IT-MMVAS1-V1 | Ipomoea triloba | ATGACGTAAGCgATGACGTC | SEQ ID NO: 29 | EE3052 | 108.8 | 2.14 |
| OS-MMVAS1-V1 | Oryza sativa | ATGACGTcAGCCATGACGTC | SEQ ID NO: 30 | EE3053 | 99.8 | 3.64 |
| AA-MMVAS1-V1 | Arabis alpina | TGACGTAAGCCATGAaGTCT | SEQ ID NO: 31 | EE3054 | 47.4 | 5.55 |
| SP-MMVAS1-V1 | Solanum | ATGACGTAgGCCATGACGTC | SEQ ID | EE3055 | 95.4 | 0.48 |
| | pennellii | | NO: 32 | | | |
| SL-MMVAS1-V1 | Solanum | ATGACGTAAtCCATGACGT | SEQ ID | EE3056 | 98.2 | 1.25 |
| | lycopersicum | | NO: 33 | | | |
| ATu-MMVAS1- | Allium | TGACGTAAGgCATGACGTC | SEQ ID | EE3057 | 105.9 | 5.55 |
| V1 | turkestanicum | | NO: 34 | | | |

TABLE 3
Elements selected for testing from FMV as-1 and plant-derived variants.

| | | | SEQ | FIG. 5 | RFU | RFU |
|---|---|---|---|---|---|---|
| Element Name | Element Origin | Sequence | ID NO: | Label: | Mean | SEM |
| FMV DxS as-1 | FMV FLt PRO | TGACGAACGCAGTGACGA | SEQ ID NO: 35 | EE2774 | 53.8 | 0.1 |
| ZM-FMVAS1-V1 | Zea mays | cGACGAACGCAGTGACGA | SEQ ID NO: 36 | EE3008 | 17.2 | 1.3 |
| OS-FMVAS1-V1 | Oryza sativa | TGACGAACGCAGTGACGg | SEQ ID NO: 37 | EE3009 | 46.3 | 4.9 |
| GR-FMVAS1-V1 | Gossypium | TGACGAAaGCAGTGACGA | SEQ ID NO: 38 | EE3010 | 59.3 | 5.1 |
| | raimondii | | | | | |

The results of the expression profiling indicated that the MMV as-1 cis-acting regulatory element (SEQ ID NO:25) and the variants there of (SEQ ID NO:26-34), exhibited superior activity as a modulating element within a chimeric regulatory element (for example a cis-acting regulatory element and a promoter regulatory element) (FIG. 4). This cis-acting regulatory element drove robust expression of the fluorescent coding sequence. Although there was some level of variation in the expression of the fluorescent coding sequence as compared to SEQ ID NO:25, all of the variant sequences expressed higher than the CaMV 35S minimal promoter control. As such, the MMV as-1 cis-acting regulatory element (SEQ ID NO:25) and MMV as-1 plant-derived variant cis-acting regulatory element (SEQ ID NO:26-34) were exemplified for the first time as cis-acting regulatory elements capable of functioning as a modulating element within a chimeric regulatory element (for example a promoter and a cis-acting regulatory element) to drive expression of a coding sequence.

The results of the expression profiling indicated that the FMV as-1 cis-acting regulatory element (SEQ ID NO:35) and the variants there of (SEQ ID NO:36-38), exhibited enhanced activity as a modulating element within a chimeric regulatory element (FIG. 5). This cis-acting regulatory element drove robust expression of the fluorescent coding sequence. Although there was some level of variation in the expression of the fluorescent coding sequence as compared to SEQ ID NO:33, all of the variant sequences expressed higher than the CaMV 35S minimal promoter control. As such, the FMV as-1 cis-acting regulatory element (SEQ ID NO:35) and FMV as-1 plant-derived variant cis-acting regulatory element (SEQ ID NO:36-38) were exemplified for the first time as cis-acting regulatory elements capable of functioning as a modulating element within a chimeric regulatory element (for example a promoter and a cis-acting regulatory element) to drive expression of a coding sequence.

### Example 7: Bioinformatic Based Analysis of Expression Elements to Identify Putative Cis-Genic Expression Elements

Variants for public EME motifs identified (MMV, GVBAV, CSVMV, MMV-1, GM-PSGS3AF1-V3 and JERECRSTR) were scanned in multiple genomes of interest including Zea mays (various genotypes derived form internal pan-genome study were explored A63, CSB8V, ED85E, EDE4N, EECPR, EEPAR, GH61, GR3KP, GR84Z, GRW2Z, HEF3D, HN4CN, HNH9H), Glycine max, Oryza sativa, Sorghum bicolor, Brassica napus. Genome sequences for these were downloaded from internal GAIA repository, NCBI and/or EnsemblPlants.

Sequences obtained from public EMEs motifs were first converted into a position weight matrix (PWM) representing probability of base at the specific position. Once the PWM was obtained for each EME sequence, these were then used to scan the whole genome using Find Individual Motif Occurrences (FIMO). The program computes a log-likelihood ratio score for each position in a given sequence database (whole genome sequences for crop of interest in our case), uses established dynamic programming methods (assuming a zero-order null model in which sequences are generated at random with user-specified per-letter background frequencies) to convert this score to a P-value and then applies false discovery rate analysis (using a bootstrap method proposed by Storey J D. A direct approach to false discovery rates. Journal of the Royal Statistical Society: Series B, 64:479-498, 2002.) to estimate a q-value for each position in the given sequence (Grant C E, Bailey T L, Noble W S. FIMO: Scanning for occurrences of a given motif. Bioinformatics, 27(7):1017-1018, 2011). Output was a ranked list of motif (EME) occurrences in the genome scanned, each with an associated log-likelihood ratio score, P-value and q-value. Top ranked variants were then used for subsequent characterization.

### Example 8: Identification of MMV-1 Soybean Variant Cis-Acting Regulatory Elements That Increase Expression of a Coding Sequence

Cis-acting regulatory elements were obtained by screening the MMV as-1 genetic element (SEQ ID NO:27) against plant genomic sequence databases (e.g., rice, soybean, canola, maize, and sorghum). The screening was completed via a FIMO algorithm from the MEME suite with a cap of the first 100 hits (redundant) (Charles E. Grant, Timothy L. Bailey, and William Stafford Noble, “FIMO: Scanning for occurrences of a given motif”, Bioinformatics, 27(7):1017-1018, 2011). This analysis of the plant genomic sequence database resulted in the identification of a large number or sequences that shared relatively high levels of sequence identity to the MMV as-1 genetic element. The pool of sequences was further culled to identify putative cis-acting regulatory elements for testing in planta. These sequences were sorted and selected based on predicted characteristics of active regulatory elements, which included, for example, the intact bZIP binding motifs (e.g., ‘ACGT’) and sequences that shared high levels of sequence identity to the parent element for testing.

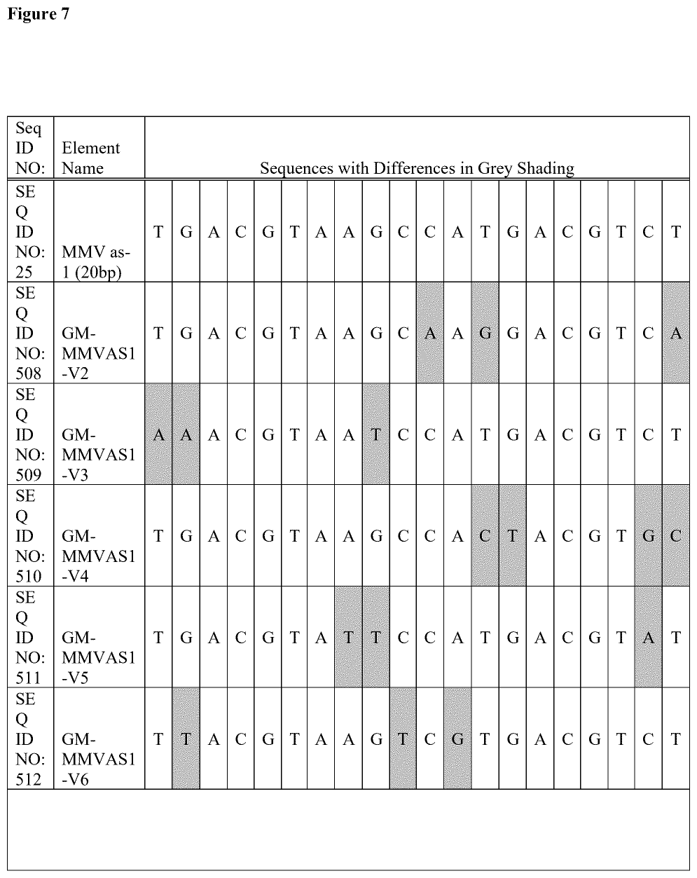

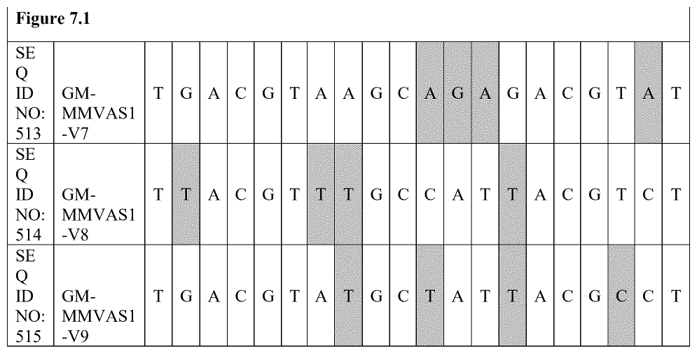

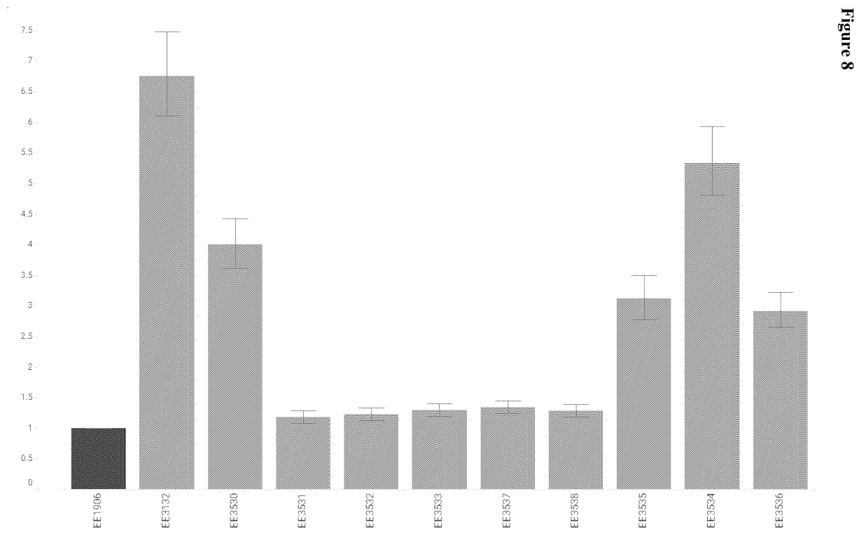

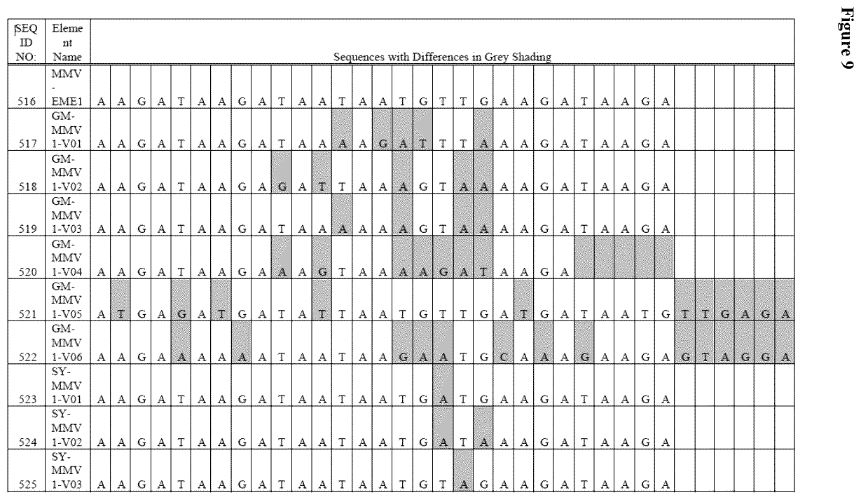

The cis-acting regulatory elements that were obtained are listed as SEQ ID NO:508-SEQ ID NO:515 and provided in Table 4. A sequence alignment of 508-SEQ ID NO:515 is provided as FIG. 7. These regulatory elements were engineered upstream of a regulatory element (for example a plant promoter) and used to produce standardized dicot protoplast testing vectors as described in Example 2 and 3. The cis-acting regulatory elements were incorporated in duplicate within the construct. Those with skill in the art would appreciate that the cis-acting regulatory element could be include as a monomer, dimer, trimer, or in any copy number to drive robust expression of a coding sequence. Finally, the cis-acting regulatory elements were tested in a soy hypocotyl protoplast assay as described in Example 4 to test the vectors to determine whether the cis-acting regulatory elements could enhance the expression of a downstream coding sequence operably linked to the chimeric cis-acting regulatory element and another regulatory element (for example a promoter regulatory element) to function as a chimeric regulatory element.

TABLE 4
Elements selected for testing from MMV as-1 and plant-derived variants.

| Element | Element | | SEQ | FIG. 6 | Fold | Fold 95% | Fold 95% |
|---|---|---|---|---|---|---|---|
| Name | Origin | Sequence | ID NO: | Label: | Change | Lower CI | Upper CI |
| No Element | N/A | N/A | N/A | EE1906 | 1.0 | N/A | N/A |
| MMV as-1 | CaMV FLt PRO | TGACGTAAGCCATGACGTCT | SEQ ID | EE2950 | 12.6 | 11.5 | 13.7 |
| (20 bp) | | | NO: 27 | | | | |
| GM-MMVAS1- | Glycine max | TGACGTAAGCAAGGACGTCA | SEQ ID | EE3397 | 11.2 | 10.3 | 12.1 |
| V02 | | | NO: 508 | | | | |
| GM-MMVAS1- | Glycine max | AAACGTAATCCATGACGTCT | SEQ ID | EE3398 | 10.3 | 9.2 | 11.4 |
| V03 | | | NO: 509 | | | | |
| GM-MMVAS1- | Glycine max | TGACGTAAGCCACTACGTGC | SEQ ID | EE3399 | 4.6 | 4.2 | 5.0 |
| V04 | | | NO: 510 | | | | |
| GM-MMVAS1- | Glycine max | TGACGTATTCCATGACGTAT | SEQ ID | EE3400 | 11.6 | 10.7 | 12.7 |
| V05 | | | NO: 511 | | | | |
| GM-MMVAS1- | Glycine max | TTACGTAAGTCGTGACGTCT | SEQ ID | EE3526 | 11.4 | 10.6 | 12.3 |
| V06 | | | NO: 512 | | | | |
| GM-MMVAS1- | Glycine max | TGACGTAAGCAGAGACGTAT | SEQ ID | EE3527 | 8.3 | 7.7 | 8.9 |
| V07 | | | NO: 513 | | | | |
| GM-MMVAS1- | Glycine max | TTACGTTTGCCATTACGTCT | SEQ ID | EE3528 | 9.5 | 8.0 | 10.3 |
| V08 | | | NO: 514 | | | | |
| GM-MMVAS1- | Glycine max | TGACGTATGCTATTACGCCT | SEQ ID | EE3529 | 7.5 | 6.7 | 18.3 |
| V09 | | | NO: 515 | | | | |

The results of the expression profiling indicated that the MMV as-1 cis-acting regulatory element (SEQ ID NO:27) and the variants obtained from soybean (SEQ ID NO:508-515), exhibited activity as a modulating element within a chimeric regulatory element (for example a cis-acting regulatory element and a promoter regulatory element) (FIG. 6). This cis-acting regulatory element drove robust expression of the fluorescent coding sequence in soybean protoplasts. Those with skill in the art would appreciate that these cis-acting regulatory elements would also drive robust expression of a coding sequence in other plants. Although there was some level of variation in the expression of the fluorescent coding sequence as compared to SEQ ID NO:27, all of the variant sequences expressed higher than the CaMV 35S minimal promoter control. As such, the MMV as-1 cis-acting regulatory element (SEQ ID NO:27) and MMV as-1 plant-derived variant cis-acting regulatory element (SEQ ID NO:508-515) were exemplified for the first time as cis-acting regulatory elements capable of functioning as a modulating element within a chimeric regulatory element (for example a promoter and a cis-acting regulatory element) to drive expression of a coding sequence.

### Example 9: Identification of Synthetic MMV-1 Variant Cis-Acting Regulatory Elements and Synthetic G-Box Binding Motif Cis-Acting Regulatory Elements That Increase Expression of a Coding Sequence

Cis-acting regulatory elements were obtained by analyzing the MMV-1 promoter. Initially, 70 base pair fragments of the MMV-1 promoter were overlapped with one another to generate a consensus sequence. The sequences covered 360 base pairs of the MMV-1 promoter core, and the sequences overlapped by 35 base pairs. The resulting sequences were tested in planta to determine if any of the putative cis-acting regulatory elements could be used to robustly drive expression. One fragment demonstrated above background expression levels and was selected for further characterization. The region of activity was narrowed down to a 29 base pair element, and was named MMV-EMEI (SEQ ID NO:516). The identified MMV-EME1 sequence was used to obtain variant sequences from the maize, soy, canola, rice and sorghum genomes using bioinformatic analysis methods similar to those described in Example 7.

The cis-acting regulatory elements that were obtained are listed as SEQ ID NO:516-SEQ ID NO:525 and provided in Table 5. A sequence alignment of SEQ ID NO:516-SEQ ID NO:525 is provided as FIG. 9. These regulatory elements were engineered upstream of a regulatory element (for example a plant promoter) and used to produce standardized dicot protoplast testing vectors as described in Example 2 and 3. The cis-acting regulatory elements were incorporated in duplicate within the construct. Those with skill in the art would appreciate that the cis-acting regulatory element could be include as a monomer, dimer, trimer, or in any copy number to drive robust expression of a coding sequence. Finally, the cis-acting regulatory elements were tested in a soy hypocotyl protoplast assay as described in Example 4 to test the vectors to determine whether the cis-acting regulatory elements could enhance the expression of a downstream coding sequence operably linked to the chimeric cis-acting regulatory element and another regulatory element (for example a promoter regulatory element) to function as a chimeric regulatory element.

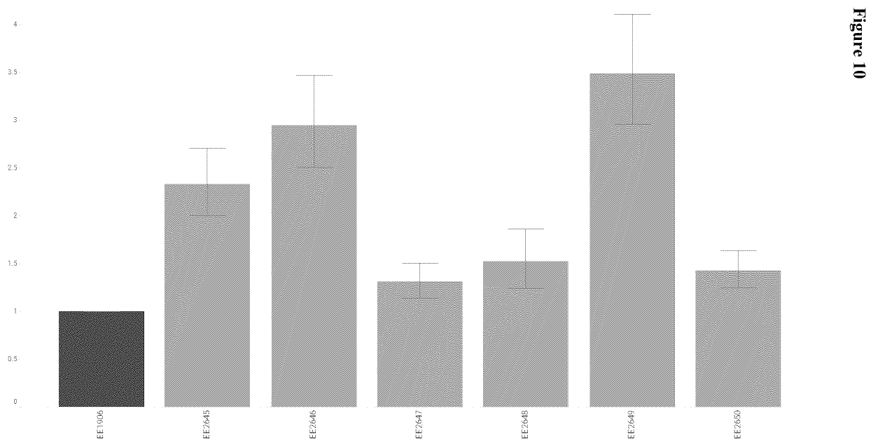

TABLE 5
Elements selected for testing.

| | | | | | | Fold | Fold |
|---|---|---|---|---|---|---|---|
| | | | | | | 95% | 95% |
| Element | Element | | SEQ | FIG. 8 | Fold | Lower | Upper |
| Name | Origin | Sequence | ID NO: | Label: | Change | CI | CI |
| No Element | N/A | N/A | N/A | EE1906 | 1.0 | N/A | N/A |
| MMV-EME1 | MMV FLt PRO | AAGATAAGATAATAATGTTGAAGATAAGA | SEQ ID | EE3132 | 6.8 | 0.7 | 0.7 |
| | | | NO: 516 | | | | |
| GM-MMV1-V01 | Glycine max | AAGATAAGATAAAAGATTTAAAGATAAGA | SEQ ID | EE3530 | 14.0 | 0.4 | 0.4 |
| | | | NO: 517 | | | | |
| GM-MMV1-V02 | Glycine max | AAGATAAGAGATTAAAGTAAAAGATAAGA | SEQ ID | EE3531 | 1.2 | 0.1 | 0.1 |
| | | | NO: 518 | | | | |
| GM-MMV1-V03 | Glycine max | AAGATAAGATAAAAAAGTAAAAGATAAGA | SEQ ID | EE3532 | 1.2 | 0.1 | 0.1 |
| | | | NO: 519 | | | | |
| GM-MMV1-V04 | Glycine max | AAGATAAGAAAGTAAAAGATAAGA | SEQ ID | EE3533 | 1.3 | 0.1 | 0.1 |
| | | | NO: 520 | | | | |
| GM-MMV1-V05 | Glycine max | ATGAGATGATATTAATGTTGATGATAATGT | SEQ ID | EE3537 | 1.3 | 0.1 | 0.1 |
| | | TGAGA | NO: 521 | | | | |
| GM-MMV1-V06 | Glycine max | AAGAAAAAATAATAAGAATGCAAAGAAGA | SEQ ID | EE3538 | 1.3 | 0.1 | 0.1 |
| | | GTAGGA | NO: 522 | | | | |
| SY-MMV1-V01 | Sythethic | AAGATAAGATAATAATGATGAAGATAAGA | SEQ ID | EE3535 | 3.1 | 10.3 | 0.4 |
| | | | NO:523 | | | | |
| SY-MMV1-V02 | Synthetic | AAGATAAGATAATAATGATAAAGATAAGA | SEQ ID | EE3534 | 5.3 | 0.5 | 0.6 |
| | | | NO: 524 | | | | |
| SY-MMV1-V03 | Synthetic | AAGATAAGATAATAATGTAGAAGATAAGA | SEQ ID | EE3536 | 2.9 | 0.3 | 0.3 |
| | | | NO: 525 | | | | |

The results of the expression profiling indicated that the MMV-EMEI cis-acting regulatory element (SEQ ID NO:516) and a number of the other variants (SEQ ID NO:517, and SEQ ID NO:523-525), exhibited activity as a modulating element within a chimeric regulatory element (for example a cis-acting regulatory element and a promoter regulatory element) (FIG. 8). This cis-acting regulatory element drove robust expression of the fluorescent coding sequence in soybean protoplasts. Those with skill in the art would appreciate that these cis-acting regulatory elements would also drive robust expression of a coding sequence in other plants. Although there was some level of variation in the expression of the fluorescent coding sequence as compared to SEQ ID NO:516, all of the variant sequences expressed higher than the CaMV 35S minimal promoter control. As such, the MMV-EMEI cis-acting regulatory element (SEQ ID NO:516) and a number of the other variants (SEQ ID NO:517, and SEQ ID NO:523-525) were exemplified for the first time as cis-acting regulatory elements capable of functioning as a modulating element within a chimeric regulatory element (for example a promoter and a cis-acting regulatory element) to drive expression of a coding sequence.

### Example 10: Identification of PS-GS3A Variant Cis-Acting Regulatory Elements That Increase Expression of a Coding Sequence

Cis-acting regulatory elements were obtained by analyzing the regulatory elements of PS-GS3A that was obtained from the pea glutamine synthetase GS3A promoter (Brears, T., Walker, E. L., Coruzzi, G. M. A promoter sequence involved in cell-specific expression of the pea gluamine syntetas GS3A gene in organs of transgenic tobacco and alfalfa. Plant J. 1(2) 235-244). Cis-acting regulatory elements were obtained by screening the PS-GS3A genetic element (SEQ ID NO:526) against plant genomic sequence databases from soybean. using bioinformatic analysis methods similar to those described in Example 7. This analysis of the plant genomic sequence database resulted in the identification of a large number or sequences that shared relatively high levels of sequence identity to the MMV as-1 genetic element. The pool of sequences was further culled to identify putative cis-acting regulatory elements for testing in planta. These sequences were sorted and selected based on predicted characteristics of active regulatory elements, which included, for example, the intact bZIP binding motifs (e.g., ‘ACGT’) and sequences that shared high levels of sequence identity to the parent element for testing. The identified sequences were tested in planta to determine if any of the putative cis-acting regulatory elements could be used to robustly drive expression.

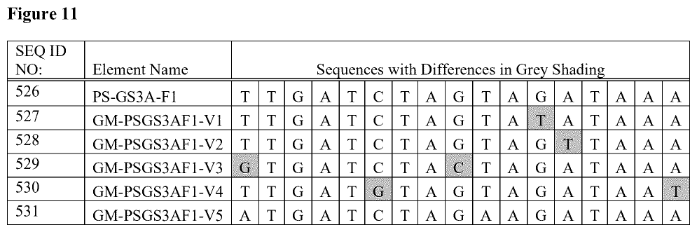

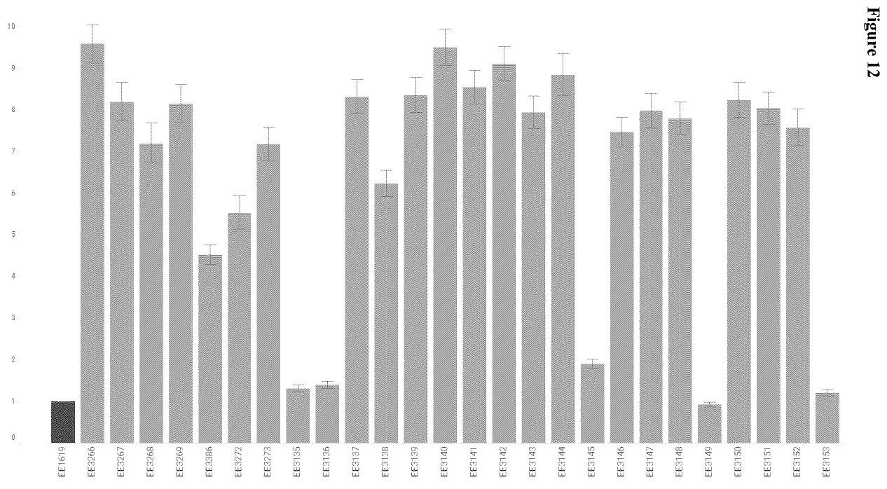

The cis-acting regulatory elements that were obtained are listed as SEQ ID NO:526-SEQ ID NO:531 and provided in Table 6. A sequence alignment of SEQ ID NO:526-SEQ ID NO:531 is provided as FIG. 11. These regulatory elements were engineered upstream of a regulatory element (for example a plant promoter) and used to produce standardized dicot protoplast testing vectors as described in Example 2 and 3. The cis-acting regulatory elements were incorporated in duplicate within the construct. Those with skill in the art would appreciate that the cis-acting regulatory element could be include as a monomer, dimer, trimer, or in any copy number to drive robust expression of a coding sequence. Finally, the cis-acting regulatory elements were tested in a soy hypocotyl protoplast assay as described in Example 4 to test the vectors to determine whether the cis-acting regulatory elements could enhance the expression of a downstream coding sequence operably linked to the chimeric cis-acting regulatory element and another regulatory element (for example a promoter regulatory element) to function as a chimeric regulatory element.

TABLE 6
Elements selected for testing.

| | | | | | | Fold | Fold |
|---|---|---|---|---|---|---|---|
| | | | | | | 95% | 95% |
| Element | Element | | SEQ | FIG. 10 | Fold | Lower | Upper |
| Name | Origin | Sequence | ID NO: | Label: | Change | CI | CI |
| No Element | N/A | N/A | N/A | EE1906 | 1.0 | N/A | N/A |
| PS-GS3A-F1 | Pisum | TTGATCTAGTAGATAAA | SEQ ID NO: 526 | EE2645 | 3.0 | 2.5 | 3.5 |
| | sativum | | | | | | |
| GM-PSGS3AF1- | Glycine | TTGATCTAGTATATAAA | SEQ ID NO: 527 | EE2646 | 1.3 | 1.1 | 1.5 |
| V1 | max | | | | | | |
| GM-PSGS3AF1- | Glycine | TTGATCTAGTAGTTAAA | SEQ ID NO: 528 | EE2647 | 1.5 | 1.2 | 1.9 |
| V2 | max | | | | | | |
| GM-PSGS3AF1- | Glycine | GTGATCTACTAGATAAA | SEQ ID NO: 529 | EE2648 | 3.5 | 3.0 | 4.1 |
| V3 | max | | | | | | |
| GM-PSGS3AF1- | Glycine | TTGATGTAGTAGATAAT | SEQ ID NO: 530 | EE2649 | 1.4 | 1.3 | 1.6 |
| V4 | max | | | | | | |
| GM-PSGS3AF1- | Glycine | ATGATCTAGAAGATAAA | SEQ ID NO: 531 | EE2650 | 1.9 | 1.7 | 2.2 |
| V5 | max | | | | | | |

The results of the expression profiling indicated that the PS-GS3A-F1 cis-acting regulatory element (SEQ ID NO:526) and a number of the soybean variants (SEQ ID NO:527-531), exhibited activity as a modulating element within a chimeric regulatory element (for example a cis-acting regulatory element and a promoter regulatory element) (FIG. 10). This cis-acting regulatory element drove robust expression of the fluorescent coding sequence in soybean protoplasts. Those with skill in the art would appreciate that these cis-acting regulatory elements would also drive robust expression of a coding sequence in other plants. Although there was some level of variation in the expression of the fluorescent coding sequence as compared to the PS-GS3A-F1 cis-acting regulatory element (SEQ ID NO:526), all of the variant sequences expressed higher than SEQ ID NO:526. As such, the soybean variants (SEQ ID NO:517, and SEQ ID NO:527-531) were exemplified for the first time as cis-acting regulatory elements capable of functioning as a modulating element within a chimeric regulatory element (for example a promoter and a cis-acting regulatory element) to drive expression of a coding sequence.

### Example 11: Identification of Cis-Acting Regulatory Elements That Increase Expression of a Coding Sequence From Virus

Cis-acting regulatory elements were obtained by screening the CaMV as-1 genetic element against viral genomic sequence databases using bioinformatic analysis methods similar to those described in Example 7. This analysis of the plant genomic sequence database resulted in the identification of a large number or sequences that shared relatively high levels of sequence identity to the CaMV as-1 genetic element. The pool of sequences was further culled to identify putative cis-acting regulatory elements for testing in planta.

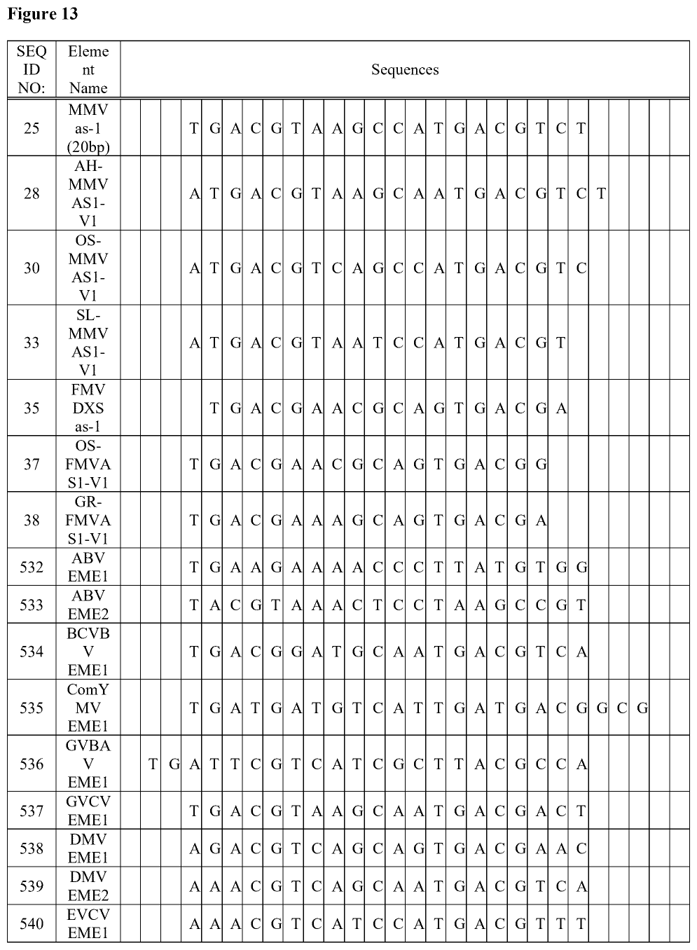

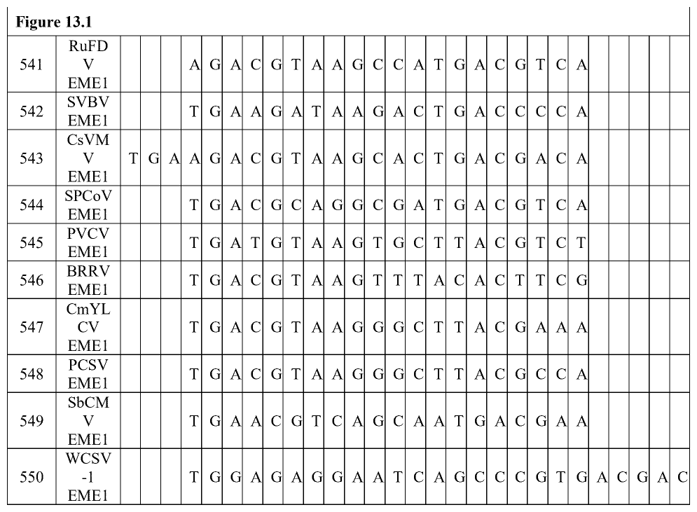

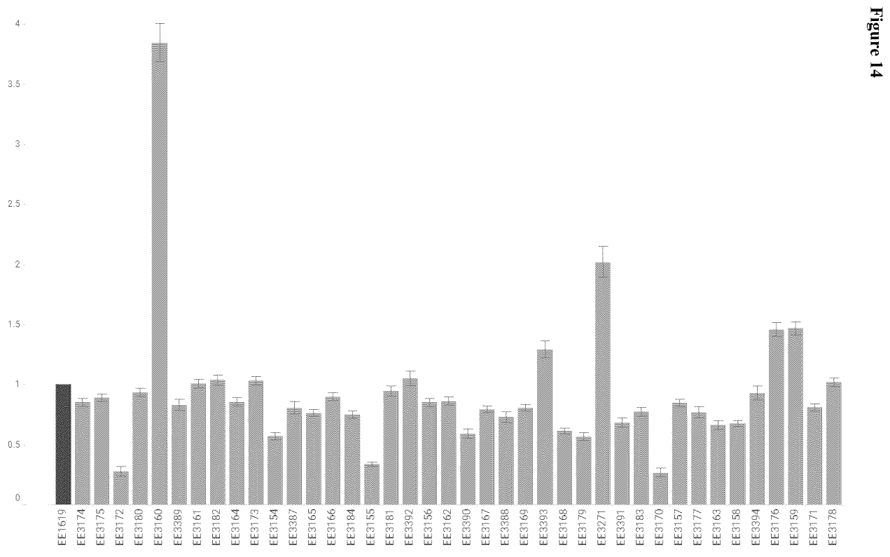

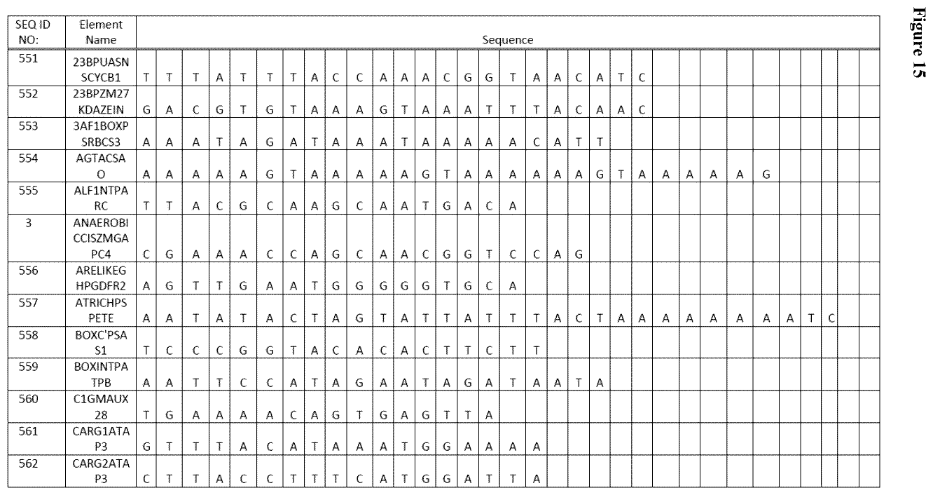

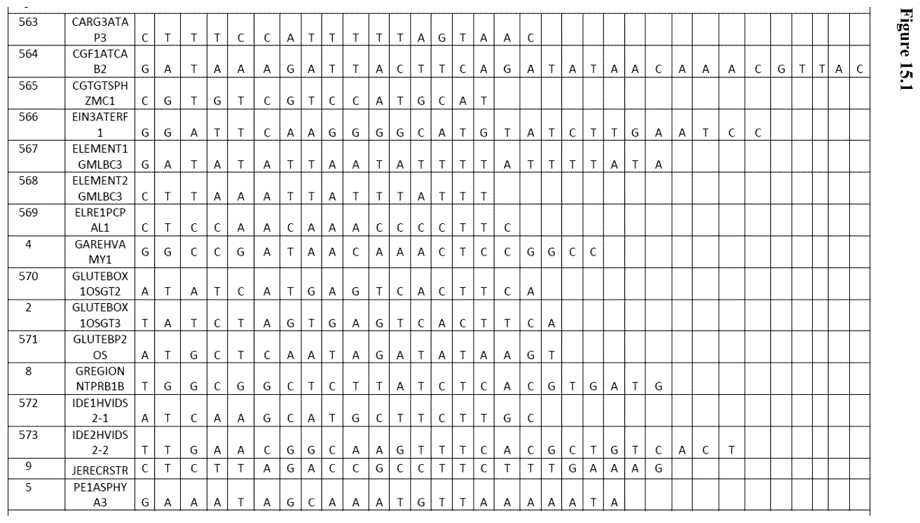

The cis-acting regulatory elements that were obtained are listed as SEQ ID NO:532-SEQ ID NO:550 and provided in Table 7. A sequence alignment of SEQ ID NO:526-SEQ ID NO:531 is provided as FIG. 13. These regulatory elements were engineered upstream of a regulatory element (for example a plant promoter) and used to produce standardized monocot protoplast testing vectors as described in Example 2 and 3, where the monocot promoter (Zea mays GOS2 promoter) was incorporated into construct design to drive expression in Zea mays protoplasts. The cis-acting regulatory elements were incorporated in duplicate within the construct. Those with skill in the art would appreciate that the cis-acting regulatory element could be include as a monomer, dimer, trimer, or in any copy number to drive robust expression of a coding sequence. Finally, the cis-acting regulatory elements were tested in a transient gene expression Zea mays leaf mesophyll protoplast platform as described in Examples 1 and 3 of patent application WO2018183878A1. The analysis was completed to test the vectors to determine whether the cis-acting regulatory elements could enhance the expression of a downstream coding sequence operably linked to the chimeric cis-acting regulatory element and another regulatory element (for example a promoter regulatory element) to function as a chimeric regulatory element.

TABLE 7
Elements selected for testing.

| | | | | | | Fold | Fold |
|---|---|---|---|---|---|---|---|
| | | | | | | 95% | 95% |
| Element | | | SEQ | FIG. 12 | Fold | Lower | Upper |
| Name | Element Origin | Sequence | ID NO: | Label: | Change | CI | CI |
| No Element | N/A | N/A | N/A | EE1619 | 1.0 | N/A | N/A |
| MMV as-1 | MMV FLt PRO | TGACGTAAGCCATGACGTCT | SEQ ID | EE3266 | 9.6 | 9.1 | 10.0 |
| (20 bp) | | | NO: 27 | | | | |
| AH-MMVAS1- | Arachis hypogaea | ATGACGTAAGCAATGACGTCT | SEQ ID | EE3267 | 8.2 | 7.7 | 8.7 |
| V1 | | | NO: 28 | | | | |
| OS-MMVAS1- | Oryza sativa | ATGACGTCAGCCATGACGTC | SEQ ID | EE3268 | 7.2 | 6.7 | 7.7 |
| V1 | | | NO: 30 | | | | |
| SL-MMVAS1- | Solanum | ATGACGTAATCCATGACGT | SEQ ID | EE3269 | 8.1 | 7.7 | 8.6 |
| V1 | lycopersicum | | NO: 33 | | | | |
| FMV DXS as-1 | FMV FLt PRO | TGACGAACGCAGTGACGA | SEQ ID | EE3386 | 4.5 | 4.3 | 4.8 |
| | | | NO: 35 | | | | |
| OS-FMVAS1- | Oryza sativa | TGACGAACGCAGTGACGG | SEQ ID | EE3272 | 5.5 | 5.1 | 5.9 |
| V1 | | | NO: 37 | | | | |
| GR-FMVAS1- | Gossypium | TGACGAAAGCAGTGACGA | SEQ ID | EE3273 | 7.2 | 6.8 | 7.6 |
| V1 | raimondii | | NO: 38 | | | | |
| ABV EME1 | Aglaonema | TGAAGAAAACCCTTATGTGG | SEQ ID | EE3135 | 1.3 | 1.2 | 1.4 |
| | bacilliform virus | | NO: 532 | | | | |
| ABV EME2 | Aglaonema | TACGTAAACTCCTAAGCCGT | SEQ ID | EE3136 | 1.4 | 1.3 | 1.5 |
| | bacilliform virus | | NO: 533 | | | | |
| BCVBV EME1 | Bougianvillea | TGACGGATGCAATGACGTCA | SEQ ID | EE3137 | 8.3 | 7.9 | 8.7 |
| | chlorotic vein | | NO: 534 | | | | |
| | banding virus | | | | | | |
| ComYMV | Commelina yellow | TGATGATGTCATTGATGACGGCG | SEQ ID | EE3138 | 6.2 | 5.9 | 6.6 |
| EME1 | mottle virus | | NO: 535 | | | | |
| GVBAV EME1 | Gooseberry vein | TGATTCGTCATCGCTTACGCCA | SEQ ID | EE3139 | 8.4 | 7.9 | 8.8 |
| | banding | | NO: 536 | | | | |
| | associated virus | | | | | | |
| GVCV EME1 | Grapevine | TGACGTAAGCAATGACGACT | SEQ ID | EE3140 | 9.5 | 9.1 | 9.9 |
| | clearning virus | | NO: 537 | | | | |
| DMV EME1 | Dahlia mosaic | AGACGTCAGCAGTGACGAAC | SEQ ID | EE3141 | 8.5 | 8.2 | 9.0 |
| | virus | | NO: 538 | | | | |
| DMV EME2 | Dahlia mosaic | AAACGTCAGCAATGACGTCA | SEQ ID | EE3142 | 9.1 | 8.7 | 9.5 |
| | Virus | | NO: 539 | | | | |
| EVCV EME1 | Eupatorium vein- | AAACGTCATCCATGACGTTT | SEQ ID | EE3143 | 7.9 | 7.5 | 8.3 |
| | clearing virus | | NO: 540 | | | | |
| RuFDV EME1 | Rudbeckia flower | AGACGTAAGCCATGACGTCA | SEQ ID | EE3144 | 8.8 | 8.3 | 9.4 |
| | distortion virus | | NO: 541 | | | | |
| SVBV EME1 | Strawberry vein | TGAAGATAAGACTGACCCCA | SEQ ID | EE3145 | 1.9 | 1.8 | 2.0 |
| | banding virus | | NO: 542 | | | | |
| CsVMV EME1 | Cassava vein | TGAAGACGTAAGCACTGACGACA | SEQ ID | EE3146 | 7.5 | 7.1 | 7.8 |
| | mosaic virus | | NO: 543 | | | | |
| SPCOV EME1 | Sweet potato | TGACGCAGGCGATGACGTCA | SEQ ID | EE3147 | 8.0 | 7.6 | 8.4 |
| | collusive virus | | NO: 544 | | | | |
| PVCV EME1 | Petunia vein | TGATGTAAGTGCTTACGTCT | SEQ ID | EE3148 | 7.8 | 7.4 | 8.2 |
| | clearing virus | | NO: 545 | | | | |
| BRRV EME1 | Blueberry red | TGACGTAAGTTTACACTTCG | SEQ ID | EE3149 | 0.9 | 0.9 | 1.0 |
| | ringspot virus | | NO: 546 | | | | |
| CmYLCV | Cestrum yellow | TGACGTAAGGGCTTACGAAA | SEQ ID | EE3150 | 8.2 | 7.8 | 8.7 |
| EME1 | leaf curling virus | | NO: 547 | | | | |
| PCSV EME1 | Peanut chlorotic | TGACGTAAGGGCTTACGCCA | SEQ ID | EE3151 | 8.0 | 7.7 | 8.4 |
| | streak virus | | NO: 548 | | | | |
| SbCMV EME1 | Soybean chlorotic | TGAACGTCAGCAATGACGAA | SEQ ID | EE3152 | 7.6 | 7.1 | 8.0 |
| | mottle virus | | NO: 549 | | | | |
| WCSV-1 EME1 | Water chestnut | TGGAGAGGAATCAGCCCGTGACGAC | SEQ ID | EE3153 | 1.2 | 1.1 | 1.3 |
| | soymovirus 1 | | NO: 550 | | | | |

The results of the expression profiling indicated that the CaMV as-1 variants (SEQ ID NO:532-550), exhibited activity as a modulating element within a chimeric regulatory element (for example a cis-acting regulatory element and a promoter regulatory element) (FIG. 12). This cis-acting regulatory element drove robust expression of the fluorescent coding sequence. Likewise, the plant FMV and MMV-1 variants (SEQ ID NO:28-33 and SEQ ID NO:37-38) exhibited activity as a modulating element within a chimeric regulatory element (for example a cis-acting regulatory element and a promoter regulatory element) within maize protoplasts (FIG. 12). Those with skill in the art would appreciate that these cis-acting regulatory elements would also drive robust expression of a coding sequence in other plants. As such, SEQ ID NO:532-550, SEQ ID NO:28-33 and SEQ ID NO:37-38 were exemplified for the first time as cis-acting regulatory elements capable of functioning as a modulating element within a chimeric regulatory element (for example a promoter and a cis-acting regulatory element) to drive expression of a coding sequence.

### Example 12: Identification of Cis-Acting Regulatory Elements That Increase Expression of a Coding Sequence From the PLACE Database

Cis-acting regulatory elements were obtained by screening the PLACE database as previously described in Example 1. This analysis of the plant genomic sequence database resulted in the identification of a large number or sequences to be tested in maize protoplasts. The pool of sequences was further culled to identify putative cis-acting regulatory elements for testing in planta.

The cis-acting regulatory elements that were obtained are listed as SEQ ID NO:551-SEQ ID NO:583 and provided in Table 8. A sequence alignment of SEQ ID NO:551-SEQ ID NO:583 is provided as FIG. 15. These regulatory elements, and SEQ ID NO:2-5, and SEQ ID NO:8-10, were engineered upstream of a regulatory element (for example a plant promoter) and used to produce standardized monocot protoplast testing vectors as described in Example 2 and 3, where the monocot promoter (Zea mays GOS2 promoter) was incorporated into construct design to drive expression in Zea mays protoplasts. The cis-acting regulatory elements were incorporated in duplicate within the construct. Those with skill in the art would appreciate that the cis-acting regulatory element could be include as a monomer, dimer, trimer, or in any copy number to drive robust expression of a coding sequence. Finally, the cis-acting regulatory elements were tested in a transient gene expression Zea mays leaf mesophyll protoplast platform as described in Examples 1 and 3 of patent application WO2018183878A1. The analysis was completed to test the vectors to determine whether the cis-acting regulatory elements could enhance the expression of a downstream coding sequence operably linked to the chimeric cis-acting regulatory element and another regulatory element (for example a promoter regulatory element) to function as a chimeric regulatory element.

TABLE 8
Elements selected for testing.

| | | | | | | Fold | Fold |
|---|---|---|---|---|---|---|---|
| | Database | | | FIG. | | 95% | 95% |
| | Entry | | SEQ ID | 14 | Fold | Lower | Upper |
| Element Name | Number | Sequence | NO: | Label: | Change | CI | CI |
| No element | N/A | N/A | N/A | EE1619 | 1 | N/A | N/A |
| 23BPUASNSCYCB1 | S000283 | TTTATTTACCAAACGGTAACATC | SEQ ID | EE3174 | 0.85 | 0.82 | 0.89 |
| | | | NO: 551 | | | | |
| 23BPZM27KDAZEIN | S000341 | GACGTGTAAAGTAAATTTACAAC | SEQ ID | EE3175 | 0.89 | 10.86 | 0.92 |
| | | | NO: 552 | | | | |
| 3AF1BOXPSRBCS3 | S000004 | AAATAGATAAATAAAAACATT | SEQ ID | EE3172 | 0.28 | 0.24 | 0.32 |
| | | | NO: 553 | | | | |
| AGTACSAO | S000258 | AAAAAGTAAAAAGTAAAAAAGTAAAAAG | SEQ ID | EE3180 | 0.94 | 0.90 | 0.97 |
| | | | NO: 554 | | | | |
| ALFINTPARC | S000238 | TTACGCAAGCAATGACA | SEQ ID | EE3160 | 3.84 | 3.68 | 4.01 |
| | | | NO: 555 | | | | |
| ANAEROBICCISZMGAPC4 | S000350 | CGAAACCAGCAACGGTCCAG | SEQ ID | EE3389 | 0.83 | 0.79 | 0.88 |
| | | | NO: 3 | | | | |
| ARELIKEGHPGDFR2 | S000437 | AGTTGAATGGGGGTGCA | SEQ ID | EE3161 | 1.01 | 0.97 | 1.05 |
| | | | NO: 556 | | | | |
| ATRICHPSPETE | S000248 | AATATACTAGTATTATTTACTAAAAAAA | SEQ ID | EE3182 | 1.04 | 1.00 | 1.08 |
| | | ATC | NO: 557 | | | | |
| BOXC'PSAS1 | S000227 | TCCCGGTACACACTTCTT | SEQ ID | EE3164 | 0.86 | 0.83 | 0.89 |
| | | | NO: 558 | | | | |
| BOXINTPATPB | S000295 | AATTCCATAGAATAGATAATA | SEQ ID | EE3173 | 1.03 | 1.00 | 1.07 |
| | | | NO: 559 | | | | |
| CIGMAUX28 | S000326 | TGAAAACAGTGAGTTA | SEQ ID | EE3154 | 0.57 | 0.54 | 0.60 |
| | | | NO: 560 | | | | |
| CARG1ATAP3 | S000347 | GTTTACATAAATGGAAAA | SEQ ID | EE3387 | 0.80 | 0.75 | 0.86 |
| | | | NO: 561 | | | | |
| CARG2ATAP3 | S000348 | CTTACCTTTCATGGATTA | SEQ ID | EE3165 | 0.76 | 0.74 | 0.79 |
| | | | NO: 562 | | | | |
| CARG3ATAP3 | S000349 | CTTTCCATTTTTAGTAAC | SEQ ID | EE3166 | 0.90 | 0.87 | 0.93 |
| | | | NO: 563 | | | | |
| CGFIATCAB2 | S000213 | GATAAAGATTACTTCAGATATAACAAA | SEQ ID | EE3184 | 0.75 | 0.72 | 0.78 |
| | | CGTTAC | NO: 564 | | | | |
| CGTGTSPHZMC1 | S000294 | CGTGTCGTCCATGCAT | SEQ ID | EE3155 | 0.34 | 0.32 | 0.36 |
| | | | NO: 565 | | | | |
| EIN3ATERF1 | S000332 | GGATTCAAGGGGCATGTATCTTGAATCC | SEQ ID | EE3181 | 0.95 | 0.91 | 0.99 |
| | | | NO: 566 | | | | |
| ELEMENT1GMLBC3 | S000319 | GATATATTAATATTTTATTTTATA | SEQ ID | EE3392 | 1.05 | 0.99 | 1.11 |
| | | | NO: 567 | | | | |
| ELEMENT2GMLBC3 | S000320 | CTTAAATTATTTATTT | SEQ ID | EE3156 | 0.85 | 0.82 | 0.89 |
| | | | NO: 568 | | | | |
| ELREIPCPALI | S000306 | CTCCAACAAACCCCTTC | SEQ ID | EE3162 | 10.86 | 0.83 | 0.89 |
| | | | NO: 569 | | | | |
| GAREHVAMY1 | S000038 | GGCCGATAACAAACTCCGGCC | SEQ ID | EE3390 | \|0.59 | 0.56 | 0.63 |
| | | | NO: 4 | | | | |
| GLUTEBOX10SGT2 | S000046 | ATATCATGAGTCACTTCA | SEQ ID | EE3167 | 0.79 | 0.77 | 0.82 |
| | | | NO: 570 | | | | |
| GLUTEBOX1OSGT3 | S000128 | TATCTAGTGAGTCACTTCA | SEQ ID | EE3388 | \|0.73 | 0.68 | 0.78 |
| | | | NO: 2 | | | | |
| GLUTEBP2OS | S000049 | ATGCTCAATAGATATAAGT | SEQ ID | EE3169 | 0.81 | 0.78 | 0.84 |
| | | | NO: 571 | | | | |
| GREGIONNTPRB1B | S000364 | TGGCGGCTCTTATCTCACGTGATG | SEQ ID | EE3393 | 1.29 | 1.22 | 1.36 |
| | | | NO: 8 | | | | |
| IDEIHVIDS2 | S000463 | ATCAAGCATGCTTCTTGC | SEQ ID | EE3168 | 0.61 | 0.59 | 0.64 |
| | | | NO: 572 | | | | |
| IDE2HVIDS2 | S000464 | TTGAACGGCAAGTTTCACGCTGTCACT | SEQ ID | EE3179 | 0.57 | 0.53 | 0.60 |
| | | | NO: 573 | | | | |
| JERECRSTR | S000384 | CTCTTAGACCGCCTTCTTTGAAAG | SEQ ID | EE3271 | 2.02 | 1.89 | 2.15 |
| | | | NO: 9 | | | | |
| PE1ASPHYA3 | S000196 | GAAATAGCAAATGTTAAAAATA | SEQ ID | EE3391 | 0.68 | 0.64 | 0.72 |
| | | | NO: 5 | | | | |
| PE3ASPHYA3 | S000197 | CAGCTCCCATGGCTCTCCCATCCGCGCC | SEQ ID | EE3183 | 0.77 | 0.74 | 0.81 |
| | | GGT | NO: 574 | | | | |
| PIIATGAPB | S000382 | TTGGTTTTGATCAAAACCAA | SEQ ID | EE3170 | 0.27 | 0.23 | 0.31 |
| | | | NO: 575 | | | | |
| REGIONIOSOSEM | S000300 | CGGCGGCCTCGCCACG | SEQ ID | EE3157 | 0.85 | 0.82 | 0.88 |
| | | | NO: 576 | | | | |
| RSEPVGRP18 | S000289 | CATCCAACTTTCATATCCATGTGCTT | SEQ ID | EE3177 | 0.77 | 0.72 | 0.82 |
| | | | NO: 577 | | | | |
| RSRBNEXTA | S000243 | CAAACTCGTATATCCAT | SEQ ID | EE3163 | 0.66 | 10.62 | 0.70 |
| | | | NO: 578 | | | | |
| SBINPABC1 | S000435 | CACTAACACAAAGTAA | SEQ ID | EE3158 | 0.68 | 10.65 | 0.70 |
| | | | NO: 579 | | | | |
| SEIPVGRP18 | S000288 | ATAATGGGCCACACTGTGGGGCAT | SEQ ID | EE3394 | 0.93 | 0.87 | 0.99 |
| | | | NO: 10 | | | | |
| TEF1BOXATA1 | S000311 | ACAGGGGCATAATGGTAATTTAAA | SEQ ID | EE3176 | 1.46 | 1.40 | 1.52 |
| | | | NO: 580 | | | | |
| TEFBOXATEEF1AA1 | S000311 | AGGGGCATAATGGTAA | SEQ ID | EE3159 | 1.47 | 1.42 | 1.52 |
| | | | NO: 581 | | | | |
| WARBNEXTA | S000241 | GTACGTGTTATAAAACGTGT | SEQ ID | EE3171 | 0.81 | 0.78 | 0.84 |
| | | | NO: 582 | | | | |
| YREGIONNTPRB1B | S000365 | TGTGACATTGAAATTCTTTGACTTTA | SEQ ID | EE3178 | 1.02 | 0.98 | 1.06 |
| | | | NO: 583 | | | | |

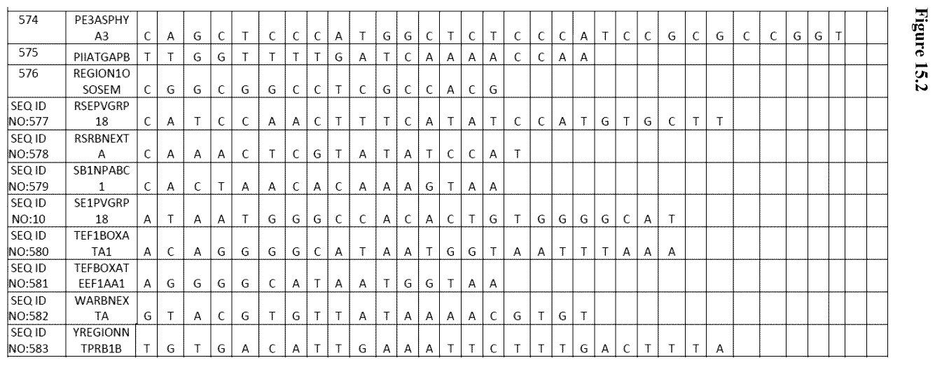

The results of the expression profiling indicated that the SEQ ID NO:551-SEQ ID NO:583 exhibited activity as a modulating element within a chimeric regulatory element (for example a cis-acting regulatory element and a promoter regulatory element) (FIG. 14). The cis-acting regulatory element drove robust expression of the fluorescent coding sequence in the maize protoplasts. Those with skill in the art would appreciate that these cis-acting regulatory elements would also drive robust expression of a coding sequence in other plants. As such, SEQ ID NO:551-583, SEQ ID NO:2-5 and SEQ ID NO:8-10 were exemplified for the first time as cis-acting regulatory elements capable of functioning as a modulating element within a chimeric regulatory element (for example a promoter and a cis-acting regulatory element) to drive expression of a coding sequence.

### Example 13: Identification of Cis-Acting Regulatory Elements That Increase Expression of a Coding Sequence Within Maize Protoplasts

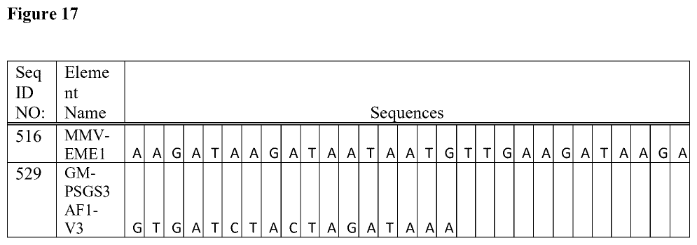

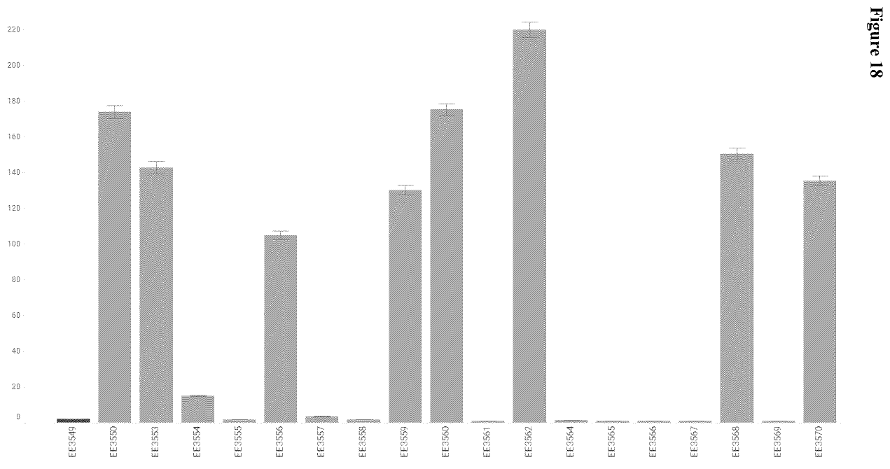

The GM-PSGS3AF1-V3 (SEQ ID NO:529) and the MMV-EME1 (SEQ ID NO:516) regulatory elements from Example 9 are cis-acting regulatory elements that were further screened in maize protoplasts and are listed in Table 9. An alignment of the sequences is provided in FIG. 17. Each regulatory element was engineered upstream of a regulatory element (for example a plant promoter) and used to produce standardized monocot protoplast testing vectors as described in Example 2 and 3, where the monocot promoter (Zea mays GOS2 promoter) was incorporated into construct design to drive expression in Zea mays protoplasts. The cis-acting regulatory elements were incorporated in duplicate within the construct. Those with skill in the art would appreciate that the cis-acting regulatory element could be include as a monomer, dimer, trimer, or in any copy number to drive robust expression of a coding sequence. Finally, the cis-acting regulatory elements were tested in a transient gene expression Zea mays leaf mesophyll protoplast platform as described in Examples 1 and 3 of patent application WO2018183878A1. The analysis was completed to test the vectors to determine whether the cis-acting regulatory elements could enhance the expression of a downstream coding sequence operably linked to the chimeric cis-acting regulatory element and another regulatory element (for example a promoter regulatory element) to function as a chimeric regulatory element.

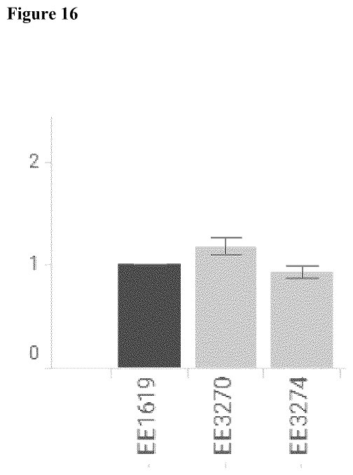

TABLE 9
Elements selected for testing.

| | | | | | | Fold | Fold |
|---|---|---|---|---|---|---|---|
| | | | | | | 95% | 95% |
| Element | Element | | SEQ ID | FIG. 16 | | Lower | Upper |
| Name | Origin | Sequence | NO: | Label: | Fold | CI | CI |
| No Element | N/A | N/A | N/A | EE1619 | 1 | 0 | 0 |
| MMV-EME1 | MMV FLt PRO | AAGATAAGATAATAATGTTGAAGATAAGA | 516 | EE3270 | 1.2 | 1.1 | 1.3 |
| GM- | Glycine max | GTGATCTACTAGATAAA | 529 | EE3274 | 0.9 | 0.9 | 1.0 |
| PSGS3AF1-V3 | | | | | | | |

The results of the expression profiling indicated that the SEQ ID NO:516 and SEQ ID NO:529 cis-acting regulatory elements exhibited activity as a modulating element within a chimeric regulatory element (for example a cis-acting regulatory element and a promoter regulatory element) (FIG. 16). The cis-acting regulatory element drove robust expression of the fluorescent coding sequence in the maize protoplasts. Those with skill in the art would appreciate that these cis-acting regulatory elements would also drive robust expression of a coding sequence in other plants. As such, SEQ ID NO:516 and SEQ ID NO:529 were exemplified for the first time as cis-acting regulatory elements capable of functioning as a modulating element within a chimeric regulatory element (for example a promoter and a cis-acting regulatory element) to drive expression of a coding sequence.

### Example 14: Identification of Cis-Acting Regulatory Elements That Increase Expression of a Coding Sequence Within Maize Protoplasts

Cis-acting regulatory elements were obtained by screening the MMV as-1 genetic element (SEQ ID NO:25) was screened against plant genomic sequence databases (e.g., rice, soybean, canola, maize, and sorghum). The screening was completed via a FIMO algorithm from the MEME suite with a cap of the first 100 hits (redundant) (Charles E. Grant, Timothy L. Bailey, and William Stafford Noble, “FIMO: Scanning for occurrences of a given motif”, Bioinformatics, 27(7):1017-1018, 2011). This analysis of the plant genomic sequence database resulted in the identification of a large number or sequences that shared relatively high levels of sequence identity to the MMV as-1 genetic element. The pool of sequences was further culled to identify putative cis-acting regulatory elements for testing in planta. These sequences were sorted and selected based on predicted characteristics of active regulatory elements, which included, for example, the intact bZIP binding motifs (e.g., ‘ACGT’) and sequences that shared high levels of sequence identity to the parent element for testing.

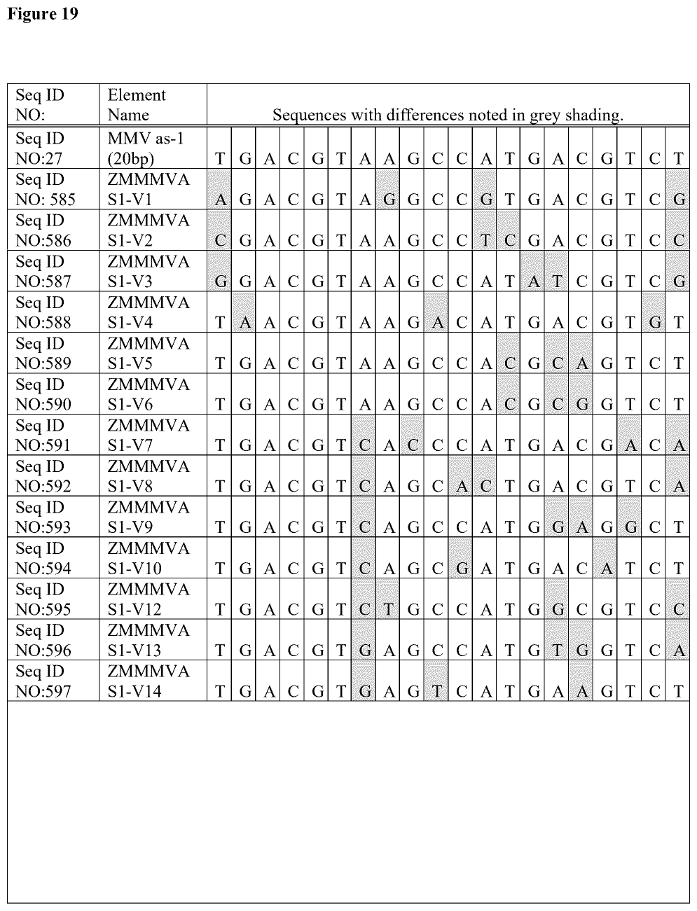

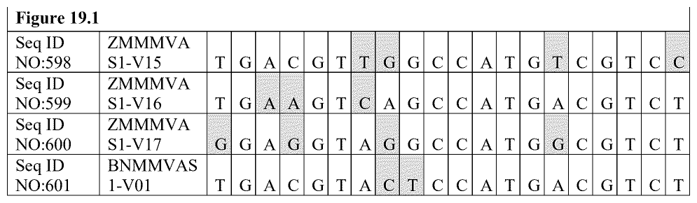

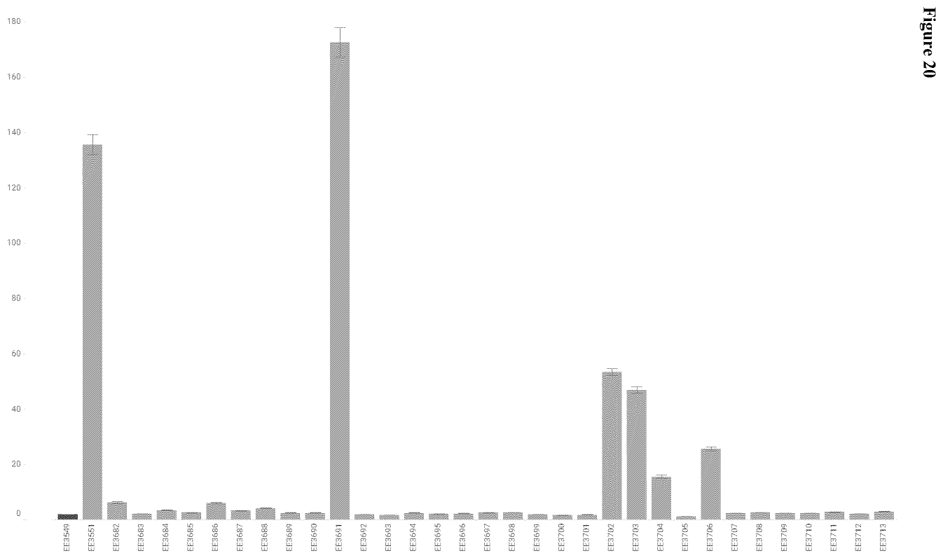

The cis-acting regulatory elements that were obtained are listed as SEQ ID NO:585-SEQ ID NO:601 and provided in Table 10. A sequence alignment of SEQ ID NO:585-SEQ ID NO:601 is provided as FIG. 19. Each cis-acting regulatory element was engineered upstream of a regulatory element (for example a plant promoter) and used to produce standardized monocot protoplast testing vectors as described in Example 2 and 3, where the monocot promoter (35S Minimal CAMV promoter of Yang, Y, Li, R and Qi, M. 2000. In vivo analysis of plant promoters and transcription factors by agroinfiltration of tobacco leaves. Plant J. 22(6):543-551) was incorporated into construct design to drive expression in Zea mays protoplasts. The cis-acting regulatory elements were incorporated in triplicate in the construct. Those with skill in the art would appreciate that the cis-acting regulatory element could be include as a monomer, dimer, trimer, or in any copy number to drive robust expression of a coding sequence. Finally, the cis-acting regulatory elements were tested in a transient gene expression Zea mays leaf mesophyll protoplast platform as described in Examples 1 and 3 of patent application WO2018183878A1. The analysis was completed to test the vectors to determine whether the cis-acting regulatory elements could enhance the expression of a downstream coding sequence operably linked to the chimeric cis-acting regulatory element and another regulatory element (for example a promoter regulatory element) to function as a chimeric regulatory element.

TABLE 10
Elements selected for testing.

| | | | | | | RFU | RFU |
|---|---|---|---|---|---|---|---|
| | | | | | | 95% | 95% |
| Element | Element | | SEQ | FIG. 18 | RFU | Lower | Upper |
| Name | Origin | Sequence | ID NO: | Label: | Mean | CI | CI |
| CaMV-as1 NI | Yang et al. | TGCTGTAAGGGATCTCGCAC | 584 | EE3549 | 2.1 | 2.1 | 2.1 |
| | 2000 | | | | | | |
| MMV as-1 | MMV FLt PRO | TGACGTAAGCCATGACGTCT | 27 | EE3550 | 173.8 | 170.2 | 177.6 |
| (20 bp) | | | | | | | |
| ZM-MMVAS1- | Zea mays | AGACGTAGGCCGTGACGTCG | 585 | EE3553 | 142.7 | 139.2 | 146.3 |
| V1 | | | | | | | |
| ZM-MMVAS1- | Zea mays | CGACGTAAGCCTCGACGTCC | 586 | EE3554 | 15.0 | 14.6 | 15.3 |
| V2 | | | | | | | |
| ZM-MMVAS1- | Zea mays | GGACGTAAGCCATATCGTCG | 587 | EE3555 | 1.9 | 1.8 | 1.9 |
| V3 | | | | | | | |
| ZM-MMVAS1- | Zea mays | TAACGTAAGACATGACGTGT | 588 | EE3556 | 104.9 | 102.5 | 107.3 |
| V4 | | | | | | | |
| ZM-MMVAS1- | Zea mays | TGACGTAAGCCACGCAGTCT | 589 | EE3557 | 3.7 | 3.6 | 3.8 |
| V5 | | | | | | | |
| ZM-MMVAS1- | Zea mays | TGACGTAAGCCACGCGGTCT | 590 | EE3558 | 1.8 | 1.7 | 1.8 |
| V6 | | | | | | | |
| ZM-MMVAS1- | Zea mays | TGACGTCACCCATGACGACA | 591 | EE3559 | 130.2 | 127.5 | 133.0 |
| V7 | | | | | | | |
| ZM-MMVAS1- | Zea mays | TGACGTCAGCACTGACGTCA | 592 | EE3560 | 175.2 | 171.8 | 178.7 |
| V8 | | | | | | | |
| ZM-MMVAS1- | Zea mays | TGACGTCAGCCATGGAGGCT | 593 | EE3561 | 1.1 | 1.0 | 1.1 |
| V9 | | | | | | | |
| ZM-MMVAS1- | Zea mays | TGACGTCAGCGATGACATCT | 594 | EE3562 | 219.9 | 215.8 | 224.1 |
| V10 | | | | | | | |
| ZM-MMVAS1- | Zea mays | TGACGTCTGCCATGGCGTCC | 595 | EE3564 | 1.4 | 1.4 | 1.5 |
| V12 | | | | | | | |
| ZM-MMVAS1- | Zea mays | TGACGTGAGCCATGTGGTCA | 596 | EE3565 | 1.1 | 1.1 | 1.1 |
| V13 | | | | | | | |
| ZM-MMVAS1- | Zea mays | TGACGTGAGTCATGAAGTCT | 597 | EE3566 | 1.2 | 1.2 | 1.2 |
| V14 | | | | | | | |
| ZM-MMVAS1- | Zea mays | TGACGTTGGCCATGTCGTCC | 598 | EE3567 | 1.0 | 1.0 | 1.0 |
| V15 | | | | | | | |
| ZM-MMVAS1- | Zea mays | TGAAGTCAGCCATGACGTCT | 599 | EE3568 | 150.3 | 147.1 | 153.6 |
| V16 | | | | | | | |
| ZM-MMVAS1- | Zea mays | GGAGGTAGGCCATGGCGTCT | 600 | EE3569 | 1.1 | 1.1 | 1.1 |
| V17 | | | | | | | |
| BN-MMVAS1- | Brassica napus | TGACGTACTCCATGACGTCT | 601 | EE3570 | 135.3 | 132.5 | 138.2 |
| V01 | | | | | | | |

The results of the expression profiling indicated that the MMV as-1 cis-acting regulatory element (SEQ ID NO:27) and the variants obtained from maize (SEQ ID NO:585-SEQ ID NO:601), exhibited activity as a modulating element within a chimeric regulatory element (for example a cis-acting regulatory element and a promoter regulatory element) (FIG. 18). This cis-acting regulatory element drove robust expression of the fluorescent coding sequence in maize protoplasts. Although there was some level of variation in the expression of the fluorescent coding sequence as compared to SEQ ID NO:27, many of the variant sequences expressed higher than the CaMV 35S minimal promoter control. As such, the MMV as-1 cis-acting regulatory element (SEQ ID NO:27) and MMV as-1 plant-derived variant cis-acting regulatory element (SEQ ID NO:585-601) were exemplified for the first time as cis-acting regulatory elements capable of functioning as a modulating element within a chimeric regulatory element (for example a promoter and a cis-acting regulatory element) to drive expression of a coding sequence.

### Example 15: Identification of Cis-Acting Regulatory Elements That Increase Expression of a Coding Sequence Within Maize Protoplasts

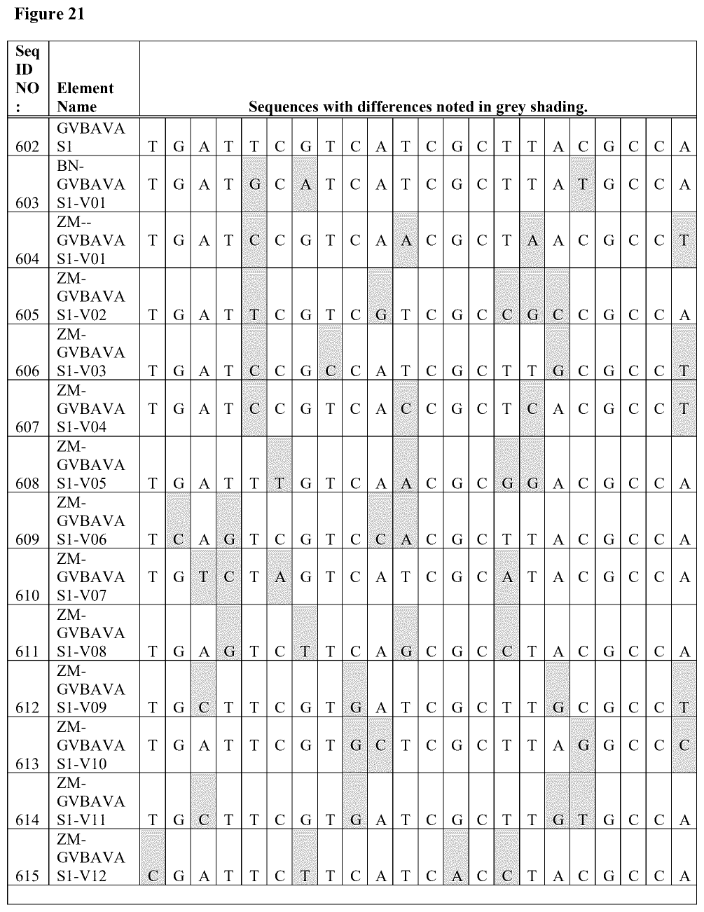

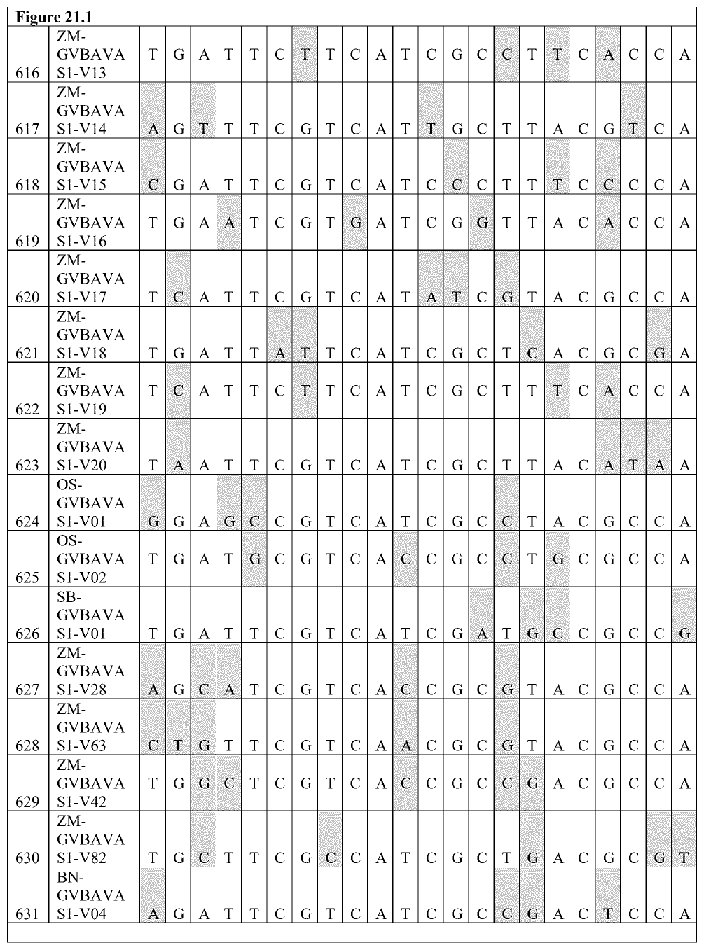

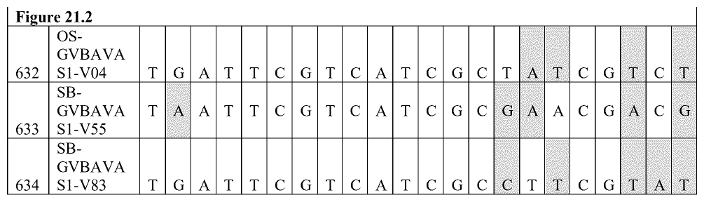

Cis-acting regulatory elements were obtained by screening the plant genomic sequence databases (e.g., rice, soybean, canola, maize, and sorghum). The cis-acting regulatory elements that were obtained are listed as SEQ ID NO:602-SEQ ID NO:634 and provided in Table 11. A sequence alignment of SEQ ID NO:602-SEQ ID NO:634 is provided as FIG. 21. Each cis-acting regulatory element was engineered upstream of a regulatory element (for example a plant promoter) and used to produce standardized monocot protoplast testing vectors as described in Example 2 and 3, where the monocot promoter (35S Minimal CAMV promoter) was incorporated into construct design to drive expression in Zea mays protoplasts. The cis-acting regulatory elements were incorporated in triplicate in the construct. Those with skill in the art would appreciate that the cis-acting regulatory element could be include as a monomer, dimer, trimer, or in any copy number to drive robust expression of a coding sequence. Finally, the cis-acting regulatory elements were tested in a transient gene expression Zea mays leaf mesophyll protoplast platform as described in Examples 1 and 3 of patent application WO2018183878A1. The analysis was completed to test the vectors to determine whether the cis-acting regulatory elements could enhance the expression of a downstream coding sequence operably linked to the chimeric cis-acting regulatory element and another regulatory element (for example a promoter regulatory element) to function as a chimeric regulatory element.

TABLE 11
Elements selected for testing.

| | | | | | | RFU 95% | RFU 95% |
|---|---|---|---|---|---|---|---|
| | | | SEQ ID | FIG. 20 | RFU | Lower | Upper |
| Element Name | Element Origin | Sequence | NO: | Label: | Mean | CI | CI |
| CaMV-as1_NI | Yang et al. | TGCTGTAAGGGATCTCGCAC | 584 | EE3549 | 1.9 | 1.9 | 1.9 |
| | 2000 | | | | | | |
| GVBAVAS1 | Gooseberry Vein | TGATTCGTCATCGCTTACGCCA | 602 | EE3551 | 135.5 | 3.6 | 3.7 |
| | Banding Virus | | | | | | |
| BN-GVBAVAS1- | Brassica napus | TGATGCATCATCGCTTATGCCA | 603 | EE3682 | 6.2 | 5.9 | 6.5 |
| V01 | | | | | | | |
| ZM-GVBAVAS1- | Zea mays | TGATCCGTCAACGCTAACGCCT | 604 | EE3683 | 2.2 | 2.2 | 2.3 |
| V01 | | | | | | | |
| ZM-GVBAVAS1- | Zea mays | TGATTCGTCGTCGCCGCCGCCA | 605 | EE3684 | 3.4 | 3.3 | 3.5 |
| V02 | | | | | | | |
| ZM-GVBAVAS1- | Zea mays | TGATCCGCCATCGCTTGCGCCT | 606 | EE3685 | 2.6 | 2.5 | 2.6 |
| V03 | | | | | | | |
| ZM-GVBAVAS1- | Zea mays | TGATCCGTCACCGCTCACGCCT | 507 | EE3686 | 6.1 | 5.9 | 6.3 |
| V04 | | | | | | | |
| ZM-GVBAVAS1- | Zea mays | TGATTTGTCAACGCGGACGCCA | 608 | EE3687 | 3.4 | 3.2 | 3.5 |
| V05 | | | | | | | |
| ZM-GVBAVAS1- | Zea mays | TCAGTCGTCCACGCTTACGCCA | 609 | EE3688 | 4.2 | 4.1 | 4.3 |
| V06 | | | | | | | |
| ZM-GVBAVAS1- | Zea mays | TGTCTAGTCATCGCATACGCCA | 610 | EE3689 | 2.5 | 2.5 | 2.6 |
| V07 | | | | | | | |
| ZM-GVBAVAS1- | Zea mays | TGAGTCTTCAGCGCCTACGCCA | 611 | EE3690 | 2.5 | 2.4 | 2.6 |
| V08 | | | | | | | |
| ZM-GVBAVAS1- | Zea mays | TGCTTCGTGATCGCTTGCGCCT | 612 | EE3691 | 172.6 | 167.4 | 177.9 |
| V09 | | | | | | | |
| ZM-GVBAVAS1- | Zea mays | TGATTCGTGCTCGCTTAGGCCC | 613 | EE3692 | 1.9 | 1.8 | 1.9 |
| V10 | | | | | | | |
| ZM-GVBAVAS1- | Zea mays | TGCTTCGTGATCGCTTGTGCCA | 614 | EE3693 | 1.7 | 1.7 | 1.8 |
| V11 | | | | | | | |
| ZM-GVBAVAS1- | Zea mays | CGATTCTTCATCACCTACGCCA | 615 | EE3694 | 2.5 | 2.4 | 2.6 |
| V12 | | | | | | | |
| ZM-GVBAVAS1- | Zea mays | TGATTCTTCATCGCCTTCACCA | 616 | EE3695 | 2.1 | 2.0 | 2.1 |
| V13 | | | | | | | |
| ZM-GVBAVAS1- | Zea mays | AGTTTCGTCATTGCTTACGTCA | 617 | EE3696 | 2.3 | 2.2 | 2.3 |
| V14 | | | | | | | |
| ZM-GVBAVAS1- | Zea mays | CGATTCGTCATCCCTTTCCCCA | 618 | EE3697 | 2.6 | 2.5 | 2.6 |
| V15 | | | | | | | |
| ZM-GVBAVAS1- | Zea mays | TGAATCGTGATCGGTTACACCA | 619 | EE3698 | 2.6 | 2.6 | 2.7 |
| V16 | | | | | | | |
| ZM-GVBAVAS1- | Zea mays | TCATTCGTCATATCGTACGCCA | 620 | EE3699 | 1.9 | 1.8 | 1.9 |
| V17 | | | | | | | |
| ZM-GVBAVAS1- | Zea mays | TGATTATTCATCGCTCACGCGA | 621 | EE3700 | 1.6 | 1.5 | 1.6 |
| V18 | | | | | | | |
| ZM-GVBAVASI- | Zea mays | TCATTCTTCATCGCTTTCACCA | 622 | EE3701 | 1.8 | 1.7 | 1.9 |
| V19 | | | | | | | |
| ZM-GVBAVAS1- | Zea mays | TAATTCGTCATCGCTTACATAA | 623 | EE3702 | 53.4 | 52.2 | 54.7 |
| V20 | | | | | | | |
| OS-GVBAVAS1- | Oryza sativa | GGAGCCGTCATCGCCTACGCCA | 624 | EE3703 | 46.9 | 45.6 | 48.2 |
| V01 | | | | | | | |
| OS-GVBAVAS1- | Oryza sativa | TGATGCGTCACCGCCTGCGCCA | 625 | EE3704 | 15.6 | 15.0 | 16.1 |
| V02 | | | | | | | |
| SB-GVBAVAS1- | Sorghum bicolor | TGATTCGTCATCGATGCCGCCG | 626 | EE3705 | 1.3 | 1.3 | 1.3 |
| V01 | | | | | | | |
| ZM-GVBAVAS1- | Zea mays | AGCATCGTCACCGCGTACGCCA | 627 | EE3706 | 25.7 | 25.0 | 26.4 |
| V28 | | | | | | | |
| ZM-GVBAVAS1- | Zea mays | CTGTTCGTCAACGCGTACGCCA | 628 | EE3707 | 2.5 | 2.4 | 2.5 |
| V63 | | | | | | | |
| ZM-GVBAVAS1- | Zea mays | TGGCTCGTCACCGCCGACGCCA | 629 | EE3708 | 2.6 | 2.6 | 2.7 |
| V42 | | | | | | | |
| ZM-GVBAVAS1- | Zea mays | TGCTTCGCCATCGCTGACGCGT | 630 | EE3709 | 2.4 | 2.4 | 2.5 |
| V82 | | | | | | | |
| BN-GVBAVAS1- | Brassica napus | AGATTCGTCATCGCCGACTCCA | 631 | EE3710 | 2.4 | 2.4 | 2.5 |
| V04 | | | | | | | |
| OS-GVBAVAS1- | Oryza sativa | TGATTCGTCATCGCTATCGTCT | 632 | EE3711 | 2.8 | 2.7 | 2.9 |
| V04 | | | | | | | |
| SB-GVBAVAS1- | Sorghum bicolor | TAATTCGTCATCGCGAACGACG | 633 | EE3712 | 2.2 | 2.1 | 2.2 |
| V55 | | | | | | | |
| SB-GVBAVAS1- | Sorghum bicolor | TGATTCGTCATCGCCTTCGTAT | 634 | EE3713 | 3.0 | 2.9 | 3.1 |
| V83 | | | | | | | |

The results of the expression profiling indicated that the cis-acting regulatory elements (SEQ ID NO:602-SEQ ID NO:634), exhibited activity as a modulating element within a chimeric regulatory element (for example a cis-acting regulatory element and a promoter regulatory element) (FIG. 20). Although there was some level of variation in the expression of the fluorescent coding sequence as compared to SEQ ID NO:584, most of the variant sequences expressed higher than the CaMV 35S minimal promoter control. The cis-acting regulatory elements were shown to drive robust expression of the fluorescent coding sequence in maize protoplasts. Those with skill in the art would appreciate that these cis-acting regulatory elements would also drive robust expression of a coding sequence in other plants. As such, the plant-derived variant cis-acting regulatory element (SEQ ID NO:602-SEQ ID NO:634) were exemplified for the first time as cis-acting regulatory elements capable of functioning as a modulating element within a chimeric regulatory element (for example a promoter and a cis-acting regulatory element) to drive expression of a coding sequence.

### Example 16: Identification of Additional Cis-Acting Regulatory Elements That Increase Expression of a Coding Sequence

Additional cis-acting regulatory elements are obtained from the PLACE database. The cis-acting regulatory elements that are obtained are listed as SEQ ID NOs:39-507 or SEQ ID NOs:635-2268. These regulatory elements are engineered in combination with promoter and used to produce a chimeric regulatory element. Those with skill in the art would appreciate that the cis-acting regulatory element could be included as a monomer, dimer, trimer, or in any copy number to drive robust expression of a coding sequence. The chimeric regulatory elements are engineered into standardized dicot or monocot protoplast testing vectors as described in Example 2 and 3, or any of the other Examples. Finally, the chimeric regulatory elements containing the novel cis-acting regulatory elements are tested in a soy hypocotyl protoplast assay as described in Example 4 to test the vectors to determine whether the cis-acting regulatory elements could enhance the expression of a downstream coding sequence operably linked to the chimeric cis-acting regulatory element and promoter. Likewise the chimeric regulatory elements are tested in a transient gene expression Zea mays leaf mesophyll protoplast platform as described in Examples 1 and 3 of patent application WO2018183878A1 to determine whether the cis-acting regulatory elements could enhance the expression of a downstream coding sequence operably linked to the chimeric cis-acting regulatory element and promoter. Those with skill in the art would appreciate that the chimeric regulatory elements could be tested in other plant expression systems.

The results of the expression profiling indicate that cis-acting regulatory elements of the subject disclosure (SEQ ID NOs:39-507 or SEQ ID NOs:635-2268) exhibit enhanced activity as a modulating element within a chimeric regulatory element. These specific cis-acting regulatory elements drive robust expression of the fluorescent coding sequence. As such, these newly identified cis-acting regulatory elements are exemplified for the first time as cis-acting regulatory elements capable of functioning as a modulating element within a chimeric regulatory element (for example a promoter and a cis-acting regulatory element) to drive expression of a coding sequence.

### Example 17: Transgenic Plants Containing Cis-Acting Regulatory Element That Increase Expression of a Coding Sequence

Construction of plant transformation vectors. Gene expression cassettes containing an active cis-acting regulatory element fused to a promoter (e.g. maize ubiquitin 1 promoter, 35S Cauliflower Mosaic Virus; Sugarcane bacilliform badnavirus promoter; promoters from rice actin genes; ubiquitin gene promoters; pEMU promoters; MAS promoter; maize H3 histone promoter; ALS promoter; phaseolin gene promoter; CAB promoter; RUBISCO promoter; LAT52 promoter; Zm13 promoter; and/or APG promoter are non-limiting examples of promoters known in the art that may be selected to drive expression of the gene of interest) are assembled onto plasmid vectors using standard molecular cloning methods. A gene of interest is cloned in directly downstream of the cis-acting regulatory element fused to a promoter. Next, a 3′ untranslated region (e.g., a ZmPER5 3′UTR; AtUbi103′UTR; AtEf1 3′UTR; or StPinII 3′UTR are non-limiting examples of 3′UTRs known in the art that may be selected to terminate expression of the gene of interest) is used to terminate transcription of the gene of interest. The completion of the gene expression cassettes are performed using standard molecular cloning methods and may include additional gene expression cassettes linked in trans or in cis to other gene expression cassettes that express other agronomically important traits and/or selectable markers.

Plant transformation. Further provided are non-limiting examples of plant transformation protocols for transformation of the gene expression cassettes containing the cis-acting regulatory element fused to a promoter. In light of the subject disclosure, additional crops can be transformed according to embodiments of the subject disclosure using techniques that are known in the art. Plant transformation of monocot plant species can be achieved. For Agrobacterium-mediated transformation of rye, see, e. g., Popelka J C, Xu J, Altpeter F., “Generation of rye with low transgene copy number after biolistic gene transfer and production of (Secale cereale L.) plants instantly marker-free transgenic rye,” Transgenic Res. 2003 October; 12(5):587-96.). For Agrobacterium-mediated transformation of sorghum, see, e. g., Zhao et al., “Agrobacterium-mediated sorghum transformation,” Plant Mol Biol. 2000 December; 44(6):789-98. For Agrobacterium-mediated transformation of barley, see, e. g., Tingay et al., “Agrobacterium tumefaciens-mediated barley transformation,” The Plant Journal, (1997) 11: 1369-1376. For Agrobacterium-mediated transformation of wheat, see, e.g., Cheng et al., “Genetic Transformation of Wheat Mediated by Agrobacterium tumefaciens,” Plant Physiol. 1997 November; 115(3):971-980. For Agrobacterium-mediated transformation of tobacco, see, e. g., U.S. Pat. App. Pub. No. US 2013/0157369 A1. For Agrobacterium-mediated transformation of rice, see, e.g., Hiei et al., “Transformation of rice mediated by Agrobacterium tumefaciens,” Plant Mol. Biol. 1997 September; 35(1-2):205-18. Plant transformation of dicot plant species can be achieved. For Agrobacterium-mediated transformation of Arabidopsis, see, e. g., Clough, S. J., & Bent, A. F. (1998). Floral dip: a simplified method for Agrobacterium-mediated transformation of Arabidopsis thaliana. The plant journal, 16(6), 735-743. For Agrobacterium-mediated transformation of cotton, see, e. g., Tohidfar, M., Mohammadi, M., & Ghareyazie, B. (2005). Agrobacterium-mediated transformation of cotton (Gossypium hirsutum) using a heterologous bean chitinase gene. Plant cell, tissue and organ culture, 83(1), 83-96. For Agrobacterium-mediated transformation of soybean, see, e. g., U.S. Pat. App. Pub. No. US 2014/0173780 A1. For Agrobacterium-mediated transformation of tobacco, see, e. g., An, G. (1985). High efficiency transformation of cultured tobacco cells. Plant Physiology, 79(2), 568-570.

Corn may be transformed with gene expression cassettes containing the cis-acting regulatory element fused to a chimeric regulatory element (for example a promoter and a cis-acting regulatory element) by utilizing the same techniques previously described in Example #8 of patent application WO 2007/053482.

Soybean may be transformed with gene expression cassettes containing the cis-acting regulatory element fused to a chimeric regulatory element (for example a promoter and a cis-acting regulatory element) by utilizing the same techniques previously described in Example #11 or Example #13 of patent application WO 2007/053482.

Cotton may be transformed gene expression cassettes containing the cis-acting regulatory element fused to a chimeric regulatory element (for example a promoter and a cis-acting regulatory element) by utilizing the same techniques previously described in Examples #14 of U.S. Pat. No. 7,838,733 or Example #12 of patent application WO 2007/053482 (Wright et al.).

Canola may be transformed with gene expression cassettes containing the cis-acting regulatory element fused to a chimeric regulatory element (for example a promoter and a cis-acting regulatory element) by utilizing the same techniques previously described in Example #26 of U.S. Pat. No. 7,838,733 or Example #22 of patent application WO 2007/053482 (Wright et al.).

Wheat may be transformed with gene expression cassettes containing the cis-acting regulatory element fused to a chimeric regulatory element (for example a promoter and a cis-acting regulatory element) by utilizing the same techniques previously described in Example #23 of patent application WO 2013/116700A1 (Lira et al.).

Rice may be transformed with gene expression cassettes containing the cis-acting regulatory element fused to a chimeric regulatory element (for example a promoter and a cis-acting regulatory element) by utilizing the same techniques previously described in Example #19 of patent application WO 2013/116700A1 (Lira et al.).

Arabidopsis may be transformed with gene expression cassettes containing the cis-acting regulatory element fused to a chimeric regulatory element (for example a promoter and a cis-acting regulatory element) by utilizing the same techniques previously described in Example #7 of patent application WO 2013/116700A1 (Lira et al.).

Tobacco may be transformed with gene expression cassettes containing the cis-acting regulatory element fused to a chimeric regulatory element (for example a promoter and a cis-acting regulatory element) by utilizing the same techniques previously described in Example #10 of patent application WO 2013/116700A1 (Lira et al.).

The Latin names for these and other plants are given below. It should be clear that other (non-Agrobacterium) transformation techniques can be used to transform gene expression cassettes containing the cis-acting regulatory element fused to a chimeric regulatory element (for example a promoter and a cis-acting regulatory element), for example, into these and other plants. Examples include, but are not limited to; Maize (Zea mays), Wheat (Triticum spp.), Rice (Oryza spp. and Zizania spp.), Barley (Hordeum spp.), Cotton (Abroma augusta and Gossypium spp.), Soybean (Glycine max), Sugar and table beets (Beta spp.), Sugar cane (Saccharum officinarum), Tomato (Lycopersicon esculentum and other spp., Physalis ixocarpa, Solanum incanum and other spp., and (Lyphomandra betacea), Potato (Solanum tuberosum), Sweet potato (Ipomoea batatas), Rye (Secale spp.), Peppers (Capsicum annuum, chinense, and frutescens), Lettuce (Lactuca sativa, perennis, and pulchella), Cabbage (Brassica spp.), Celery (Apium graveolens), Eggplant (Solanum melongena), Peanut (Arachis hypogea), Sorghum (Sorghum spp.), Alfalfa (Medicago sativa), Carrot (Daucus carota), Beans (Phaseolus spp. and other genera), Oats (Avena sativa and strigosa), Peas (Pisum, Vigna, and Tetragonolobus spp.), Sunflower (Helianthus annuus), Squash (Cucurbita spp.), Cucumber (Cucumis sativa), Tobacco (Nicotiana spp.), Arabidopsis (Arabidopsis thaliana), Turfgrass (Lolium, Agrostis, Poa, Cynodon, and other genera), Clover (Trifolium), Vetch (Vicia). Transformation of such plants, with gene expression cassettes containing the cis-acting regulatory element fused to a promoter to produce a chimeric regulatory element, for example, is contemplated in embodiments of the subject disclosure.

Use of the gene expression cassettes containing the cis-acting regulatory element fused to a promoter to produce a chimeric regulatory element can be deployed in many deciduous and evergreen timber species. Such applications are also within the scope of embodiments of this disclosure. These species include, but are not limited to; alder (Alnus spp.), ash (Fraxinus spp.), aspen and poplar species (Populus spp.), beech (Fagus spp.), birch (Betula spp.), cherry (Prunus spp.), eucalyptus (Eucalyptus spp.), hickory (Carya spp.), maple (Acer spp.), oak (Quercus spp.), and pine (Pinus spp.).

Use of the gene expression cassettes containing the cis-acting regulatory element fused to a promoter can be deployed in ornamental and fruit-bearing species. Such applications are also within the scope of embodiments of this disclosure. Examples include, but are not limited to; rose (Rosa spp.), burning bush (Euonymus spp.), petunia (Petunia spp.), begonia (Begonia spp.), rhododendron (Rhododendron spp.), crabapple or apple (Malus spp.), pear (Pyrus spp.), peach (Prunus spp.), and marigolds (Tagetes spp.).

Molecular analyses. Molecular analysis of transformed plant tissues are performed on samples obtained from plants materials transformed with gene expression cassettes containing the cis-acting regulatory element fused to a promoter to confirm the presence and copy number of a stably integrated cis-acting regulatory element fused to a promoter and to quantitate the expressed quantity of the protein expressed from the gene of interest being produced in the plant cell. Various assays are known in the art and can be utilized for molecular analysis of the cis-acting regulatory element fused to a promoter within plant material. As such, the cis-acting regulatory element is exemplified for the first time as a cis-acting regulatory element capable of functioning as a modulating element within a chimeric regulatory element (for example a chimeric promoter) to drive expression of a coding sequence.

While the present disclosure may be susceptible to various modifications and alternative forms, specific embodiments have been described by way of example in detail herein. However, it should be understood that the present disclosure is not intended to be limited to the particular forms disclosed. Rather, the present disclosure is to cover all modifications, equivalents, and alternatives falling within the scope of the present disclosure as defined by the following appended claims and their legal equivalents.

1. A chimeric regulatory molecule wherein the molecule comprises a nucleic acid sequence comprising at least 95% identity to SEQ ID NO:9, SEQ ID NOs:25-583, SEQ ID NOs:585-2268.

2. The chimeric regulatory molecule of claim 1, comprising SEQ ID NO:9, SEQ ID NOs:25-583, SEQ ID NOs:585-2268.

3. The chimeric regulatory molecule of claim 1, consisting of SEQ ID NO:9, SEQ ID NOs:25-583, SEQ ID NOs:585-2268.

4. The chimeric regulatory molecule of claim 1, wherein the nucleic acid sequence comprising at least 95% identity to SEQ ID NO:9, SEQ ID NOs:25-583, SEQ ID NOs:585-2268 is an enhancer.

5. The chimeric regulatory molecule of claim 1, comprising a promoter operably linked to the nucleic acid sequence comprising at least 95% identity to SEQ ID NO:9, SEQ ID NOs:25-583, SEQ ID NOs:585-2268.

6. A gene expression cassette comprising the chimeric regulatory molecule of claim 1.

7. The gene expression cassette of claim 6, wherein the nucleic acid sequence comprising at least 95% identity to SEQ ID NO:9, SEQ ID NOs:25-583, SEQ ID NOs:585-2268 is operably linked to a promoter, intron, of 5′UTR.

8. The gene expression cassette of claim 6, wherein the chimeric regulatory molecule is operably linked to a heterologous coding sequence.

9. The gene expression cassette of claim 8, wherein the heterologous coding sequence is a gene of interest.

10. The gene expression cassette of claim 9, wherein the gene of interest encodes a selectable marker protein, an insecticidal resistance protein, a herbicide tolerance protein, a nitrogen use efficiency protein, a water use efficiency protein, a small RNA molecule, a nutritional quality protein, or a DNA binding protein.

11. The gene expression cassette of claim 6, wherein the gene expression cassette is engineered within a recombinant vector.

12. The gene expression cassette of claim 11, wherein the vector is selected from the group consisting of a plasmid, a cosmid, a bacterial artificial chromosome, a virus, and a bacteriophage.

13. A transgenic plant cell comprising the gene expression cassette of claim 6.

14. The transgenic plant cell of claim 13, wherein said plant cell is a monocotyledonous plant.

15. The transgenic plant cell of claim 13, wherein said plant cell is a dicotyledonous plant.

16. The transgenic plant of claim 13, wherein the gene expression cassette is expressed constitutively.

17. A transgenic plant stably transformed with the gene expression cassette of claim 6.

18. The transgenic plant of claim 17, wherein said plant is a monocotyledonous plant.

19. The transgenic plant of claim 17, wherein said plant is a dicotyledonous plant.

20. The transgenic plant of claim 17, wherein the gene expression cassette is expressed constitutively.
